(19) United States
(12) Patent Application Publication
(10) Pub. No.: US 2021/0108274 A1
(43) Pub. Date: Apr. 15, 2021

(54) PANCREATIC DUCTAL ADENOCARCINOMA EVALUATION USING CELL-FREE DNA HYDROXYMETHYLATION PROFILE

(71) Applicant: Bluestar Genomics, Inc., San Francisco, CA (US)

(72) Inventors: Patrick A. Arensdorf, San Francisco, CA (US); Samuel Levy, San Francisco, CA (US); Chin-Jen Ku, San Francisco, CA (US); Francois Collin, San Francisco, CA (US); Gulfem Guler, San Francisco, CA (US)

(73) Assignee: Bluestar Genomics, Inc., San Francisco, CA (US)

(21) Appl. No.: 17/131,287

(22) Filed: Dec. 22, 2020

Related U.S. Application Data
(63) Continuation-in-part of application No. 16/576,703, filed on Sep. 19, 2019.
(60) Provisional application No. 62/733,566, filed on Sep. 19, 2018.

(51) Int. Cl. C12Q 1/6886; G16B 5/20; G16B 25/10

(52) U.S. Cl. CPC C12Q 1/6886; C12Q 2600/154; G16B 25/10; G16B 5/20

(57) ABSTRACT

Disclosed herein are methods for identifying patients likely to have pancreatic ductal adenocarcinoma (PDAC) and patients likely to be at risk for developing PDAC. Related methods are also provided that pertain to patient monitoring, treatment evaluation, and an improved multi-cancer test., as are kits that are suitable for carrying out the aforementioned methods. The invention makes use of hydroxymethylation biomarkers, which in combination with one or more clinical parameters and optionally one or more additional types of biomarkers and/or patient-specific risk factors, exhibit a hydroxymethylation level that correlates with the presence of or risk of developing PDAC.

### CROSS-REFERENCE TO RELATED APPLICATION

This is a continuation-in-part of U.S. Ser. No. 16/576,703, filed Sep. 19, 2019, which claims priority under 35 U.S.C. § 119(e)(1) to provisional U.S. Patent Application Ser. No. 62/733,566, filed Sep. 19, 2018, the disclosures of which are incorporated by reference in their entireties.

### TECHNICAL FIELD

The present invention relates generally to pancreatic cancer, and more particularly relates to the diagnosis, assessment, and management of pancreatic ductal adenocarcinoma using a non-invasive, epigenetic analysis of circulating cell-free DNA.

### BACKGROUND

Translational research using genomic and proteomic technologies has provided novel molecular insights into the pathogenesis of pancreatic cancer and the biology of the local tumor microenvironment, but has yet to yield robust diagnostic biomarkers to impact early diagnosis of a disease. This is reflected by a very low overall 5-year survival rate of 8.5%; see “Cancer Stat Facts: Pancreas Cancer” (National Cancer Institute Surveillance, Epidemiology, and End Results Program, 2017), retrieved on Oct. 16, 2017 from seer.cancer.gov/statfacts/html/pancreas.html. Pancreatic cancer often presents late and has few symptoms, at which point only 10% to 20% of patients are eligible for surgical resection.

The pancreas consists of acinar cells, ductal cells, centro-acinar cells, endocrine islets, and stellate cells. The majority of pancreatic cancers are adenocarcinomas, with pancreatic ductal adenocarcinoma (hereinafter referred to as PDAC) and its variants accounting for more than 90% of all pancreatic malignancies (Tempero et al. (2017) Journal of the Comprehensive Cancer Network 15(8): 1028-1060), with the next most common pathology being neuroendocrine tumors, followed by colloid carcinomas, solid-pseudopapillary tumors, acinar cell carcinomas, and pancreatoblastomas (Kleef et al. (2016), Nature Reviews Disease Primers: Pancreatic Cancer 2: 1-22). Tobacco smoking confers a two- to three-fold higher risk of pancreatic cancer and also demonstrates a dose-risk relationship, while contributing to approximately 15 to 30% of cases (ibid.), with smokers diagnosed 8 to 15 years younger than non-smokers (Anderson et al. (2012) Am. J. Gastroenterol 107(11):1730-39; Maisonneuve et al. (2010) Dig Dis 28(405):645-56). A family history of pancreatitis is contributory in approximately 10% of cases, and germline mutations in genes such as BRCA2, BRCA1, CDKN2A, ATM, STK11, PRSS1, MLH1 and PALB2 are also associated with pancreatic cancer with variable penetrance (Kleef, supra).

Age is a significant risk factor for pancreatic cystic lesions (PCLs) and pancreatic cancer. Zerboni et al. (2016) Abstracts/Pancreatology 16:S104 (Abstract ID: 1665) did a meta-analysis of 10 studies showing an overall prevalence of PCLs of 11%, with a higher rate of 16% in studies investigating subjects with a mean age greater than 55 years old. Studies using modern imaging technologies such as Magnetic Resonance Imaging (MRI) with contrast medium and cholangiopancreatography (MRCP) reported a significantly higher pooled prevalence of PCLs at 26% of subjects. Other known risk factors include, without limitation, diabetes mellitus, chronic pancreatitis, and obesity.

The management of PDAC presents physicians with challenges along the entire clinical spectrum, including early detection in high risk individuals, early diagnosis of patients with symptoms or imaging findings, prognostication of outcomes, and prediction of therapeutic responsiveness, which collectively have engendered intense research efforts to identify and validate biomarkers with sufficient clinical performance metrics to improve decision algorithms. Current guidelines in PDAC management are primarily limited to two biomarker recommendations: carbohydrate antigen 19-9 (CA19-9 or sialyl Lewis antigen) and carcinoembryonic antigen (CEA). CA19-9 is relied upon to guide surgery decisions, use of adjuvant therapy, or the detection of post-operative tumor recurrence; however, its utility is limited because 10% of the population does not secrete the antigen. See Swords et al. (2016) Onco Targets Ther 9: 7459-67 and U.S. Pat. No. 8,632,98. Furthermore, the restricted sensitivity and specificity of CA 19-09 as a biomarker for pancreatic cancer suggests limited diagnostic potential. CEA levels are assessed in pancreatic cyst fluid and then combined with imaging and clinical parameters to distinguish mucinous and non-mucinous cysts in order to mitigate risk (Fonseca et al. (2018) Pancreas 47(3): 272-79; Elta et al. (2018) Am. J. Gastroenterology 113: 464-79). However, CEA level does not correlate with the extent of disease (Schlieman et al. (2003) Arch Surg. 138)9): 951-56). Furthermore, while both tumor markers, if elevated, are useful in following patients with known disease, neither CA19-9 nor CEA has the sensitivity and specificity needed for use in screening patients to detect pancreatic cancer.

Among the inherited risk factors for pancreatic cancer are genomic mutations such as BRCA2, which confers a 3.5-fold risk in carriers, with the probability of a germline mutation between 6 to 12% in PDAC patients with a first-degree relative diagnosed with PDAC (Lal et al. (2000) Cancer Res 60: 409-416). Molecular analyses of pancreatic cancer genomes reveal activating mutations in KRAS and inactivation of CDKN2A, TP53 and SMAD4, either through point mutation or copy number changes at >50% population frequency (Blankin et al. (2012) Nature 491(7424): 399-405; Waddell et al. (2015) Nature 518(7540):495-501; Jones et al. (2008) Science 321(5897):1801-06). However, mutational heterogeneity along with lack of disease specificity due to pleiotropy renders this subset of genes inefficient in diagnosing patients. Molecular subtyping of pancreatic tumors using mutational-based data (Waddell (2015), supra) or gene expression signatures (REF), has not yet seen clinical application.

There remains an unmet and pressing need in the art for improved methods of detecting, diagnosing, predicting, assessing, treating, and monitoring pancreatic cancer, particularly PDAC. An ideal method would be reliable and non-invasive, optimally enabling analysis of tumor, microenvironment, pancreas, and immune cell DNA to identify genetic and epigenetic information that correlates with PDAC or an aspect thereof.

### SUMMARY OF THE INVENTION

Tumor and normal cell DNA is released into the bloodstream, and a cell-free DNA (cfDNA) sample extracted therefrom can be analyzed with respect to genetic and epigenetic signatures. Epigenetic signatures include, by way of example, DNA methylation, i.e., the conversion of cytosine to 5-methylcytosine (5mC), and DNA hydroxymethylation, the oxidation of 5mC to 5-hydroxymethylcytosine (5hmC), mediated in the mammalian genome by the TET (Ten-Eleven Translocation) family of enzymes. Such signatures may come from cells that are normal, or from a tumor, the tumor microenvironment, the affected organ, or the immune system, all of which may change in response to health conditions such as in the case of pancreatic cancer.

The present invention is predicated on the discovery of a set of hydroxymethylation biomarkers that in combination with one or more clinical parameters and optionally one or more other types of biomarkers and/or patient-specific risk factors, exhibits a hydroxymethylation level that correlates in some way with pancreatic cancer, particularly PDAC or another exocrine pancreatic cancer.

In a first embodiment, the invention provides a method for determining the likelihood that a patient has or will develop pancreatic ductal adenocarcinoma (PDAC), where the method comprises: obtaining a cfDNA sample from the patient; sequencing the patient cfDNA in a manner that identifies 5-hydroxymethylcytosine (5hmC) residues in the cfDNA; mapping the identified 5hmC residues to each of a plurality of loci in a reference hydroxymethylation profile, wherein each locus serves as a hydroxymethylation biomarker comprising a gene feature selected from a 3′UTR, a TTS, an intron, and a promoter, the gene feature associated with a gene implicated in pancreatic development, a gene related to cancer development, or a gene established to exhibit hyper-hydroxymethylation in PDAC; determining differences between hydroxymethylation of the patient cfDNA and the reference hydroxymethylation profile at each locus; and using the extent and location of the differences, calculating a probability score representing the likelihood that the patient has PDAC or will develop PDAC. In one embodiment, the probability score is calculated using a logistic regression analysis of the differences in hydroxymethylation level at each of the hydroxymethylation biomarkers.

The method may further include calculating the probability score from the extent and location of the differences in combination with at least one individual parameter correlated with the patient's risk of developing PDAC. An additional parameter may be a clinical parameter, an additional type of biological marker (i.e., a biological marker not related to hydroxymethylation), or a combination thereof. Clinical parameters include lesion size; lesion location; presence or absence of pancreatic inflammation; jaundice; presence or absence of other symptoms; patient age; weight; gender; ethnicity; family history; genetic mutations; diabetes; physical activity; diet; pro-inflammatory cytokine levels; and smoking status of the patient.

The selected loci that serve as hydroxymethylation biomarkers herein comprise loci selected for their relevance to PDAC. By “relevance” is meant that a hydroxymethylation biomarker locus, alone or in combination with one or more other hydroxymethylation biomarker loci, tends to exhibit an increase or decrease in hydroxymethylation in a manner that correlates with the risk, presence, absence, type, size, stage, invasiveness, grade, location, diagnosis, prognosis, outcome, and/or or likelihood of treatment responsiveness of pancreatic cancer.

It should be noted that some of the individual hydroxymethylation biomarkers disclosed herein may not have significant individual significance in the evaluation of a pancreatic lesion, but when used in combination with other hydroxymethylation biomarkers disclosed herein and clinical parameters impacting on the evaluation and monitoring of a pancreatic lesion, optionally further combined with one or more other types of biomarkers and/or patient-specific risk factors, become significant in discriminating as a method of the invention requires, e.g., between a subject who has pancreatic cancer and a subject who does not have pancreatic cancer, or between a subject who is likely to develop pancreatic cancer and a subject who is not likely to develop pancreatic cancer, etc. The methods of the present invention provide an improvement over currently available methods of evaluating the risk that a subject has pancreatic cancer or is likely to develop pancreatic cancer, by using the biomarkers defined herein.

The reference hydroxymethylation profile is a data set representing the hydroxymethylation level of each of a plurality of hydroxymethylation biomarkers, where the data set is a composite of hydroxymethylation profiles of a plurality of individuals having at least one shared characteristic. The reference profile may be a “normal” profile, i.e., a composite of hydroxymethylation profiles for individuals who do not have PDAC and/or are at low risk of developing PDAC. The reference profile may also be a PDAC profile, i.e., a composite of hydroxymethylation profiles for individuals who have PDAC or a risk factor correlating with the presence or likelihood of developing PDAC, such as profiles of individuals over a certain age, individuals with an identified lesion greater than a certain minimum size, individuals with pancreatic inflammation, and the like. That is, the reference profile may be a focused reference profile that can enhance the accuracy of the method. Different types of focused reference hydroxymethylation profiles may be constructed from different population groups, and an appropriate reference profile can then be selected for the evaluation of a particular patient. For patients who have chronic inflammation of the pancreas, i.e., chronic pancreatitis, a narrowed, or focused, reference profile generated from a set of individuals with chronic pancreatitis would be selected. Another focused reference profile might be constructed from a set of individuals who are diabetic, or obese, or cigarette smokers, and used in the evaluation of patients who are diabetic, obese, or smokers, respectively. These focused reference profiles can also be used in combination, depending on the attributes of the patient undergoing evaluation.

In one aspect of the embodiment, the cell-free DNA sample is extracted from a blood sample obtained from the patient. In another aspect, the cell-free DNA sample is extracted from a sample of pancreatic cyst fluid obtained from the patient.

In additional embodiments, the invention provides a method for identifying:

(a) the risk that a pancreatic lesion observed with an imaging scan, i.e., an identified pancreatic lesion, is cancerous;

(b) the risk that an identified noncancerous pancreatic lesion will become cancerous;

(c) the likelihood that a particular therapy for treating a subject with pancreatic cancer will be effective;

(d) the risk that a subject without an identified pancreatic lesion will, at some point, develop a pancreatic lesion, as well as

(e) the risk of that lesion becoming cancerous.

Observing changes in the biomarker set over time can provide (or in some cases confirm) additional information such as:

(f) the effectiveness of a therapy a subject is undergoing in connection with an identified pancreatic lesion;

(g) an increase or decrease in the risk that an identified pancreatic lesion will develop into cancer;

(h) an increase or decrease in the likelihood that a subject without an observed pancreatic lesion will develop a pancreatic lesion, and

(i) the risk of that lesion becoming cancerous; and

(j) a change in an identified pancreatic lesion, including (j-1) a change in the size of a pancreatic lesion, (j-2) a change in the stage of a cancerous pancreatic lesion, (j-3) a change in the grade of a cancerous pancreatic lesion; (j-4) a change in the degree of invasiveness of a cancerous pancreatic lesion; and (j-5) the change from a local or regionalized invasive cancerous pancreatic lesion to a metastatic pancreatic cancer; as well as (j-6) the identification or confirmation of the pancreas as the primary tissue of origin in a cancer first identified through metastasis (i.e., initially a cancer of unknown origin).

It will thus be appreciated that the methods herein are useful to (1) reduce the likelihood of carrying out an unnecessary surgical intervention, i.e., surgical resection of a benign pancreatic lesion, and (b) monitor post-surgical changes such as the development of additional lesions or the effectiveness of a post-surgical therapy (e.g., radiation, chemotherapy, other pharmacotherapy, etc.).

Equally important is the ability to identify a likely cancerous lesion at an early stage. These features of the invention in turn enable significant advances in the field, including the treatment of pancreatic cancer before the cancer has advanced or metastasized as well as a reduction in unnecessary surgery, i.e., removal of benign lesions.

In a further embodiment, then, the invention provides a method for reducing the risk that a pancreatic lesion surgically removed from a patient is benign, comprising, prior to surgery,

(a) obtaining a cell-free (cf)DNA sample from the patient;

(b) sequencing the patient cfDNA in a manner that identifies 5-hydroxymethylcytosine (5hmC) residues in the cfDNA;

(c) mapping the identified 5hmC residues to each of a plurality of loci in a reference hydroxymethylation profile, wherein each locus serves as a hydroxymethylation biomarker comprising a gene feature selected from a 3′UTR, a TTS, an intron, and a promoter, the gene feature associated with a gene implicated in pancreatic development, a gene related to cancer development, or a gene established to exhibit hyper-hydroxymethylation in PDAC;

(d) determining differences in extent and location between hydroxymethylation of the patient cfDNA and the reference hydroxymethylation profile at each locus; and

(e) using the extent and location of the differences, calculating a probability score representing the likelihood that the pancreatic lesion is benign; and

(f) carrying out surgical resection of the pancreatic lesion only if the probability score is greater than a value corresponding to a low risk of cancer.

In another embodiment, the invention provides a kit for carrying out any of the methods described herein in the analysis of a cell-free DNA sample obtained from a patient, where the kit comprises: at least one reagent for the determination of hydroxymethylation level at each of a plurality of hydroxymethylation biomarker loci in a cell-free DNA sample; a solid support for capturing affinity-tagged 5hmC-containing cell-free DNA in the sample; and written instructions for the use of the at least one reagent and the solid support in carrying out any of the methods described and claimed herein.

In a further embodiment, the invention provides an improved method for conducting a multi-cancer test from a cell-free DNA sample taken from a patient, i.e., a method for assessing the patient's PDAC status as provided herein combined with a method for assessing the likelihood that a patient has at least one additional type of cancer. The method can additionally include a step of eliminating false negatives and/or false positives for the at least one additional type of cancer.

The at least one additional type of cancer can be any type of cancer, including, without limitation, bladder cancer; cancers of the blood and bone marrow; brain cancer; breast cancer; cervical cancer; colorectal cancer; esophageal cancer; liver cancer; lung cancer; ovarian cancer; prostate cancer; renal cancer; skin cancer; testicular cancer; thyroid cancer; and uterine cancer.

In one aspect of this embodiment, the at least one additional type of cancer is selected from breast cancer, colorectal cancer, lung cancer, and prostate cancer.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIG. 1 schematically depicts the study cohorts employed in Example 1 herein. Cohorts: PDAC, n=51, Non-cancer, n=41. Pooled non-cancer replicates were included across multiple 5hmC assay processing and sequencing batches.

FIG. 2 schematically depicts the sample processing workflow used in Example 1, including two alternating gender-divided flow cell constructs for detection of sample swaps.

FIG. 3 is a histogram showing the mean peak counts of 5hmC loci across distinct genomic regions, for the two cohorts of Example 1, PDAC and non-cancer (identified in the figures as “PDAC” and “NC,” respectively. It may be seen that non-coding features have a larger number of peaks.

FIG. 4 is a histogram providing the results of the enrichment analysis described in Example 1, with the Y-axis value equal to the mean of log₂ (cancer/non-cancer). The histogram shows that gene-based features, SINEs, and Alus are enriched in 5hmC in both cancer and non-cancer cohorts, whereas intergenic regions, LINEs, and L1s are depleted of 5hmC peaks.

FIG. 5 provides box plots depicting statistically significant changes of 5hmC peaks in pancreatic cancer samples relative to non-cancer samples as observed using the gene sets and procedures of Example 1, in the promoter, LINE elements, exons, 3′UTR, and translation termination sites; here, the Y-axis value equal to log₂ (cancer/non-cancer). Promoter and LINE elements were found to exhibit a depletion of 5-hydroxymethylcytosine (i.e., a decrease in hydroxymethylation) in the cancer (PDAC) samples relative to the non-cancer samples, while 5hmC enrichment was observed in exons, 3′UTR, and translation termination sites. In each box plot herein, the line within the box represents the median of the data, while the lower limit of the box represents the lower quartile and the upper limit of the box represents the upper quartile. Normally distributed data are portrayed as an aligned dot plot with error bars representing standard deviation from the mean. The calculated p-values are provided above each plot.

FIG. 6 provides box plots depicting statistically significant changes of 5hmC peaks in functional regions across pancreatic stages, as discussed in Example 1.

FIG. 7 provides box plots depicting 5hmC peak depletion in H3K4me3 and H3K27ac histone marks in the PDAC cohort (top panel) and ongoing H3K4me3 depletion observed in later stage disease (bottom panel), as described in Example 1.

FIG. 8A and FIG. 8B, together FIG. 8, show 5hmC occupancy in the PANC-1 cell line and normal pancreas histone map depicting variable occupancy in H3K4Me3 (FIG. 8A) with depletion at the center of the mark and complementary increase in 5hmC in H3K4Me1 (FIG. 8B), as determined in Example 1. The results support a preferential increase in gene transcription in the PDAC cohort. The Y-axis values are equal to the normalized density of 5hmC counts in 10 bp windows. The dotted red lines=PDAC patient, one per line; the dotted blue lines=non-cancer patients, one per line; the solid red line=the average density of normalized 5hmC counts across all PDAC patients; and the solid blue line=the average density of normalized 5hmC counts across all non-cancer patients.

FIG. 9 is an MA plot showing all differentially represented genes observed using the procedures of Example 1 (adjusted p-value<0.05, NC=non-cancer cohort). The black points (top) mark genes with increased 5hmC density in PDAC versus NC, while the gray points (bottom) mark genes with decreased 5hmC density in PDAC versus NC. The distinction is significant at the adjusted p-value<0.05, as explained in Example 1.

FIG. 10 is a histogram showing the results of gene set enrichment analysis (GSEA) using differentially 5hmC-enriched genes, as determined in Example 1. The black bars represent the ratio of all pathways exhibiting reduced hydroxymethylation levels, and the gray bars represent the ratio of all pathways exhibiting higher hydroxymethylation levels, in PDAC samples relative to non-cancer samples. GSEA reveals that greater than 20% of KEGG (Kyoto Encyclopedia of Genes and Genomes) pathways are both up-represented and down-represented in hydroxymethylation levels in PDAC versus non-cancer samples. Also, greater than 30% of immune pathways were found to be down-represented in PDAC versus non-cancer samples. In FIG. 10, “Hallmark” refers to the Hallmark gene sets in the MSigDB collections; “C2” refers to curated gene sets inclusive of the Biocarta, KEGG and Reactome databases; “C5.BP” refers to the “biological processes” subset of the Gene Ontology (GO) Consortium annotated gene sets; “C6” MSigDB oncogenic signature of cellular pathways that are often dis-regulated in cancer; and “C7” (also referred to as “immuneSigDB”) refers to the database of gene sets that represent cell types, states, and perturbations within the immune system.

FIG. 11 is a dot plot providing the results of Principal Component Analysis (PCA) carried out using log [counts per million] on 13,180 genes with a statistically significant (FDR=0.05) increase or decrease in 5hmC in PDAC versus non-cancer samples, as explained in Example 1; the dot plot exhibits visible partitioning of PDAC samples from non-cancer samples.

FIG. 12 is a PCA dot plot carried out using log [counts per million] on 320 genes, a subset of the 13,180 genes that exhibited a statistically significant (FDR=0.05) increase or decrease in 5hmC in PDAC versus non-cancer samples, where the data was filtered for increased PDAC representation as follows: (1) (log₂ [5hmC-PDAC/5hmC-non-cancer]≥0.58; and (2) log₂ [average representation]≥5. The dot plot again shows good partitioning of PDAC samples from non-cancer samples despite an order of magnitude smaller gene set than used in the generation of FIG. 11.

FIG. 13 is a heatmap depicting the hierarchical clustering results obtained using the 320 genes selected for the PCA of FIG. 12 (the genes represent rows in the heatmap), to show how labeled samples (columns in the heat map) can be partitioned using log(CPM) 5mC counts. The heatmap shows near-perfect partitioning of the data, which in this case was that used by Stanford University in Song et al. (2017) Cell Research 27:1231-42 (Song et al. (2017)) (sometimes referred to herein as “the Stanford data”).

FIG. 14 is also a heatmap, prepared as explained above for FIG. 13, but using the data of Li et al. (2017) Cell Research 27:1243-1257 (“Li et al. (2017”) (sometimes referred to herein as “the Chicago data”). In contrast to the almost perfect partitioning of the Stanford data, the Chicago data gave somewhat incomplete partitioning.

FIGS. 15A and 15B, together FIG. 15, provide the results of predictive modeling using two regularization models, elastic net and lasso. FIG. 15A represents the training performed with 75% of the data, and FIG. 15B represents the test performed on the remaining 25% of the data, as described in Example 1 herein.

FIG. 16 gives the probability scores derived from each sample in the training data set of Example 1 using the elastic net and lasso regularization methods. Probability scores near 1 are predicted cancer samples, while probability scores close to zero are non-cancer samples. The horizontal gray line across each plot identified the Q3 probability score of the non-cancer samples.

FIG. 17, which includes FIG. 17A and FIG. 17B, represents the validation of the predictive models used with the Li et al. (2017) (Chicago) and Song et al. (2017) (Stanford) PDAC and non-cancer data sets, as explained in Example 1.

FIG. 18 presents a comparison of t-scores of 5hmC density fold difference between PDAC and non-cancer cohorts as found in Song et al. (2017) and Li et al. (2017), each compared to the results of Example 1. All gene scores are represented in gray, elastic net model genes are in medium gray and lasso model genes are in black. The size of each gray dot and each black dot represents the relative contribution of that gene to the model.

FIG. 19 illustrates in graph form the hydroxymethylation levels (“5hmC occupancy”) at loci associated with histone biomarkers H3K4me3, H3K4me1, and H3K27ac (observed in Example 1), and the similarity to an existing histone map from PANC-1 cell lines (LeRoy et al. (2013) Epigenetics & Chromatin 6:20).

FIG. 20 provides hydroxymethylation biomarker data obtained using the methods documented in Example 1 herein. The table of FIG. 20 identifies the genes by name and chromosome location and includes normalized values obtained with glmnet, glmnet2, glmnetF, and glmnet2F regularization methods; glmnetF and glmnet2F coefficients; mean and standard deviation; mean and standard deviation for the cancer cohort (identified as Mean-C and SD-C, respectively); mean and standard deviation for the non-cancer cohort (Mean-NC and SD-NC, respectively); vote, computed as the sum of the glmnetF and glmnet2F normalized values for each gene; and the ratio of cancer-to-non-cancer (C/NC) means.

FIG. 21 provides a list of hydroxymethylation biomarkers suitable for use in conjunction with the present invention, by gene name, location, and glmnet value, identified using Study Group 2 in Example 2.

FIG. 22 is analogous to FIG. 20 but provides the biomarker data for the 41 genes of Table 4, infra, using Study Group 3 in Example 3.

FIG. 23 pertains to the differential enrichment of 5hmC in genomic features in PDAC cfDNA compared with non-cancer cfDNA samples as observed in Example 4, with 5hmC peak enrichment analysis (y-axis=log₂ enrichment) showing that gene-based features and SINEs are enriched in 5hmC peaks in both PDAC and non-cancer cfDNA cohorts. Intergenic and LINEs are depleted of 5hmC peaks.

FIG. 24 also pertains to the differential enrichment of 5hmC in genomic features in PDAC cfDNA compared with non-cancer cfDNA samples as observed in Example 4, with the number of 5hmC peaks detected per million reads shown in both non-cancer (left); and PDAC (right) cohorts.

FIG. 25 also pertains to the differential enrichment of 5hmC in genomic features in PDAC cfDNA compared with non-cancer cfDNA samples as observed in Example 4, with box plots depicting statistically significant changes of 5hmC peaks over promoters, 3′UTR, intron and transcription termination site (TTS) regions in PDAC cfDNA compared to non-cancer cfDNA. Each dot represents an individual cfDNA sample.

FIG. 26 pertains to the differential 5hmC enrichment over chromatin states identified in PDAC primary tissue in PDAC cfDNA compared with non-cancer samples as described in Example 4, and shows: chromatin states observed in two primary PDAC tumor tissues as determined by six histone markers, H3K4me1, H3K4me3, H3K9me3, H3K27ac, H3K27me3 and H3K36me3; and box plots depicting 5hmC occupancy in PDAC and non-cancer (NC) cfDNA cohorts over the chromatin states determined in PDAC primary tissue samples (statistical significance in PDAC versus non-cancer cfDNA enrichment is denoted by ** (p value<0.00001) and * (p value<0.0001).

FIG. 27 pertains to differential 5hmC occupancy analysis in PDAC cfDNA as compared to cfDNA from non-cancer patients, as described in Example 4, and provides an MA-plot showing all genes with differential 5hmC representation. The darker gray (top) and lighter gray (bottom) respectively denote increased or decreased 5hmC density in PDAC compared to non-cancer with adjusted p-value<0.01.

FIG. 28 also pertains to differential 5hmC occupancy analysis in PDAC cfDNA as compared to cfDNA from non-cancer patients, as described in Example 4, and is an IGV genome browser snapshot of the YAP1 locus showing the increased 5hmC signal intensity in PDAC samples compared to non-cancer controls.

FIG. 29 also pertains to differential 5hmC occupancy analysis in PDAC cfDNA as compared to cfDNA from non-cancer patients, as described in Example 4, and provides the GSEA of 794 genes with the most statistically significant differential 5hmC representation (adjusted p-value<0.01) and filtered for fold change in 5hmC representation (|(5hmC-PDAC/5hmC-non-cancer)|≥1.5) and minimum average expression (log₂ (average representation)≥3.5) in PDAC cfDNA as compared to non-cancer samples.

FIG. 30 also pertains to differential 5hmC occupancy analysis in PDAC cfDNA as compared to cfDNA from non-cancer patients, as described in Example 4, and includes FIGS. 30A, 30B, and 30C, wherein:

FIG. 30A is an MDS of pancreatic cancer (orange) and non-cancer (blue cfDNA samples using 11,855 genes with statistically significant (adjusted p-value<0.01) increase or decrease in 5hmC; and

FIG. 30B and FIG. 30C provide MDS of pancreatic cancer (orange) and non-cancer (blue) cfDNA samples from Example 4 and Song et al. (2017), respectively, using 794 genes with statistically significant differential 5hmC representation (adjusted p-value<0.01) and filtered for fold change in 5hmC representation (|(5hmC-PDAC/5hmC-non-cancer)|>=1.5) and minimum average expression (log₂ (average representation)>=3.5).

FIG. 31 pertains to the identification of a 5hmC signature that differentiates PDAC cfDNA from non-cancer samples as described in Example 4, and shows predictive modeling using a regularized regression model (elastic net) on the discovery dataset with 38 PDAC and 41 non-cancer cfDNA samples.

FIG. 32 also pertains to the identification of a 5hmC signature that differentiates PDAC cfDNA from non-cancer samples as described in Example 4, and shows the probability scores derived from each sample in the data set using the elastic net (probability scores approaching 1 are predicted cancer samples, whereas probability scores close to zero are likely non-cancer samples).

FIG. 33 also pertains to the identification of a 5hmC signature that differentiates PDAC cfDNA from non-cancer samples as described in Example 4, and provides box plots of 5hmC coverage, expressed in log CPM, over BAGE5, RUNXT1, SLFN14, and CD22 in PDAC and non-cancer cfDNA cohorts as an example of representative top model features.

FIG. 34 also pertains to the identification of a 5hmC signature that differentiates PDAC cfDNA from non-cancer samples as described in Example 4, and provides an ROC curve for independent validation cohorts of 23 PDAC cfDNA and 205 non-cancer cfDNA processed using the method of Example 4.

FIG. 35 also provides an ROC curve, for independent validation cohorts of 7 PDAC cfDNA and 10 non-cancer cfDNA from Song et al. (2017).

FIG. 36 also pertains to the identification of a 5hmC signature that differentiates PDAC cfDNA from non-cancer samples as described in Example 4, and gives the predicted probability scores for non-cancer (n=10) and Stage I (n=15), Stage II (n=16), Stage III (n=8) and Stage IV (n=11) of cancer; the same samples were also analyzed for CA19-9 levels. Samples within the clinically defined normal range (0-37 U/ml) are denoted in black, and samples that are above the range are denoted in gray.

FIG. 37 relates to tissue-derived 5hmC features able to classify PDAC cfDNA, as described in Example 4, and includes FIGS. 37A, 37B, 37C, and 37D, as follows:

FIG. 37A shows the average FPKM values over the top 50 genes, with the highest 5hmC occupancy, compared with the 50 genes with the lowest 5hmC occupancy, in PDAC tumor tissues;

FIG. 37B are PCA plots showing non-cancer PDAC cfDNA cohorts using the top 50 (upper panel) and bottom 50 (bottom panel) genes as features; and

FIG. 37C provides ROC curves for a predictive model using features discovered in cfDNA, the top 50 genes with the highest 5hmC representation in tissue, and the 50 genes with the lowest 5hmC representation in tissue; and.

FIG. 37D also relates to tissue-derived 5hmC features able to classify PDAC cfDNA, as explained in Example 4, and provides a heatmap showing FPKM values of all the gene features (n=37) used in the cfDNA prediction model (represented in each row) in non-cancer cfDNA, PDAC cfDNA and PDAC tissue 5hmC profiles (represented in each column).

FIG. 38 is a heatmap showing FPKM values of all of the gene features (n=37) used in the cfDNA prediction model (represented in each row) in non-cancer cfDNA, PDAC cfDNA, and PDAC tissue 5hmC profiles (represented in each column).

FIG. 39 pertains to the differential enrichment of 5hmC in genomic features in PDAC cfDNA compared with non-cancer cfDNA samples, and illustrates the 5hmC peak distribution over genomic features determined as explained in Example 4.

FIG. 40 provides 5hmC log₂ enrichment data over functional genomic regions in PDAC and non-cancer cfDNA cohorts calculated over genomic background; box plots depict statistically significant changes of 5hmC peaks in functional genomic regions across pancreatic cancer stages (see Example 4).

FIG. 41 provides 5hmC log₂ enrichment across samples from different pancreatic cancer stages and non-cancer cfDNA over H3K4me3 and H3K4me1 regions as determined in PDAC primary tumor tissues A0440 and A0424.

FIG. 42 pertains to the partitioning of PDAC and non-cancer cfDNA samples using a small set of hydroxymethylated genes, and illustrates the hierarchical clustering of pancreatic cancer (magenta) and healthy (gray) cfDNA samples from the discovery cohort, employing 5hmC counts over 794 differentially hydroxymethylated genes, as described in Example 4.

FIG. 43 is a bar graph illustrating the distribution of patients over pancreatic cancer stages I-IV from the study of Example 4 (gray bars) and for the Stanford data (black bars), expressed as a percentage of total cohort in each study.

FIG. 44 is a heatmap showing hierarchical clustering of pancreatic cancer (magenta) and healthy (gray) cfDNA samples from Song et al. (2017) employing 5hmC counts over 794 differentially hydroxymethylated genes identified in Example 4.

### DETAILED DESCRIPTION OF THE INVENTION

1. Terminology and Overview:

Unless defined otherwise, all technical and scientific terms used herein have the meaning commonly understood by one of ordinary skill in the art to which the invention pertains. Specific terminology of particular importance to the description of the present invention is defined below. Other relevant terminology is defined in International Patent Publication No. WO 2017/176630 to Quake et al. for “Noninvasive Diagnostics by Sequencing 5-Hydroxymethylated Cell-Free DNA.” The aforementioned patent publication as well as all other patent documents and publications referred to herein are expressly incorporated by reference.

In this specification and the appended claims, the singular forms “a,” “an” and “the” include plural referents unless the context clearly dictates otherwise. Thus, for example, “an adapter” refers not only to a single adapter but also to two or more adapters that may be the same or different, “a template molecule” refers to a single template molecule as well as a plurality of template molecules, and the like.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

The term “sample” as used herein relates to a material or mixture of materials, typically, although not necessarily, in liquid form, containing one or more analytes of interest.

The term “biological sample” as used herein relates to a sample derived from a biological fluid, cell, tissue, or organ of a human subject, comprising a mixture of biomolecules including proteins, peptides, lipids, nucleic acids, and the like. Generally, although not necessarily, the sample is a blood sample such as a whole blood sample, a serum sample, or a plasma sample, or a sample of pancreatic cyst fluid.

A “nucleic acid sample” as that term is used herein refers to a biological sample comprising nucleic acids. The nucleic acid sample may be a cell-free nucleic acid sample that comprises nucleosomes, in which case the nucleic acid sample is sometimes referred to herein as a “nucleosome sample.” The nucleic acid sample may also be comprised of cell-free DNA wherein the sample is substantially free of histones and other proteins, such as will be the case following cell-free DNA purification. The nucleic acid samples herein may also contain cell-free RNA.

A “sample fraction” refers to a subset of an original biological sample, and may be a compositionally identical portion of the biological sample, as when a blood sample is divided into identical fractions. Alternatively, the sample fraction may be compositionally different, as will be the case when, for example, certain components of the biological sample are removed, with extraction of cell-free nucleic acids being one such example.

As used herein, the term “cell-free nucleic acid” encompasses both cell-free DNA and cell-free RNA, where the cell-free DNA and cell-free RNA may be in a cell-free fraction of a biological sample comprising a body fluid. The body fluid may be blood, including whole blood, serum, or plasma, or it may be urine, cyst fluid, or another body fluid. In many instances, the biological sample is a blood sample, and a cell-free nucleic acid sample, e.g., a cell-free DNA sample, is extracted therefrom using now-conventional means known to those of ordinary skill in the art and/or described in the pertinent texts and literature; kits for carrying out cell-free nucleic acid extraction are commercially available (e.g., the AllPrep® DNA/RNA Mini Kit and QIAmp DNA Blood Mini Kit, both available from Qiagen, or the MagMAX Cell-Free Total Nucleic Acid Kit and the MagMAX DNA Isolation Kit, available from ThermoFisher Scientific). Also see, e.g., Hui et al. Fong et al. (2009) Clin. Chem. 55(3):587-598. The cell-free DNA samples herein may also be obtained from other sources, e.g., pancreatic cyst fluid.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. Of particular interest herein are modified cytosine residues, including 5-methylcytosine and oxidized forms thereof, such as 5-hydroxymethylcytosine, 5-formylcytosine, and 5-carboxymethylcytosine.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, and up to about 10,000 or more bases composed of nucleotides, e.g., deoxyribonucleotides or ribonucleotide. Nucleic acids may be produced enzymatically, chemically synthesized, or naturally obtained.

The term “oligonucleotide” as used herein denotes a single-stranded multimer of nucleotide of from about 2 to 200 nucleotides, up to 500 nucleotides in length.

Oligonucleotides may be synthetic or may be made enzymatically, and, in some embodiments, are 30 to 150 nucleotides in length. Oligonucleotides may contain ribonucleotide monomers (i.e., may be oligoribonucleotides) and/or deoxyribonucleotide monomers. An oligonucleotide may be 10 to 20, 21 to 30, 31 to 40, 41 to 50, 51to 60, 61 to 70, 71 to 80, 80 to 100, 100 to 150 or 150 to 200 nucleotides in length, for example.

The term “hybridization” refers to the process by which a strand of nucleic acid joins with a complementary strand through base pairing as known in the art. A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.).

The terms “duplex” and “duplexed” are used interchangeably herein to describe two complementary polynucleotides that are base-paired, i.e., hybridized together. A DNA duplex is referred to herein as “double-stranded DNA” or “dsDNA” and may be an intact molecule or a molecular segment. For example, the dsDNA herein referred to as barcoded and adapter-ligated is an intact molecule, while the dsDNA formed between the nucleic acid tails of proximity probes in a proximity extension assay is a dsDNA segment.

The term “strand” as used herein refers to a single strand of a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, “positive” and “negative” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

“Adapters” as that term is used herein are short synthetic oligonucleotides that serve a specific purpose in a biological analysis. Adapters can be single-stranded or double-stranded, although the preferred adapters herein are double-stranded. In one embodiment, an adapter may be a hairpin adapter (i.e., one molecule that base pairs with itself to form a structure that has a double-stranded stem and a loop, where the 3′ and 5′ ends of the molecule ligate to the 5′ and 3′ ends of a double-stranded DNA molecule, respectively). In another embodiment, an adapter may be a Y-adapter. In another embodiment, an adapter may itself be composed of two distinct oligonucleotide molecules that are base paired with each other. As would be apparent, a ligatable end of an adapter may be designed to be compatible with overhangs made by cleavage by a restriction enzyme, or it may have blunt ends or a 5′ T overhang. The term “adapter” refers to double-stranded as well as single-stranded molecules. An adapter can be DNA or RNA, or a mixture of the two. An adapter containing RNA may be cleavable by RNase treatment or by alkaline hydrolysis. An adapter may be 15 to 100 bases, e.g., 50 to 70 bases, although adapters outside of this range are envisioned.

The term “adapter-ligated,” as used herein, refers to a nucleic acid that has been ligated to an adapter. The adapter can be ligated to a 5′ end and/or a 3′ end of a nucleic acid molecule. As used herein, the term “adding adapter sequences” refers to the act of adding an adapter sequence to the end of fragments in a sample. This may be done by filling in the ends of the fragments using a polymerase, adding an A tail, and then ligating an adapter comprising a T overhang onto the A-tailed fragments. Adapters are usually ligated to a DNA duplex using a ligase, while with RNA, adapters are covalently or otherwise attached to at least one end of a cDNA duplex preferably in the absence of a ligase.

The term “asymmetric adapter”, as used herein, refers to an adapter that, when ligated to both ends of a double stranded nucleic acid fragment, will lead to a top strand that contains a 5′ tag sequence that is not the same as or complementary to the tag sequence at the 3′ end. Examples of asymmetric adapters are described in U.S. Pat. Nos. 5,712,126 and 6,372,434 to Weissman et al., and International Patent Publication No. WO 2009/032167 to Bignell et al. An asymmetrically tagged fragment can be amplified by two primers: a first primer that hybridizes to a first tag sequence added to the 3′ end of a strand; and a second primer that hybridizes to the complement of a second tag sequence added to the 5′ end of a strand. Y-adapters and hairpin adapters (which can be cleaved, after ligation, to produce a “Y-adapter”) are examples of asymmetric adapters.

The term “Y-adapter” refers to an adapter that contains: a double-stranded region and a single-stranded region in which the opposing sequences are not complementary. The end of the double-stranded region can be joined to target molecules such as double-stranded fragments of genomic DNA, e.g., by ligation or a transposase-catalyzed reaction. Each strand of an adapter-tagged double-stranded DNA that has been ligated to a Y-adapter is asymmetrically tagged in that it has the sequence of one strand of the Y-adapter at one end and the other strand of the Y-adapter at the other end. Amplification of nucleic acid molecules that have been joined to Y-adapters at both ends results in an asymmetrically tagged nucleic acid, i.e., a nucleic acid that has a 5′ end containing one tag sequence and a 3′ end that has another tag sequence.

The term “hairpin adapter” refers to an adapter that is in the form of a hairpin. In one embodiment, after ligation the hairpin loop can be cleaved to produce strands that have non-complementary tags on the ends. In some cases, the loop of a hairpin adapter may contain a uracil residue, and the loop can be cleaved using uracil DNA glycosylase and endonuclease VIII, although other methods are known.

The term “adapter-ligated sample”, as used herein, refers to a sample that has been ligated to an adapter. As would be understood given the definitions above, a sample that has been ligated to an asymmetric adapter contains strands that have non-complementary sequences at the 5′ and 3′ ends.

The term “amplifying” as used herein refers to generating one or more copies, or “amplicons,” of a template nucleic acid, such as may be carried out using any suitable nucleic acid amplification technique, such as technology, such as PCR, NASBA, TMA, and SDA.

The terms “enrich” and “enrichment” refer to a partial purification of template molecules that have a certain feature (e.g., nucleic acids that contain 5-hydroxymethylcytosine) from analytes that do not have the feature (e.g., nucleic acids that do not contain hydroxymethylcytosine). Enrichment typically increases the concentration of the analytes that have the feature by at least 2-fold, at least 5-fold or at least 10-fold relative to the analytes that do not have the feature. After enrichment, at least 10%, at least 20%, at least 50%, at least 80% or at least 90% of the analytes in a sample may have the feature used for enrichment. For example, at least 10%, at least 20%, at least 50%, at least 80% or at least 90% of the nucleic acid molecules in an enriched composition may contain a strand having one or more hydroxymethylcytosines that have been modified to contain a capture tag.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained.

The terms “next-generation sequencing” (NGS) or “high-throughput sequencing”, as used herein, refer to the so-called parallelized sequencing-by-synthesis or sequencing-by-ligation platforms currently employed by Illumina, Life Technologies, Roche, etc. Next-generation sequencing methods may also include nanopore sequencing methods such as that commercialized by Oxford Nanopore Technologies, electronic detection methods such as Ion Torrent technology commercialized by Life Technologies, and single-molecule fluorescence-based methods such as that commercialized by Pacific Biosciences.

The term “read” as used herein refers to the raw or processed output of sequencing systems, such as massively parallel sequencing. In some embodiments, the output of the methods described herein is reads. In some embodiments, these reads may need to be trimmed, filtered, and aligned, resulting in raw reads, trimmed reads, aligned reads.

A “Unique Feature Identifier” (UFI) sequence refers to a relatively short nucleic acid sequence that serves to identify a feature of a nucleic acid molecule. Nucleic acid template molecules and amplicons thereof that contain a UFI are sometimes referred to herein as “barcoded” template molecules or amplicons. Examples of UFI sequence types include, without limitation, the following:

A “source identifier sequence” (or “source UFI” or “source barcode”) identifies the biological sample (or other source) of origin. That is, each DNA molecule in a single sample is tagged with the same source identifier sequence, thus allowing the mixing of samples prior to sequencing. These UFIs may also be characterized as a “sample identifier sequence,” a “sample UFI,” or “sample barcode.”

A “fragment identifier sequence” (or “fragment UFI” or “fragment barcode”): In a nucleic acid sample in which nucleic acids have been fragmented, each fragment in a sample is barcoded with a corresponding fragment identifier sequence. Sequence reads that have non-overlapping fragment identifier sequences represent different original nucleic acid template molecules, while reads that have the same fragment identifier sequences, or substantially overlapping fragment identifier sequences, likely represent fragments of the same template molecule. The unique feature identified here is the template nucleic acid molecule from which a fragment derives.

A “strand identifier sequence” (or “strand UFI” or “strand barcode”) independently tags each of the two strands of a DNA duplex, so that the strand from which a read originates can be determined, i.e., as the W strand or the C strand.

A “5hmC identifier sequence” (or “5hmC barcode”) identifies DNA fragments originating from 5hmC-containing cell-free DNA template molecules in a sample, i.e., “hydroxymethylated” DNA.

A “5mC identifier sequence” (or “5mC barcode”) identifies DNA fragments originating from 5mC-containing cell-free DNA template molecules that do not contain 5hmC.

A “molecular UFI sequence” (or “molecular barcode”) is appended to every nucleic acid template molecule in a sample, and is random, such that, providing the UFI sequence is of sufficient length, every nucleic acid template molecule is attached to a unique UFI sequence. Molecular UFI sequences, as is known in the art, can be used to account for and offset amplification and sequencer errors, allow a user to track duplicates and remove them from downstream analysis, and enable molecular counting, and, in turn, the determination of an analyte concentration. See, e.g., Casbon et al. (2011) Nuc. Acids Res. 39(12):1-8. The “unique feature” here is the identity of the nucleic acid template molecules.

In some embodiments, a UFI may have a length in the range of from 1 to about 35 nucleotides, e.g., from 3 to 30 nucleotides, 4 to 25 nucleotides, or 6 to 20 nucleotides. In certain cases, the UFI may be error-detecting and/or error-correcting, meaning that even if there is an error (e.g., if the sequence of the molecular barcode is mis-synthesized, mis-read or distorted during any of the various processing steps leading up to the determination of the molecular barcode sequence) then the code can still be interpreted correctly. The use of error-correcting sequences is described in the literature (e.g., in U.S. Patent Publication Nos. U.S. 2010/0323348 to Hamati et al. and U.S. 2009/0105959 to Braverman et al., both of which are incorporated herein by reference).

The oligonucleotides that serve as UFI sequences herein may be incorporated into DNA molecule using any effective means, where “incorporated into” is used interchangeably herein with “added to” and “appended to,” insofar as the UFI can be provided at the end of a DNA molecule, near the end of a DNA molecule, or within a DNA molecule. For example, multiple UFIs can be end-ligated to DNA using a selected ligase, in which case only the final UFI is at the “end” of the molecule. In addition, in the proximity extension assay and histone modification methods described in detail infra, the UFI may be contained within the nucleic acid tail of a proximity probe, at the end of the nucleic acid tail of a proximity probe, or within the hybridized region generated upon the binding of probes to the protein target.

More generally, the term “detection” is used interchangeably with the terms “determining,” “measuring,” “evaluating,” “assessing,” “assaying,” and “analyzing,” to refer to any form of measurement, and include determining if an element is present or not. These terms include both quantitative and/or qualitative determinations. Assessing may be relative or absolute. “Assessing the presence of” thus includes determining the amount of a moiety present, as well as determining whether it is present or absent. Assessing the level at a hydroxymethylation biomarker locus refers to a determination of the degree of hydroxymethylation at that locus.

“Accuracy” refers to the degree of conformity of a measured or calculated quantity (a test reported value) to its accurate (or true) value. Clinical accuracy relates to the proportion of true outcomes (true positives (TP) or true negatives (TN) versus misclassified outcomes (false positives (FP) or false negatives (FN), and may be stated as a sensitivity, specificity, positive predictive values (PPV) or negative predictive values (NPV), or as a likelihood, or odds ratio, among other measures.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Clinical parameters” encompass all non-sample biomarkers of subject health status or other characteristics, such as, without limitation, lesion size; lesion location; presence or absence of pancreatic inflammation; presence or absence of other symptoms; patient age; weight; jaundice; gender; ethnicity; family history; genetic mutations; diabetes mellitus (including Type I and Type II diabetes); physical activity; diet; pro-inflammatory cytokine levels; and smoking status of the patient.

A “formula,” “algorithm,” or “model” is any mathematical equation, algorithmic, analytical or programmed process, or statistical technique that takes one or more continuous or categorical inputs and calculates an output value, sometimes referred to as a “probability score” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining hydroxymethylation levels at various biomarker loci and clinical parameters, optionally in further combination with other factors (e.g., non-hydroxymethylation biomarkers), are linear and non-linear equations and statistical classification analyses to determine the relationship between hydroxymethylation levels at the biomarker loci detected in a patient sample and the patient's risk of having or developing pancreatic cancer. In panel and combination construction, of particular interest are structural and syntactic statistical classification algorithms, and methods of risk index construction, utilizing pattern recognition and machine learning features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Many such algorithmic techniques have been further implemented to perform both feature (loci) selection and regularization, such as in ridge regression, lasso, and elastic net, among others. Other techniques may be used in survival and time to event hazard analysis, including Cox, Weibull, Kaplan-Meier and Greenwood models well known to those of skill in the art. Many of these techniques are useful either combined with a hydroxymethylation biomarker selection technique, such as forward selection, backwards selection, or stepwise selection, complete enumeration of all potential biomarker sets, or panels, of a given size, genetic algorithms, or they may themselves include biomarker selection methodologies. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art.

“Risk,” in the context of the present invention, relates to the probability that an event will occur over a specific time period, as in the development of pancreatic cancer, and can mean a subject's “absolute” risk or “relative” risk. Absolute risk can be measured with reference to index values developed from statistically valid historical cohorts that have been followed for the relevant time period; an example of absolute risk herein is knowledge of the outcome of a pancreatic biopsy following surgical resection, Relative risk refers to the ratio of absolute risks of a subject compared either to the absolute risks of low risk cohorts.

“Risk evaluation” or “evaluation of risk” in the context of the present invention encompasses making a prediction of the probability, odds, or likelihood that an event or disease state may occur, the rate of occurrence of the event or conversion from one state to another, i.e., from an apparently benign pancreatic lesion to a cancerous lesion, and the like. The methods of the present invention may be used to make continuous or categorical measurements of the risk of conversion of an apparently benign pancreatic lesion to a cancerous lesion. In the categorical scenario, the invention can be used to discriminate between normal and other subject cohorts at higher risk for developing pancreatic cancer. In other embodiments, the present invention may be used so as to discriminate those at risk for developing pancreatic cancer from those having pancreatic cancer, or those likely to respond well to a particular treatment from those who are not. Such differing uses may require different hydroxymethylation biomarker combinations and individualized panels, mathematical algorithms, and/or cut-off points, but be subject to the same measurements of accuracy and performance for the respective intended use.

A “hydroxymethylation level” or “hydroxymethylation state” is the extent of hydroxymethylation within a hydroxymethylation biomarker locus. The extent of hydroxymethylation is normally measured as hydroxymethylation density, e.g., the ratio of 5hmC residues to total cytosines, both modified and unmodified, within a nucleic acid region. Other measures of hydroxymethylation density are also possible, e.g., the ratio of 5hmC residues to total nucleotides in a nucleic acid region.

A “hydroxymethylation profile” or “hydroxymethylation signature” refers to a data set that comprises the hydroxymethylation level at each of a plurality of hydroxymethylation biomarker loci. The hydroxymethylation profile may be a reference hydroxymethylation profile that comprises composite a hydroxymethylation profile for a population of individuals with at least one shared characteristic, as explained infra. The hydroxymethylation profile may also be a patient hydroxymethylation profile, constructed from the measurement of hydroxymethylation levels at each of a plurality of hydroxymethylation biomarker sites.

A “reference hydroxymethylation profile” thus refers to a data set representing the hydroxymethylation level of each of a plurality of hydroxymethylation biomarkers, where the data set is a composite of hydroxymethylation profiles of a plurality of individuals having at least one shared characteristic, e.g., individuals who have had a pancreatic lesion identified in an imaging scan, individuals who have not had a pancreatic lesion identified in an imaging scan, individuals who have not had pancreatic cancer, individuals with chronic pancreatitis, and the like.

The “hydroxymethylation biomarkers” herein comprise loci selected for their relevance to pancreatic cancer, particularly an exocrine pancreatic cancer such as PDAC. By “relevance” is meant that a hydroxymethylation biomarker locus, alone or in combination with one or more other hydroxymethylation biomarker loci, tends to exhibit an increase or decrease in hydroxymethylation in a manner that correlates with the risk, presence, absence, type, size, stage, invasiveness, grade, location, diagnosis, prognosis, outcome, and/or or likelihood of treatment responsiveness of pancreatic cancer, including the determinations of any of steps (a) through (j) in the preceding section.

The term “locus” in the preceding paragraph and throughout this application refers to a site on a nucleic acid molecule, wherein the nucleic acid molecule may be single-stranded or double-stranded, and further wherein an individual locus (or multiple “loci”) may be of any length, thus including a single CpG site as well as a full-length gene, or across larger features such as topologically associated domains, including when several such loci are aggregated into groups such as related sequence motifs, other homologies or functional characteristics (regardless of their adjacency or topological relationship). The loci herein may be contained within a gene body; within an annotation feature outside of the gene body, such as a promoter, an enhancer, a transcription initiation site, a transcription stop site, or a DNA binding site, or a combination thereof; or within an untranslated region, or “UTR” (including 3′UTRs and 5′UTRs). DNA binding sites that may contain one or more reference loci include, by way of example, silenced regions, transcription factor binding sites, transcription repressor binding sites, and CTCF binding sites (transposon repeat regions). Reference loci within CTCF binding sites are of particular interest, insofar as the CTCF gene codes for transcriptional repressor CTCF (also known as 11-zinc finger protein or CCCTC-binding factor), which in turn is involved in many cellular processes, including transcription regulation and regulation of chromatin architecture. See, for example, Juan et al. (2016) Cell Reports 14(5): 1246-1257; and Escedi et al. (2018) Epigenomes 2(1):3.

It should be noted that some of the individual hydroxymethylation biomarkers disclosed herein may not have significant individual significance in the evaluation of a pancreatic lesion, but when used in combination with other hydroxymethylation biomarkers disclosed herein and clinical parameters impacting on the evaluation and monitoring of a pancreatic lesion, optionally further combined with one or more other types of biomarkers and/or patient-specific risk factors, become significant in discriminating as a method of the invention requires, e.g., between a subject who has pancreatic cancer and a subject who does not have pancreatic cancer, or between a subject who is likely to develop pancreatic cancer and a subject who is not likely to develop pancreatic cancer, etc. The methods of the present invention provide an improvement over currently available methods of evaluating the risk that a subject has pancreatic cancer or is likely to develop pancreatic cancer, by using the biomarkers defined herein. To the extent that other biomarker pathway participants (i.e., other biomarker participants in common pathways with those biomarkers contained within the list of hydroxymethylation biomarkers herein are also relevant pathway participants in the subject pancreatic conditions, they may be functional equivalents to the hydroxymethylation biomarkers thus far disclosed. Furthermore, other unlisted hydroxymethylation biomarkers will be very highly correlated with the individual hydroxymethylation biomarkers listed here (for the purpose of this application, any two variables will be considered to be “very highly correlated” when they have a Coefficient of Determination (R2) of 0.5 or greater). The present invention encompasses such functional and statistical equivalents to the aforementioned hydroxymethylation biomarkers. Furthermore, the statistical utility of such additional hydroxymethylation biomarkers is substantially dependent on the cross-correlation between multiple biomarkers and any new biomarkers will often be required to operate within a panel in order to elaborate the meaning of the underlying biology.

The term “correlate” as used herein in reference to a variable (e.g., a value, a set of values, a disease state, a risk associated with the disease state, or the like) is a measure of the extent to which two or more variables fluctuate together. A positive correlation indicates the extent to which those variables increase or decrease in parallel. One example of a positive correlation is the relationship between a hydroxymethylation level at a hydroxymethylation biomarker locus, on the one hand, and the risk of developing pancreatic cancer, on the other, when the hydroxymethylation level increases as the risk of developing cancer increases. Conversely, a negative correlation would exist when the hydroxymethylation level biomarker at a hydroxymethylation biomarker locus decreases as the risk of developing cancer increases.

The term “pancreatic cancer” herein refers to an exocrine pancreatic cancer, particularly PDAC.

The present invention relates, in part, to the discovery that certain biological markers, particularly epigenetic markers relating to DNA hydroxymethylation, correlate in some way with pancreatic cancer, particularly an exocrine cancer such as PDAC. The methods involve measuring the hydroxymethylation level at each of a plurality of hydroxymethylation biomarker loci to generate a hydroxymethylation profile for a patient, and then comparing the patient's hydroxymethylation profile to a reference hydroxymethylation profile, at each locus. The biomarkers are differentially hydroxymethylated in subjects who have pancreatic cancer or are at risk of developing pancreatic cancer, particularly PDAC or another exocrine pancreatic cancer.

In some embodiments, the invention enables the determination of the risk that a pancreatic lesion observed with an imaging scan, i.e., an identified pancreatic lesion, is cancerous; the risk that an identified noncancerous pancreatic lesion will become cancerous; the likelihood that a particular therapy for treating a subject with pancreatic cancer will be effective; the risk that a subject without an identified pancreatic lesion will, at some point, develop a pancreatic lesion, as well as the risk of that lesion becoming cancerous.

The invention also enables a practitioner to determine the effectiveness of a therapy a subject is undergoing in connection with an identified pancreatic lesion; an increase or decrease in the risk that an identified pancreatic lesion will develop into cancer; an increase or decrease in the likelihood that a subject without an observed pancreatic lesion will develop a pancreatic lesion, and the risk of that lesion becoming cancerous; and a change in an identified pancreatic lesion, including a change in the size, stage, grade, or degree of invasiveness of a cancerous pancreatic lesion.

2. Determination of Hydroxymethylation Profile:

The present methods typically include a step in which a patient's hydroxymethylation profile is determined. The profile is generated by ascertaining the hydroxymethylation level at each of a plurality of hydroxymethylation biomarker loci, and assembling the data so obtained into a data set that serves as the hydroxymethylation profile. The hydroxymethylation biomarkers are differentially hydroxymethylated in subjects who, for instance, have PDAC or are at risk of developing PDAC, relative to a reference hydroxymethylation profile. That is, the biomarkers comprise regions of genomic DNA that are more susceptible to increases or decreases in hydroxymethylation level than other regions of the DNA, and that exhibit an increase or decrease in hydroxymethylation level in a manner that correlates with PDAC or a risk of developing PDAC.

In one embodiment, the invention provides a method for determining the presence of or likelihood of developing pancreatic ductal adenocarcinoma (PDAC) in a patient, where the method comprises: obtaining a cfDNA sample from the patient, such as from blood or pancreatic cyst fluid; sequencing the patient cfDNA in a manner that identifies 5hmC residues in the cfDNA; (c) mapping the identified 5hmC residues to each of a plurality of loci in a reference hydroxymethylation profile, wherein each locus serves as a hydroxymethylation biomarker comprising a gene feature selected from a 3′UTR, a TTS, an intron, and a promoter, the gene feature associated with a gene implicated in pancreatic development, a gene related to cancer development, or a gene established to exhibit hyper-hydroxymethylation in PDAC; (d) determining differences in extent and location between hydroxymethylation of the patient cfDNA and the reference hydroxymethylation profile at each locus; and (e) using the extent and location of the differences, calculating a probability score representing the likelihood that the patient has or is at risk of developing PDAC.

In a related embodiment, the invention provides a method for evaluating the risk that a pancreatic lesion identified on an imaging scan is cancerous. The imaging may be carried out using any suitable method, although cross-sectional imaging is preferred, e.g., using multi-detector row computed tomography (CT) or magnetic resonance imaging (MRI) with MR cholangiopancreatography (MRCP).

The first step in theses methods involves obtaining a cfDNA sample from a patient. Extraction of cfDNA from a blood sample (or pancreatic cyst fluid sample or the like) can be carried out using any suitable technique, for example using the commercially available kits referenced in the preceding section. The cfDNA is then enriched, so that the concentration of the cfDNA is substantially increased, a virtual necessity because of the very low levels of cfDNA normally obtained. A generally preferred enrichment technique is described in International Patent Publication WO 2017/176630 to Quake et al., incorporated herein by reference in its entirety: an affinity tag is appended to 5hmC residues in a sample of cfDNA, and the tagged DNA molecules are then selectively removed by bonding to a functionalized solid support. An illustrative example of the method, as described in Quake et al., involves initially modifying end-blunted, adaptor-ligated double-stranded DNA fragments in the cell-free sample to covalently attach biotin, as the affinity tag, to 5hmC residues. This may be carried out by selectively glucosylating 5hmC residues with uridine diphospho (UDP) glucose functionalized at the 6-position with an azide moiety, a step that is followed by a spontaneous 1,3-cycloaddition reaction with alkyne-functionalized biotin via a “click chemistry” reaction. The DNA fragments containing the biotinylated 5hmC residues are adapter-ligated dsDNA template molecules that can then be pulled down with a solid support functionalized with a biotin-binding protein (e.g., avidin or streptavidin) in the enrichment step.

The captured cfDNA is then amplified without having been releasing from the support, resulting in a plurality of amplicons. Any suitable amplification technique may be employed (e.g., PCR, NASBA, TMA, SDA) although PCR is preferred.

Next, the patient cfDNA is sequenced in a manner that identifies 5hmC residues, as will be explained infra. The identified 5hmC residues are mapped to each of a plurality of loci in a reference hydroxymethylation profile, where each locus serves as a hydroxymethylation biomarker comprising a gene feature that, as noted above, is selected from a 3′UTR, a TTS, an intron, and a promoter, the gene feature associated with a gene implicated in pancreatic development, a gene related to cancer development, or a gene established to exhibit hyper-hydroxymethylation in PDAC. Information regarding hydroxymethylation levels is thus deduced from the sequence reads obtained. That is, the sequence reads are analyzed to provide a quantitative determination of which sequences are hydroxymethylated in the cfDNA, and the level of hydroxymethylation. This may be done by, e.g., counting sequence reads or, alternatively, counting the number of original starting molecules, prior to amplification, based on their fragmentation breakpoint and/or whether they contain the same molecular UFI. The use of molecular UFI sequences (or “molecular barcodes” as they are sometimes called) in conjunction with other features of the fragments (e.g., the end sequences of the fragments, which define the breakpoints) to distinguish between the fragments is known. See Casbon (2011) Nucl. Acids Res. 22 e81 and Fu et al. (2011) Proc. Natl. Acad. Sci. USA 108: 9026-31), among others. Molecular barcodes are also described in U.S. Patent Publication Nos. 2015/0044687, 2015/0024950, and 2014/0227705, and in U.S. Pat. Nos. 8,835,358 and 7,537,897, as well as a variety of other publications.

A molecular UFI sequence is preferably incorporated into the adapters that are end-ligated to the cfDNA following extraction thereof. The adapters may be constructed so as to comprise an additional UFI sequence, e.g., a sample UFI sequence, a strand-identifier UFI sequence, or both.

Other methods of ascertaining the hydroxymethylation profile of DNA in the cell-free nucleic sample are described in International Patent Publication WO 2019/160994 A1 to Arensdorf et al. for “Methods for the Epigenetic Analysis of DNA, particularly Cell-Free DNA”; in co-pending U.S. patent application Ser. Nos. 16/275,237 and 17/118,234 to Arensdorf et al.; and in Liu et al. (2019) Nature Biotech. 37: 424-29, all of which are incorporated by reference herein. These references are also useful in conjunction with an embodiment of the invention in which a patient's cfDNA methylation profile is identified in addition to the patient's cfDNA hydroxymethylation profile.

Mapping the identified 5hmC residues to each of a plurality of loci in a reference hydroxymethylation profile enables the determination of differences between the hydroxymethylation profile of the patient and the reference hydroxymethylation profile, with respect to both the extent and the location of those differences.

The selected loci in the above described method are hydroxymethylation biomarkers, i.e., loci that have been identified herein as differentially hydroxymethylated in a manner that relates to the presence, absence, or risk of PDAC. Certain hydroxymethylation biomarkers that are particularly useful in conjunction with the present methods, as established in Example 1, include, without limitation, those set forth in Table 1 (along with chromosome location):

TABLE 1

| Gene | Location |
|---|---|
| ADARB2-AS1 | chr10 |
| ANKRD36B | chr02 |
| ASAH2B | chr10 |
| ATG4B | chr02 |
| ATP8B1 | chr18 |
| BOLA1 | chr01 |
| C11orf88 | chr11 |
| C17orf97 | chr17 |
| C1orf170 | chr01 |
| C3orf36 | #N/A |
| C8orf74 | chr08 |
| CAMSAP2 | chr01 |
| CCDC54 | chr03 |
| CCDC59 | chr12 |
| CKAP2 | chr13 |
| CLK2P | #N/A |
| CRTC1 | chr19 |
| CSRP2 | chr12 |
| CYB5D1 | chr17 |
| DNAJC27 | #N/A |
| DYNAP | #N/A |
| FAM166A | #N/A |
| FAM188B | chr07 |
| FAM196A | chr10 |
| FAM86JP | #N/A |
| FAT4 | chr04 |
| FBXO5 | chr06 |
| FGF2 | chr04 |
| FUT2 | chr19 |
| GAS2L2 | chr17 |
| GAS6 | #N/A |
| GGACT | chr13 |
| GLRX5 | chr14 |
| GPX1 | #N/A |
| GPX5 | chr06 |
| HBD | chr11 |
| HLA-A | chr06 |
| HTR1F | chr03 |
| IL36G | #N/A |
| KANSL1 | #N/A |
| KCNH6 | #N/A |
| KCTD15 | #N/A |
| KLHL38 | #N/A |
| KLK2 | #N/A |
| KRT6B | chr12 |
| LAMC1 | chr01 |
| LGALS14 | chr19 |
| LGALS8-AS1 | #N/A |
| LIFR | chr05 |
| LINC00266-1 | #N/A |
| LINC00310 | chr21 |
| LOC100130452 | chr02 |
| LOC100130557 | #N/A |
| LOC100130894 | #N/A |
| LOC100288778 | #N/A |
| LOC100505633 | #N/A |
| LOC100505648 | chr15 |
| LOC100505738 | #N/A |
| LOC100652909 | chr19 |
| LOC389033 | #N/A |
| LOC90784 | #N/A |
| LRRC37A2 | #N/A |
| MED11 | chr17 |
| MRPL23-AS1 | chr11 |
| NAT8L | chr04 |
| NEUROD1 | #N/A |
| NEUROG2 | chr04 |
| NME5 | chr05 |
| NOMO3 | chr16 |
| NPRL2 | chr03 |
| NXN | chr17 |
| ODF3L1 | #N/A |
| ODF3L2 | chr19 |
| OSCP1 | chr01 |
| PARD6G | chr18 |
| PGAM1 | #N/A |
| PLA2G2E | #N/A |
| PLSCR4 | #N/A |
| PPAP2A | chr05 |
| PPP1R15A | chr19 |
| PPP1R3E | #N/A |
| RASL10B | chr17 |
| REXO1L1 | #N/A |
| RIMBP3 | #N/A |
| RNF126P1 | chr17 |
| RNU6-76 | chr16 |
| RPP25 | chr15 |
| RPS27 | #N/A |
| SH3PXD2B | #N/A |
| SHISA4 | #N/A |
| SLC25A38 | #N/A |
| SLC4A1 | #N/A |
| SLCO5A1 | #N/A |
| SPDEF | chr06 |
| SRSF6 | #N/A |
| STRA6 | #N/A |
| SYNM | chr15 |
| TBCB | chr19 |
| TDRD6 | #N/A |
| TEX26 | #N/A |
| TMEM253 | #N/A |
| TNFSF13B | #N/A |
| TTC14 | #N/A |
| TUBA4A | #N/A |
| UBB | chr17 |
| VAMP8 | chr02 |
| VGLL2 | #N/A |
| WASH2P | #N/A |
| WNT9B | #N/A |
| XBP1 | chr22 |
| ZNF789 | #N/A |

While the experimental work documented in Example 1 identified thousands of genes in which 5hmC is differentially expressed, the above group represents a stringently filtered set of the most significant genes using elastic net regularization (glmnetF) or lasso regularization (glmnet2F). The above 111 genes were found to exhibit biology related to pancreatic development (GATA4, GATA6, PROX1, ONECUT2) and/or cancer development (YAP1, TEAD, PROX1, ONECUT2, ONECUT1, IGF1 and IGF2), as explained in Example 1 herein. Table 2 indicates those genes identified using glmnetF and Table 3 indicates those genes identified using glmnet2F:

TABLE 2

| Gene | Location |
|---|---|
| ADARB2-AS1 | chr10 |
| ANKRD36B | chr02 |
| ATG4B | chr02 |
| ATP8B1 | chr18 |
| BOLA1 | chr01 |
| C11orf88 | chr11 |
| C17orf97 | chr17 |
| C1orf170 | chr01 |
| C3orf36 | #N/A |
| C8orf74 | chr08 |
| CAMSAP2 | chr01 |
| CCDC54 | chr03 |
| CCDC59 | chr12 |
| CKAP2 | chr13 |
| CLK2P | #N/A |
| CRTC1 | chr19 |
| CSRP2 | chr12 |
| CYB5D1 | chr17 |
| DNAJC27 | #N/A |
| DYNAP | #N/A |
| FAM166A | #N/A |
| FAM188B | chr07 |
| FAM196A | chr10 |
| FAM86JP | #N/A |
| FAT4 | chr04 |
| FBXO5 | chr06 |
| FGF2 | chr04 |
| FUT2 | chr19 |
| GAS2L2 | chr17 |
| GAS6 | #N/A |
| GGACT | chr13 |
| GLRX5 | chr14 |
| GPX1 | #N/A |
| GPX5 | chr06 |
| HBD | chr11 |
| HLA-A | chr06 |
| HTR1F | chr03 |
| IL36G | #N/A |
| KANSL1 | #N/A |
| KCNH6 | #N/A |
| KCTD15 | #N/A |
| KLHL38 | #N/A |
| KLK2 | #N/A |
| KRT6B | chr12 |
| LAMC1 | chr01 |
| LGALS14 | chr19 |
| LGALS8-AS1 | #N/A |
| LIFR | chr05 |
| LINC00266-1 | #N/A |
| LINC00310 | chr21 |
| LOC100130557 | #N/A |
| LOC100130894 | #N/A |
| LOC100288778 | #N/A |
| LOC100505633 | #N/A |
| LOC100505648 | chr15 |
| LOC100505738 | #N/A |
| LOC100652909 | chr19 |
| LOC389033 | #N/A |
| LOC90784 | #N/A |
| LRRC37A2 | #N/A |
| MED11 | chr17 |
| MRPL23-AS1 | chr11 |
| NAT8L | chr04 |
| NEUROD1 | #N/A |
| NEUROG2 | chr04 |
| NME5 | chr05 |
| NOMO3 | chr16 |
| NPRL2 | chr03 |
| NXN | chr17 |
| ODF3L1 | #N/A |
| ODF3L2 | chr19 |
| OSCP1 | chr01 |
| PARD6G | chr18 |
| PGAM1 | #N/A |
| PLA2G2E | #N/A |
| PLSCR4 | #N/A |
| PPAP2A | chr05 |
| PPP1R15A | chr19 |
| PPP1R3E | #N/A |
| RASL10B | chr17 |
| REXO1L1 | #N/A |
| RIMBP3 | #N/A |
| RNF126P1 | chr17 |
| RNU6-76 | chr16 |
| RPP25 | chr15 |
| RPS27 | #N/A |
| SH3PXD2B | #N/A |
| SHISA4 | #N/A |
| SLC25A38 | #N/A |
| SLC4A1 | #N/A |
| SLCO5A1 | #N/A |
| SPDEF | chr06 |
| SRSF6 | #N/A |
| STRA6 | #N/A |
| SYNM | chr15 |
| TBCB | chr19 |
| TDRD6 | #N/A |
| TEX26 | #N/A |
| TMEM253 | #N/A |
| TNFSF13B | #N/A |
| TTC14 | #N/A |
| TUBA4A | #N/A |
| UBB | chr17 |
| VAMP8 | chr02 |
| VGLL2 | #N/A |
| WASH2P | #N/A |
| WNT9B | #N/A |
| XBP1 | chr22 |
| ZNF789 | #N/A |

TABLE 3

| Gene | Location |
|---|---|
| ASAH2B | chr10 |
| BOLA1 | chr01 |
| C11orf88 | chr11 |
| C17orf97 | chr17 |
| C3orf36 | #N/A |
| CCDC54 | chr03 |
| CKAP2 | chr13 |
| CLK2P | #N/A |
| DNAJC27 | chr02 |
| DYNAP | #N/A |
| FAM166A | chr09 |
| FGF2 | chr04 |
| FUT2 | chr19 |
| GAS6 | #N/A |
| GGACT | chr13 |
| GPX1 | #N/A |
| GPX5 | chr06 |
| IL36G | #N/A |
| KCNH6 | chr17 |
| KCTD15 | chr19 |
| KLK2 | #N/A |
| KRT6B | chr12 |
| LGALS14 | chr19 |
| LGALS8-AS1 | #N/A |
| LIFR | chr05 |
| LINC00266-1 | #N/A |
| LINC00310 | chr21 |
| LOC100130452 | chr02 |
| LOC100130557 | #N/A |
| LOC100288778 | #N/A |
| LOC100505648 | chr15 |
| MED11 | chr17 |
| NAT8L | chr04 |
| NEUROG2 | chr04 |
| NME5 | chr05 |
| NOMO3 | chr16 |
| NPRL2 | chr03 |
| NXN | chr17 |
| ODF3L1 | #N/A |
| PPP1R3E | #N/A |
| RASL10B | chr17 |
| RNF126P1 | chr17 |
| SH3PXD2B | chr05 |
| SLC25A38 | chr03 |
| TNFSF13B | chr13 |
| VAMP8 | chr02 |
| VGLL2 | #N/A |

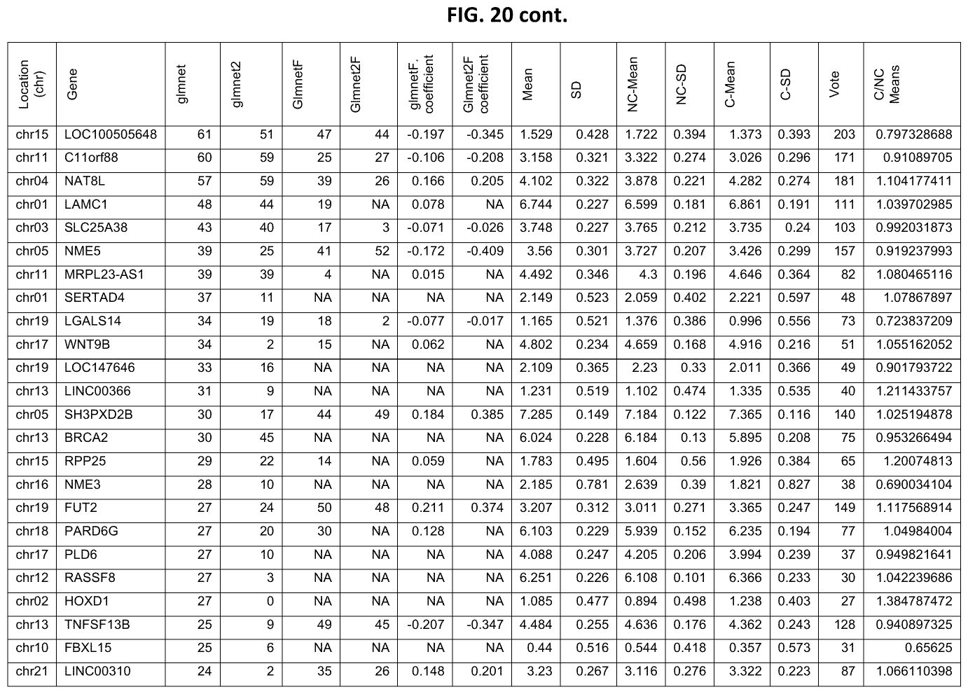

Other hydroxymethylation biomarkers that are useful in conjunction with the present methods are the 611 genes set forth in FIG. 21, along with location and glmnet value (identified using Study Group 2 in Example 2). Hydroxymethylation biomarkers within this group that may be of particular interest are the 41 biomarkers set forth in Table 4, again along with location and glmnet value (from Study Group 3 in Example 3):

TABLE 4

| Gene | Location | Glmnet |
|---|---|---|
| LINC00457 | chr13~−~35009590~35214822~ | 100 |
| CERS3 | chr15~−~100940599~101084925~ | 67 |
| LOC285629 | chr05~−~160358785~160365633~ | 65 |
| RHOJ | chr14~+~63671101~63760230~ | 57 |
| GP2 | chr16~−~20321810~20338835~ | 56 |
| SFRP1 | chr08~−~41119475~41166990~ | 56 |
| LY6G6F | chr06~+~31674683~31678372~ | 51 |
| HOXA4 | chr07~−~27168125~27170399~ | 50 |
| MYOCD | chr17~+~12569206~12670651~ | 46 |
| C14orf64 | chr14~−~98391946~98444461~ | 45 |
| PDE10A | chr06~−~165740775~166075588~ | 42 |
| PTCRA | chr06~+~42883726~42893575~ | 42 |
| UCP3 | chr11~−~73711325~73720282~ | 41 |
| NTRK2 | chr09~+~87283465~87638505~ | 36 |
| GABRGR3 | chr15~+~27216428~27778373~ | 33 |
| FBXL7 | chr05~+~15500304~15939900~ | 31 |
| LOC100128714 | chr15~+~26147506~26298267~ | 23 |
| LOC151171 | chr02~−~239419330~239464140~ | 23 |
| MIR5009 | chr21~−~28659821~29283529~ | 20 |
| HKR1 | chr19~+~37825579~37855357~ | 17 |
| ZNF573 | chr19~−~38229202~38270230~ | 16 |
| LINC00670 | chr17~+~12453284~12540504~ | 16 |
| FILIP1 | chr06~−~76017799~76203496~ | 16 |
| DCLK1 | chr13~−~36342788~36705514~ | 15 |
| OSGEP | chr14~−~20915206~20923267~ | 13 |
| BNC2 | chr09~−~16409500~16870786~ | 12 |
| ABCC12 | chr16~−~48116883~48180681~ | 11 |
| PCDH7 | chr04~+~30722029~31148423~ | 10 |
| ZNF2 | chr02~+~95831182~95850064~ | 10 |
| PDLIM1 | chr10~−~96997324~97050905~ | 9 |
| TSPAN33 | chr07~+~128784711~128809534~ | 9 |
| MRPS5 | chr02~−~95752951~95787754~ | 8 |
| C15orf53 | chr15~+~38988798~38992239~ | 7 |
| CAMK1G | chr01~+~209757044~209787284~ | 7 |
| TWIST2 | chr02~+~239756672~239832237~ | 6 |
| FGF9 | chr13~+~22245214~22278640~ | 5 |
| CCDC129 | chr07~+~31553684~31698334~ | 5 |
| ISLR2 | chr15~+~74421714~74429143~ | 4 |
| C17orf51 | chr17~−~21431570~21454941~ | 2 |
| ADAMTS9-AS2 | chr03~+~64670545~64997143~ | 2 |
| GABRA5 | chr15~+~27111865~27194357~ | 1 |

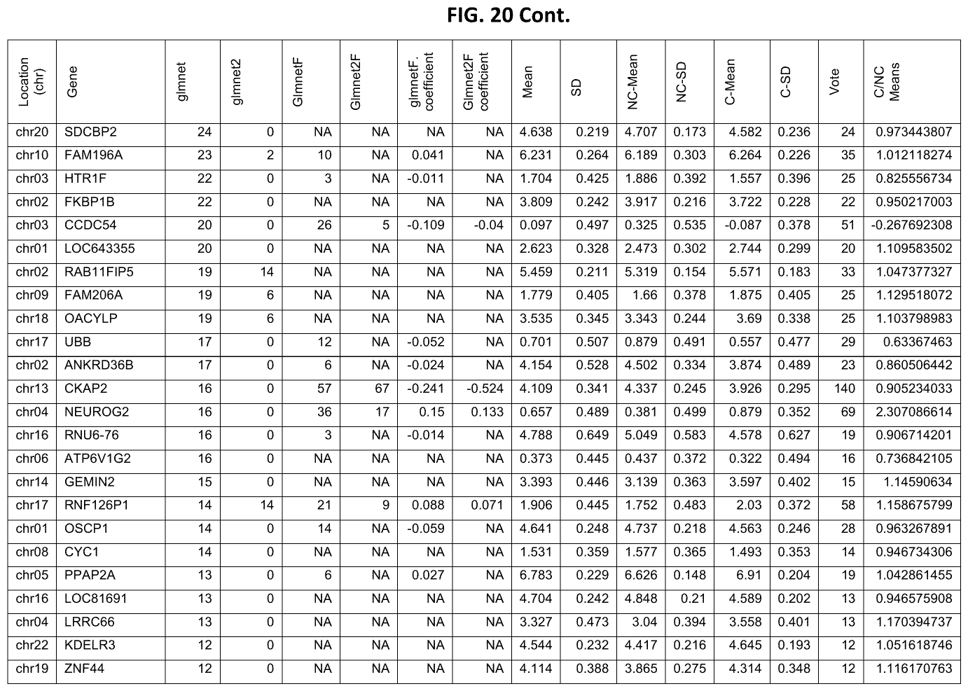

Also see FIG. 22, which provides detailed information regarding the 41 hydroxymethylation biomarkers of Table 4.

Additional hydroxymethylation biomarkers are found in the genes of Table 5:

TABLE 5

| Gene | Location (chr; termini) | | |
|---|---|---|---|
| SLFN14 | chr17 | 33875143 | 33885110 |
| SECISBP2 | chr9 | 91933411 | 91974561 |
| TSPAN33 | chr7 | 128784711 | 128809534 |
| ZAR1 | chr4 | 48492308 | 48496422 |
| VSTM2B | chr19 | 30017490 | 30055226 |
| C12orf73 | chr12 | 104343980 | 104350993 |
| LY6G6F | chr6 | 31674683 | 31678372 |
| KIAA1919 | chr6 | 111580481 | 111590261 |
| PPFIA4 | chr1 | 203020310 | 203047864 |
| UCP3 | chr11 | 73711325 | 73720282 |
| ZNF233 | chr19 | 44764032 | 44779470 |
| IZUMO2 | chr19 | 50655804 | 50666538 |
| TPO | chr2 | 1417232 | 1546499 |
| CD22 | chr19 | 35820071 | 35838264 |
| MSI2 | chr17 | 55333930 | 55757299 |
| LINC00457 | chr13 | 35009590 | 35214822 |
| PDE10A | chr6 | 165740775 | 166075588 |
| TPTE2 | chr13 | 19997018 | 20135714 |
| OR9Q1 | chr11 | 57791352 | 57949038 |
| MCHR2 | chr6 | 100367785 | 100442114 |
| RUNX1T1 | chr8 | 92967194 | 93115454 |
| RHOJ | chr14 | 63671101 | 63760230 |
| ZNF253 | chr19 | 19976713 | 20004293 |
| GABRB3 | chr15 | 26788693 | 27018935 |
| MYOCD | chr17 | 12569206 | 12670651 |
| LOC100505658 | chr5 | 140937877 | 140944827 |
| BAGE3 | chr21 | 11020841 | 11098925 |
| BAGE4 | chr21 | 11020841 | 11098925 |
| BAGE5 | chr21 | 11020841 | 11098925 |
| ADAMTS12 | chr5 | 33527286 | 33892124 |
| ZNF737 | chr19 | 20720797 | 20748626 |
| BAGE | chr21 | 11057795 | 11098937 |
| LOC283867 | chr16 | 65318401 | 65610203 |
| PCDH7 | chr4 | 30722029 | 31148423 |
| ZNF793 | chr19 | 37997840 | 38034239 |
| C14orf64 | chr14 | 98391946 | 98444461 |
| SFRP1 | chr8 | 41119475 | 41166990 |

One preferred method for sequencing the patient cfDNA in a manner that identifies 5hmC residues, i.e., for determining the hydroxymethylation profile of a patient, is described in International Patent Publication WO 2017/176630 to Quake et al., incorporated herein by reference. That method pertains to the detection of 5-hydroxymethylcytosine patterns in cell-free DNA within the context of a sequencing scheme. An affinity tag is appended to 5hmC residues in a sample of cell-free DNA, and the tagged DNA molecules are then enriched and sequenced, with the sequences of 5hmC-containing DNA fragments identified. An illustrative example of the method, as described in Quake et al., involves initially modifying end-blunted, adaptor-ligated double-stranded DNA fragments in the cell-free sample to covalently attach biotin, as the affinity tag, to 5hmC residues. This may be carried out by selectively glucosylating 5hmC residues with uridine diphospho (UDP) glucose functionalized at the 6-position with an azide moiety, a step that is followed by a spontaneous 1,3-cycloaddition reaction with alkyne-functionalized biotin via a “click chemistry” reaction, as described previously, in Section 5, with respect to 5hmC-containing capture sequences in adapters. The DNA fragments containing the biotinylated 5hmC residues are adapter-ligated dsDNA template molecules that can then be pulled down with streptavidin beads in an “enrichment” step.

Both targeted and non-sequencing detection approaches after enrichment may also be used to quantitate specific hydroxymethylation biomarkers and loci of interest, if genome-wide coverage through shotgun sequencing is not required or desirable (generally for cost reasons). For example, after 5hmC enrichment, targeted PCR amplicons covering only specific regions may be generated from the 5hmC-enriched templates and employed as a narrower genome coverage approach, and used as input to sequencing or detected directly.

When a smaller number of discrete loci are of interest, the combination of these post-enrichment approaches with target amplification may also be an efficient way to reduce the number of sequencing reads (and sequencing costs) required for each sample, enabling further sample multiplexing per sequencing run and further reducing the sequencing costs required for each sample). In non-sequencing approaches, quantitative PCR or even hybridization assays could themselves be used as the quantitative readouts of the hydroxymethylation biomarkers (e.g., using direct fluorescence nucleotide labeling and microarray or other substrate capture and binding); such approaches are well known in the art, and frequently scaled to hundreds or even thousands of short amplicons.

In the present process, a 5hmC UFI sequence is added to the termini of the pulled down adapter-ligated dsDNA template molecules, so that the after amplification, pooling, and sequencing, information regarding hydroxymethylation profile can be deduced from the sequence reads obtained. That is, the sequence reads are analyzed to provide a quantitative determination of which sequences are hydroxymethylated in the cfDNA. This may be done by, e.g., counting sequence reads or, alternatively, counting the number of original starting molecules, prior to amplification, based on their fragmentation breakpoint and/or whether they contain the same molecular UFI. The use of molecular UFI sequences (or “molecular barcodes” as they are sometimes called) in conjunction with other features of the fragments (e.g., the end sequences of the fragments, which define the breakpoints) to distinguish between the fragments is known. See Casbon (2011) Nucl. Acids Res. 22 e81 and Fu et al. (2011) Proc. Natl. Acad. Sci. USA 108: 9026-31), among others. Molecular barcodes are also described in U.S. Patent Publication Nos. 2015/0044687, 2015/0024950, and 2014/0227705, and in U.S. Pat. Nos. 8,835,358 and 7,537,897, as well as a variety of other publications.

Other methods of ascertaining the hydroxymethylation profile of DNA in the cell-free nucleic sample are described in International Patent Publication WO 2019/160994 A1 to Arensdorf et al. for “Methods for the Epigenetic Analysis of DNA, particularly Cell-Free DNA” and in U.S. Patent Publication No. 2017/0298422 to Song et al., previously incorporated by reference herein. These references are also useful in conjunction with an embodiment of the invention in which the present combined workflow process further includes the detection of a cfDNA methylation profile in addition to the cfDNA hydroxymethylation profile.

The Arensdorf et al. methodology described in WO 2019/160994, in the context of the present combined workflow process, can be implemented as follows:

Dual-Biotin Technique: After a cell-free nucleic acid sample has been extracted from a biological sample, with cfDNA having been adapter-ligated, 5hmC residues in the cfDNA are selectively labeled with an affinity tag, e.g., a biotin moiety as explained earlier herein. Biotinylation can be carried out by selective functionalization of 5hmC residues via βGT-catalyzed glucosylation with uridine diphosphoglucose-6-azide followed by a click chemistry reaction to covalently attach an alkyne-functionalized biotin moiety as explained previously. An avidin or streptavidin surface (e.g., in the form of streptavidin beads) is then used to pull out all of the dsDNA template molecules biotinylated at the 5hmC locations, which are then placed in a separate container for UFI sequence attachment during amplification. The remaining dsDNA template molecules in the supernatant are fragments that either have 5mC residues or have no modifications (the latter group including cDNA generated from cfRNA). A TET protein is then used to oxidize 5mC residues in the supernatant to 5hmC; in this case, a TET mutant protein is employed to ensure that oxidation of 5mC does not proceed beyond hydroxylation. Suitable TET mutant proteins for this purpose are described in Liu et al. (2017) Nature Chem. Bio. 13: 181-191, incorporated by reference herein. The βGT-catalyzed glucosylation followed by biotin functionalization is then repeated. The fragments so marked—biotinylated at each of the original 5mC locations—are pulled down with streptavidin beads. The bead-bound DNA fragments are then barcoded—with a UFI sequence than used in the first step, i.e., a 5mC UFI sequence—during amplification. Unmodified DNA fragments, i.e., fragments containing no modified cytosine residues, now remain in the supernatant. If desired, sequence-specific probes can be used to hybridize to unmethylated DNA strands. The hybridized complexes that result can be pulled out and tagged with a further UFI sequence during amplification, as before.

Pyridine Borane Methodology: This is an alternative to the dual biotin technique, and is also a bisulfite-free process. The method relies on the use of pyridine borane, or an alternative, equally effective organic borane, to convert 5-carboxylcytosine (5caC) and 5-formylcytosine (5fC)—both of which can be generated from 5mC and 5hmC—to dihydrouracil (DHU). As DHU residues are read as thymine (T), while 5mC and 5hmC are read as C, the difference between parallel sequence reads enables the determination of DHU locations, which in turn indicates the location of 5mC and 5hmC locations.

In one embodiment, the pyridine borane method enables the identification of 5hmC locations in adapter-ligated target DNA in a cell-free sample. Initially, target DNA is oxidized with an oxidizing reagent that converts 5hmC to 5caC or 5fC, where the oxidizing reagent selected does not affect 5mC. Oxidation may be carried out enzymatically, although chemical oxidizing reagents are preferred in this embodiment. Examples of suitable chemical oxidizing agents for use in carrying out the aforementioned conversion include, without limitation: a perruthenate anion in the form of an inorganic or organic perruthenate salt, including metal perruthenates such as potassium perruthenate (KRuO₄), tetraalkylammonium perruthenates such as tetrapropylammonium perruthenate (TPAP) and tetrabutylammonium perruthenate (TBAP), and polymer supported perruthenate (PSP); and inorganic peroxo compounds and compositions such as peroxotungstate or a copper (II) perchlorate/TEMPO combination. The modified DNA containing 5caC or 5fC in lieu of 5hmC is then treated with an organic borane effective to reduce, deaminate, and either decarboxylate or deformylate the oxidized 5hmC and provide DHU in place thereof. The DHU-containing DNA is amplified and sequence to provide 5hmC-indicative sequence reads, insofar as the sequence reads can be readily compared to standard sequence reads obtained for the target DNA, where the change from C in the standard sequence reads to a T in the 5hmC-indicative sequence reads indicates a 5hmC location.

In another embodiment, the pyridine borane methodology is used to identify 5mC locations in adapter-ligated target DNA in a cell-free sample. In this case, 5hmC residues in the target DNA are, at the outset, tagged with an affinity tag that enables removal of 5hmC-containing fragments from the sample. For instance, 5hmC residues may be selectively glucosylated with uridine diphospho (UDP) glucose functionalized at the 6-position with an azide moiety, a step that is followed by a spontaneous 1,3-cycloaddition reaction with alkyne-functionalized biotin via a “click chemistry” reaction. The resulting biotinylated DNA target molecules can then be separated from the sample with a solid support functionalized with a biotin-binding protein (e.g., avidin or streptavidin). Remaining DNA in the sample will contain 5mC, but not 5hmC. In the next step, 5mC residues in the remaining DNA are enzymatically oxidized to 5caC or 5fC, followed by treatment with pyridine borane to convert the oxidized 5mC residues to DHU. A preferred enzyme useful as the oxidizing agent is a Ten-Eleven Translocation Enzyme (TET) family enzyme or a “TET catalytically active fragment” as defined in U.S. Pat. No. 9,115,386, the disclosure of which is incorporated by reference herein. A preferred TET enzyme in this context is TET2; see Ito et al. (2011) Science 333(6047):1300-1303. Following amplification and sequencing, a comparison of the sequence reads obtained with the standard sequence reads for the target DNA indicates the location of 5mC residues in the sample DNA, as the change from C to T (resulting from DHU substitution for 5mC) indicates a 5mC location.

In a further embodiment, the pyridine borane technique can be implemented to detect the locations of both 5mC and 5hmC residues in a single cell-free DNA sample. The method involves, for a first fraction of a cell-free DNA sample comprising adapter-ligated target DNA,

(a) blocking 5hmC residues with a blocking reagent to yield blocked 5hmC residues;

(b) enzymatically oxidizing 5mC residues to provide oxidized 5mC residues selected from 5caC, 5fC, and combinations thereof;

(c) converting the oxidized 5mC residues to DHU by treatment with pyridine borane, thereby providing first fraction DNA comprising blocked 5hmC residues and DHU at 5mC locations; and

(d) amplifying and sequencing the first fraction DNA to provide first fraction sequence reads in which the blocked 5hmC residues read as C and DHU reads as T.

Glucosylation is effective as a blocking technique, in which case the blocking reagent may be β-glucosyltransferase and the resulting blocking group on the 5hmC residues is glucose.

For a second fraction of the same sample, the method further involves:

(e) oxidizing 5hmC residues with an oxidizing reagent effective to convert 5hmC residues to oxidized 5hmC residues without modifying 5mC residues, wherein the oxidized 5hmC residues are selected from 5caC, 5fC, and combinations thereof; and

(f) converting the oxidized 5hmC residues to DHU by treatment with pyridine borane, thereby providing second fraction DNA comprising unmodified 5mC residues and DHU at 5hmC locations;

(g) amplifying and sequencing the second fraction DNA to provide second fraction sequence reads in which the unmodified 5mC residues read as C and DHU reads as T; and

(h) comparing the first fraction sequence reads with the second fraction sequence reads to identify 5mC and 5hmC locations in the template DNA.

See, e.g., Liu et al. (2019) Nature Biotech. 37: 424-429.

The organic borane may be characterized as a complex of borane and a nitrogen-containing compound selected from nitrogen heterocycles and tertiary amines. The nitrogen heterocycle may be monocyclic, bicyclic, or polycyclic, but is typically monocyclic, in the form of a 5- or 6-membered ring that contains a nitrogen heteroatom and optionally one or more additional heteroatoms selected from N, O, and S. The nitrogen heterocycle may be aromatic or alicyclic. Preferred nitrogen heterocycles herein include 2-pyrroline, 2H-pyrrole, 1H-pyrrole, pyrazolidine, imidazolidine, 2-pyrazoline, 2-imidazoline, pyrazole, imidazole, 1,2,4-triazole, 1,2,4-triazole, pyridazine, pyrimidine, pyrazine, 1,2,4-triazine, and 1,3,5-triazine, any of which may be unsubstituted or substituted with one or more non-hydrogen substituents. Typical non-hydrogen substituents are alkyl groups, particularly lower alkyl groups, such as methyl, ethyl, n-propyl, isopropyl, n-butyl, isobutyl, t-butyl, and the like. Exemplary compounds include pyridine borane, 2-methylpyridine borane (also referred to as 2-picoline borane), and 5-ethyl-2-pyridine. Further information concerning these organic boranes and reaction thereof to convert oxidized 5mC residues to DHU may be found in the Arensdorf patent publication cited above.

Biotin/Native 5mC Enrichment Method: This is an alternative to the dual biotin technique, and begins with biotinylation of 5hmC residues in adapter-ligated DNA fragments, followed by avidin or streptavidin pull-down. Here, however, instead of modifying the methylated DNA that remains in the supernatant, an anti-5mC antibody or an MBD protein is used to capture and pull down native 5mC-containing fragments. This technique is less preferred herein, insofar as it does not result in the generation of dsDNA template molecules that can be amplified, pooled, and sequenced with other dsDNA template molecules deriving from the same sample.

The extent and location of the differences in patient cfDNA hydroxymethylation relative to the reference hydroxymethylation profile are then used to calculate a probability score representing the likelihood that the patient has PDAC or is at risk of developing PDAC.

In order to calculate the probability score, the methods of the invention include statistical analyses and mathematical modeling used to analyze high-dimensional and multimodal biomedical data, i.e., the data obtained using the present methods for comparing hydroxymethylation profiles. More specifically, the methods make use of one or more objective algorithms, models, and analytical methods that include mathematical analyses based on topographic, pattern-recognition based protocols, e.g., support vector machines (SVM), linear discriminant analysis (LDA), naive Bayes (NB), and K-nearest neighbor (KNN) protocols, as well as other supervised learning algorithms and models, such as Decision Tree, Perceptron, and regularized discriminant analysis (RDA), and similar models and algorithms well-known in the art (Gallant S I, “Perceptron-based learning algorithms,” Perceptron-based learning algorithms 1990; 1(2):179-91).

Statistical analyses include determining mean (M), e.g., geometric mean, standard deviations (SD), Geometric Fold Change (FC), and the like. Whether differences in hydroxymethylation levels are deemed significant may be determined by well-known statistical approaches, typically by designating a threshold for a particular statistical parameter, such as a threshold p-value (e.g., p<0.05),a threshold S-value (e.g., ±0.4, with S≤−0.4 or S>0.4), or other value, at which differences are deemed significant, for example when the level of biomarker hydroxymethylation in a hydroxymethylation profile is considered significantly increased or decreased, respectively, relative to the hydroxymethylation level at the same hydroxymethylation biomarker locus in a reference hydroxymethylation profile.

In one aspect, the methods of the invention apply the mathematical formulations, algorithms or models to distinguish between normal and cancerous samples, and between various sub-types, stages, and other aspects of disease or disease outcome. In another aspect, the methods are used for prediction, classification, prognosis, and treatment monitoring and design.

For the comparison of hydroxymethylation levels or other values, data are compressed. Compression typically is by Principal Component Analysis (PCA) or a similar technique for visualizing the structure of high-dimensional data. PCA is used to reduce dimensionality of the data (e.g., measured expression values) into uncorrelated principal components (PCs) that explain or represent a majority of the variance in the data, such as about 50, 60, 70, 75, 80, 85, 90, 95 or 99% of the variance. PCA allows the visualization of biomarker levels and the comparison of hydroxymethylation profiles, such as between normal or reference samples and test samples. PCA mapping, e.g., 3-component PCA mapping is used to map data to a three-dimensional space for visualization, such as by assigning first, second, and third PCs to the x-, y-, and z-axes, respectively.

In some embodiments, there is a linear correlation between hydroxymethylation levels of two or more biomarkers. Pearson's Correlation (PC) coefficients may be used to assess linear relationships (correlations) between pairs of values, such as between hydroxymethylation levels of a biomarker. This analysis may be used to linearly separate distribution in expression patterns, by calculating PC coefficients for individual pairs of the biomarkers (plotted on x- and y-axes of individual Similarity Matrices). Thresholds may be set for varying degrees of linear correlation, such as a threshold for highly linear correlation of (R.sup.2>0.50, or 0.40). Linear classifiers can be applied to the datasets. In one example, the correlation coefficient is 1.0.

In some embodiments, Feature Selection (FS) is applied to remove the most redundant features from a dataset, such as a hydroxymethylation biomarker dataset. FS enhances the generalization capability, accelerates the learning process, and improves model interpretability. In one aspect, FS is employed using a “greedy forward” selection approach, selecting the most relevant subset of features for the robust learning models. (Peng H, Long F, Ding C, “Feature selection based on mutual information: criteria of max-dependency, max-relevance, and min-redundancy,” IEEE Transactions on Pattern Analysis and Machine Intelligence, 2005; 27(8):1226-38). In some embodiments, SVM algorithms are used for classification of data by increasing the margin between the n data sets (Cristianini N, Shawe-Taylor J. An Introduction to Support Vector Machines and other kernel-based learning methods. Cambridge: Cambridge University Press, 2000).

Analytic classification of the hydroxymethylation biomarkers herein can be made according to predictive modeling methods that set a threshold for determining the probability that a sample (e.g., a cfDNA sample obtained from a patient) belongs to a given class (e.g., elevated risk of developing PDAC). The probability preferably is at least 50%, or at least 60%, or at least 70%, or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUROC (area under the ROC curve) or accuracy, of a particular value, or range of values. Area under the curve measures are useful for comparing the accuracy of a classifier across the complete data range. Classifiers with a greater AUC have a greater capacity to classify unknowns correctly between two groups of interest. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC of at least about 0.7, at least about 0.75, at least about 0.8, at least about 0.85, at least about 0.9, or higher.

As is known in the art, the relative sensitivity and specificity of a predictive model can be adjusted to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about 0.7, at least about 0.75, at least about 0.8, at least about 0.85, at least about 0.9, at least about 0.95, at least about 0.98, at least about 0.99, or higher.

Raw data can be initially analyzed by measuring the hydroxymethylation level for each biomarker. The data can be manipulated, for example, raw data can be transformed using standard curves, and the average of multiple measurements, if made, can be used to calculate the average and standard deviation for each patient. The data are then input into a selected predictive model, which will classify the sample. The resulting information can be communicated to a patient or health care provider, usually in the form of a written report.

To generate a predictive model for PDAC, a robust data set, comprising known control samples and samples corresponding to PDAC, is used in a training set. A sample size can be selected using generally accepted criteria. As discussed above, different statistical methods can be used to obtain a highly accurate predictive model. The examples herein provide representative such analyses.

In one embodiment, hierarchical clustering is performed in the derivation of a predictive model, where the Pearson correlation is employed as the clustering metric. One approach is to consider a dataset as a “learning sample” in a problem of “supervised learning.” CART is a standard in applications to medicine (Singer, Recursive Partitioning in the Health Sciences (Springer, 1999)) and can be modified by transforming any qualitative features to quantitative features, sorting them by attained significance levels, and a selected regularization method then applied (e.g., elastic net or lasso).

In some embodiments, the predictive models include Decision Tree, which maps observations about an item to a conclusion about its target value (Zhang et al., “Recursive Partitioning in the Health Sciences,” in Statistics for Biology and Health (Springer, 1999.). The leaves of the tree represent classifications and branches represent conjunctions of features that devolve into the individual classifications.

The predictive models and algorithms may further include Perceptron, a linear classifier that forms a feed forward neural network and maps an input variable to a binary classifier (Gallant (1990), “Perceptron-based learning algorithms,” in IEEE Transactions on Neural Networks 1(2):179-191). In this model, the learning rate is a constant that regulates the speed of learning. A lower learning rate improves the classification model, while increasing the time to process the variable (Markey et al. (2002) Comput Biol Med 32(2):99-109).

As explained earlier herein, the invention provides a method for determining the likelihood that an individual has or will develop PDAC, including a method for determining the likelihood that an identified pancreatic lesion is cancerous. Also provided are diagnostic, prognostic, and predictive uses of hydroxymethylation profiles, as well as uses in patient monitoring, evaluation of treatment options, and evaluation of treatment efficacy, wherein, in each method of use, the hydroxymethylation profile generated is combined with clinical parameters and optionally with one or more other risk factors in each method of use. All of the methods involve the generation of a hydroxymethylation profile comprising measurements of hydroxymethylation levels at each of a plurality of hydroxymethylation biomarker loci.

Among the provided diagnostic, prognostic, and predictive methods are those which employ statistical analysis and biomathematical algorithms and predictive models to analyze the detected hydroxymethylation information. Some embodiments include methods and systems for analyzing the hydroxymethylation information in classification, staging, prognosis, treatment design, evaluation of treatment options, prediction of outcomes (e.g., predicting development of metastases), and the like.

Also provided are methods that use evaluation of hydroxymethylation levels at the biomarker loci in treatment development and patient monitoring, including evaluation of a patient's response to treatment and patient-specific or individualized treatment strategies. In some embodiments, the methods are used in conjunction with treatment, for example, by generating a hydroxymethylation profile weekly or monthly before and/or after treatment. As the hydroxymethylation levels at certain biomarker loci correlate with the progression of disease, ineffectiveness or effectiveness of treatment, and/or the recurrence or lack thereof of disease, the regular generation of hydroxymethylation profiles within an extended monitoring or treatment period is useful. In some aspects, the information obtained may indicate that a different treatment strategy is preferable. Thus, provided herein are therapeutic methods, in which biomarker evaluation is performed prior to treatment, and then used to monitor therapeutic effects.

More specifically, at various points in time after initiating or resuming treatment, significant changes in hydroxymethylation levels at one or more of the biomarker loci may be seen, indicating that a therapeutic strategy is or is not successful, that disease is recurring, or that a different therapeutic approach should be used. In some embodiments, the therapeutic strategy is changed following a hydroxymethylation analysis, such as by adding a different therapeutic intervention, either in addition to or in place of a prior approach, by increasing or decreasing the aggressiveness or frequency of the approach, or by stopping or reinstituting a treatment regimen.

In another aspect, the hydroxymethylation levels at each of the biomarker loci are used to identify the presence of PDAC or a risk of developing PDAC for the first time.

In some aspects, the methods determine whether or not the assayed patient is responsive to treatment, such as a subject who is clinically categorized as in complete remission or exhibiting stable disease. In some aspects, methods are provided for distinguishing treatment-responsive and non-responsive patients, and for distinguishing patients with stable disease or those in complete remission, and those with progressive disease.

In various aspects, the methods and systems make such calls with at least at or about 65, 70, 75, 80, 81, 82, 83, 84, 85, 86, 87, 88, 89, 90, 91, 92, 93, 94, 95, 96, 97, 98, 99, or 100% correct call rate (i.e., accuracy), specificity, or sensitivity.

All of the aforementioned methods are encompassed by the present invention. Preferred methods herein include, without limitation:

a method for determining the likelihood that a patient has PDAC or will develop PDAC;

a method for evaluating the risk that an identified pancreatic lesion in a patient is cancerous;

a method for monitoring an identified pancreatic lesion in a patient, which involves an analysis of hydroxymethylation changes over time;

a method for managing a patient with an identified pancreatic lesion, which involves an evaluation of treatment options based on a hydroxymethylation profile;

a method for monitoring the effectiveness of treatment in a patient with an identified pancreatic lesion, which involves an analysis of hydroxymethylation profiles generated at selected time intervals within an extended monitoring period; and

a method for reducing unnecessary surgical resection of a pancreatic lesion by evaluating the risk that the pancreatic lesion is cancerous using a hydroxymethylation profile.

### EXAMPLE 1

A. Study Design and Methods:

(i) Clinical Cohorts and Study Design:

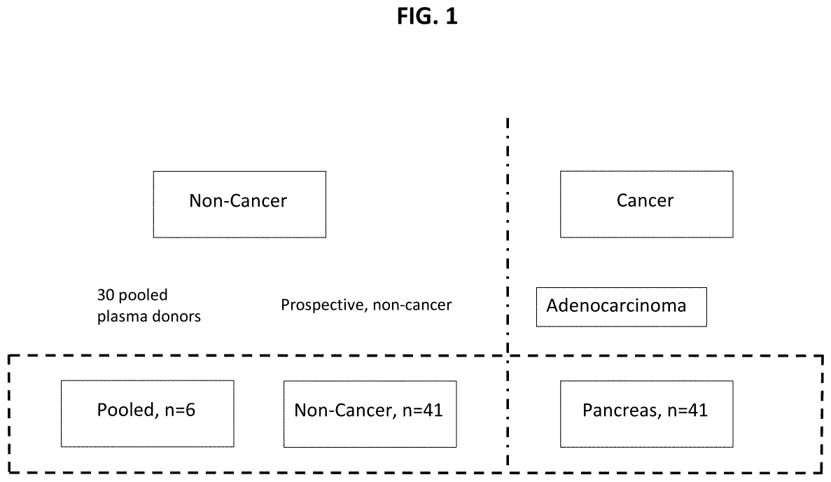

A case-control study was performed using plasma obtained from 92 subjects with or without pancreatic ductal adenocarcinoma, 41 in a non-cancer cohort and 51 in a cancer cohort, each of whom provided informed consent and contributed biospecimens in studies approved by the Institutional Review Boards (IRBs) at participating sites in the United States and Germany. Plasma samples for the non-cancer cohort were obtained from subjects enrolled prospectively at five sites in the United States, following review and approval of the study protocol by each site's participating investigator(s). Criteria for subject eligibility for inclusion in the analysis included age greater than or equal to 18 years for all subjects and no treatment with medication for any disease at the time of blood collection. FIG. 1 schematically depicts the study cohorts.

Cancer cohort: Plasma samples for the cancer cohort were obtained from subjects who had undergone management for PDAC in the United States or Germany. Criteria for subject eligibility: 1) no cancer treatment, e.g., surgical, chemotherapy, immunotherapy, targeted therapy, or radiation therapy, prior to study enrollment and blood specimen acquisition; and 2) a confirmed pathologic diagnosis of adenocarcinoma inclusive of all subtypes.

Non-cancer cohort: Subject exclusion criteria for the non-cancer cohort also included any of the following: a cancer diagnosis within prior six months; surgery or invasive procedure requiring general anesthesia within prior month; non-cancer systemic therapy associated with molecularly targeted immune modulation; concurrent or prior pregnancy within previous 12 months; history of organ tissue transplantation; history of blood product transfusion within one month; and major trauma within six months. Clinical data required for all subjects included age, gender, smoking history, and both tissue pathology and grade, and were managed in accordance with the guidance established by the Health Insurance Portability and Accountability Act (HIPAA) of 1996 to ensure subject privacy. The clinical characteristics of both cohorts are set forth in Table 6:

TABLE 6

| | No Cancer | Cancer |
|---|---|---|
| Age⁺ | | |
| | 66.0 | 71.2 |
| Gender (%) | | |
| Male | 60.0 | 45.1 |
| Smoking History | | |
| Status (%) | | |
| Current | 19.5 | 19.6 |
| Former | 29.3 | 37.3 |
| None | 51.2 | 43.1 |
| Pack-Years⁺ | | |
| Current | 5.3 | 29.6 |
| Former | 20.5 | 24.2 |
| None | NA | NA |
| Pack-Years⁺ | | |
| All | 14.4 | 25.7 |
| Time Since Cessation⁺ | | |
| Months | 264.2 | 272.3 |
| Stage | | |
| I | NA | 18 |
| II | NA | 61 |
| III | NA | 7.8 |
| IV | NA | 14 |

⁺mean of Non-Cancer and Cancer
Other values are percentages of each category in the Non-Cancer and Cancer groups.

As may be seen in the table, there were no statistically significant differences in subject age or gender between the two cohorts, but there was a statistically significant greater tobacco exposure in the PDAC cohort, as would be expected given that smoking is a common risk factor in pancreatic cancer.

(ii) Plasma collection: Plasma was isolated from whole blood specimens obtained by routine venous phlebotomy at the time of subject enrollment. For cancer subjects, whole blood was collected in K3EDTA tubes (Sarstedt, Nümbrecht, Germany) with isolation of plasma within 4 h of phlebotomy by centrifugation at 1,500 g for 10 min at RT, followed by transfer of the plasma layer to a new tube for centrifugation at 3,000 g for 10 min at RT, with plasma aliquots used for isolation of cell-free DNA (cfDNA) or stored at −80° C. For non-cancer subjects, whole blood was collected in Cell-Free DNA BCT® tubes according to the manufacturer's protocol (Streck, La Vista, Nebr.) (https://www.streck.com/collection/cell-free-dna-bct/). Plasma was aliquoted for subsequent cfDNA isolation or storage at −80° C.

(iii) Cell-free DNA isolation: Cell-free DNA was isolated using the QIAamp Circulating Nucleic Acid Kit (QIAGEN, Germantown, Md.) following the manufacturer's protocol excepting the omission of carrier RNA during cfDNA extraction. Two milliliter plasma volumes (cancer) or four milliliter plasma volumes (non-cancer) were lysed for 30 minutes prior to collection of nucleic acids; all cfDNA eluates were collected in a volume of 60 μl buffer. All cfDNA eluates were quantified by Bioanalyzer dsDNA High Sensitivity assay (Agilent Technologies Inc, Santa Clara, Calif.) and Qubit dsDNA High Sensitivity Assay (Thermo Fisher Scientific, Waltham, Mass.) to ensure the absence of contaminating high molecular weight DNA emanating from white blood lysis.

(iv) 5-Hydroxymethylcytosine (5hmC) enrichment assay: 5hmC-enriched libraries were prepared using the cell-free “5hmC-Seal” method described in International Patent Publication WO 2017/176630 to Quake et al., Song et al. (2011) 29: 68-72, and Han et al. (2016) Mol. Cell 63:711-19, the disclosures of which are incorporated by reference herein. Briefly, hMe-Seal is a low-input, whole-genome cell-free 5hmC sequencing method based on selective chemical labeling, in which β-glucosyltransferase is used to selectively label 5hmC with a biotin moiety via an azide-modified glucose for pull-down of 5hmC-containing DNA fragments for sequencing. In implementing hMe-Seal in the present case, the cfDNA was normalized to 10 ng total input for each assay and ligated with sequencing adapters, followed by selective labeling of 5hmC with β-GT, and affinity enrichment via selective pull-down of DNA fragments containing biotin-labeled 5hmC by binding to Dynabeads M270 Streptavidin (Thermo Fisher Scientific, Waltham Mass.). PCR was then carried out directly from the beads (i.e., instead of eluting the captured DNA) to minimize sample loss during purification. All libraries were quantified by Bioanalyzer dsDNA High Sensitivity assay (Agilent Technologies Inc, Santa Clara, Calif.) and Qubit dsDNA High Sensitivity Assay (Thermo Fisher Scientific, Waltham, Mass.) and normalized in preparation for sequencing.

(v) DNA sequencing and alignment: DNA sequencing was performed according to manufacturer's recommendations with 75 base-pair, paired-end sequencing using a NextSeq550 instrument with version 2 reagent chemistry (Illumina, San Diego, Calif.). Sixteen libraries were sequenced per flowcell. Raw data processing and demultiplexing was performed using the Illumina BaseSpace Sequence Hub to generate sample-specific FASTQ output. Sequencing reads were aligned to the hg19 reference genome using BWA-MEM with default parameters (Li et al. (2010) Bioinformatics 26: 589-595).

(vi) Peak detection: BWA-MEM read alignments were employed to identified regions or peaks of dense read accumulation that mark the location of a hydroxymethylated cytosine residue in a CpG context. Prior to identifying peaks BAM files containing the locations of aligned reads were filtered for poorly mapped (MAPQ <30) and not properly paired reads. 5hmC peak calling was carried out using MACS2 (https://github.com/taoliu/MACS) with a p-value cut off=1.00e-5. Identified 5hmC peaks residing in “blacklist regions” as defined elsewhere (https://sites.google.com/site/anshulkundaje/projects/blacklists) and read date on chromosomes X, Y and mitochondrial genome were also removed. Computation of genomic feature enrichment overlap 5hmC peaks were performed using the HOMER software (http://homer.ucsd.edu/homer/) with default parameters.

(vii) Chromatin modifications: Chromatin modifications (H3K4me1, H3K4me3 and H3K27ac) were identified in histone maps of the pancreatic cancer cell line PANC-1 and downloaded from ENCODE ChIP-Seq repository (https://genome.ucsc.edu/encode/dataMatrix/encodeChipMatrixHuman.html). Determinations of enrichment were calculated via Odds ratio using the Fisher Exact Test via the program bedtools fisher. For comparisons between PDAC and non-cancer, the Wilcoxon test was used, and for across stages comparison, the Kruskal-Wallis Test was employed.

(viii) Differential representation analysis: For the purpose of reliably identifying gene bodies with differential representation between the PDAC and the non-cancer groups, we closely followed the RNA-Seq workflow outlined in Law et al. (2016) F1000Research 5:1408, including much of the preliminary QC steps. The following workflow was adopted for data pre-processing: (i) transforming the data from raw counts to log₂ (counts per million), (ii) removing genes that were found to be weakly represented, (iii) normalizing the gene representation distributions, and (iv) performing unsupervised clustering of samples. To accomplish differential representation analysis, we applied the following steps: (i) creating a design matrix and PDAC vs non-cancer cohorts, (ii) removing heteroscedasticity from the data, (iii) fitting the regression models for the comparison of interest, PDAC vs non-cancer, and (iv) examining the number of differentially represented genes. Default settings were used when appropriate. To remove weakly represented genes, genes were excluded which did not have greater than 3 counts per million reads in at least 20 samples. This filter excludes roughly 12% of the genes. For the identification of the significantly differentially represented regions, the method of Benjamini and Hochberg (Benjamini et al. (1995) Journal of the Royal Statistical Society, Series B (Methodological) 289-300) was used to obtain p-values adjusted for multiple comparisons. Adjusted p-value and false discovery rate (FDR) were used interchangeably.

(ix) Predictive Modeling for Detection of PDAC in cfDNA:

For the purpose of assessing the feasibility of building classifiers that can discriminate between PDAC and non-cancer samples based on the 5hmC representation of gene bodies, we evaluated the performance of two forms of regularized logistic regression models, namely the lasso and the elastic net, which are commonly used in the classification context, where the number of examples are few and the number of features is large. See Friedman et al. (2010) Journal of Statistical Software 33: 1-22 for a description of the general elastic net procedure. Software implementation of these methods can be found at https://cran.r-project.org/web/packages/glmnet/index.html. Weakly represented genes were excluded from analysis as described in the preceding section.

All training and fitting was done on 75% of the samples selected at random in a balanced way to keep the ratio of the number of PDAC to non-cancer samples similar in both the training and testing subsets. Before any fitting, genes were filtered to include the 65% of the most variable genes for model fitting task. The filter was designed using training samples only and was done in a way to ensure that genes of all levels of 5hmC representation were included.

Both assessed regularization methods, the lasso and the elastic net, require specifying hyperparameters which control the level of regularization used in the fit. Hyperparameters of the regularization model were selected based on out-of-fold performance on 30 repetitions of 5-fold cross-validated analysis of the training data. Out-of-fold assessments were based on the samples in the left-out fold at each step of the cross-validated analysis.

The hyperparameter values that lead to the best out-of-fold performance were then used to fit the final models, which were fitted to the entire set of samples including both training and testing subsets, to provide a sense of the generalizability of the performance observed in the local training and testing data sets.

To evaluate the effect of feature selection on prediction performance, we repeated the training and evaluation task based on a filtered set of genes that included genes found to be significantly differentially represented, having a 1.5 fold differential 5hmC representation, and a level of representation exceeding the median level (log₂ CPM ≥4). This filter was designed based on training data statistics only.

B. Results:

(i) Sequencing Results and Metrics:

(ii) Cohort-Based Distributions of 5hmC Densities into Functional Regions:

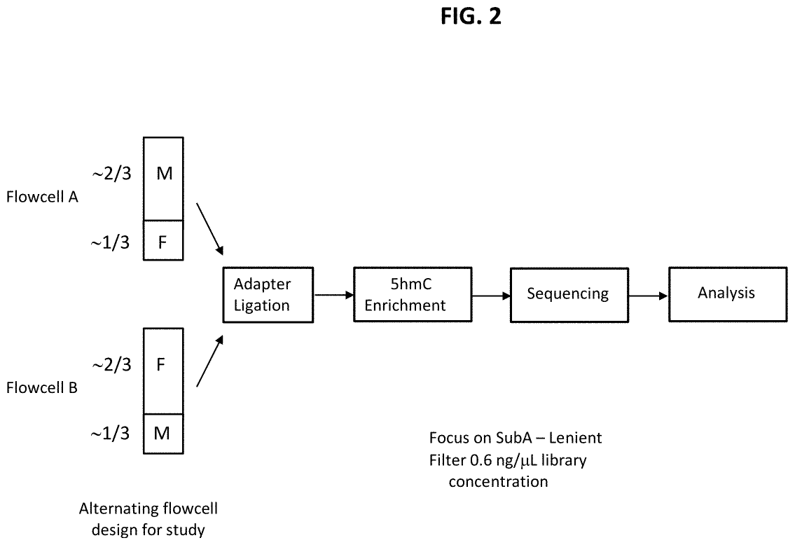

Filtering criteria to enable the determination of high quality 5hmC libraries were established yielding 51 PDAC and 41 non-cancer subjects. These criteria were established from previous studies (Song et al. (2017) and extensive analysis did not reveal batch processing effects occurring specifically in either study cohort. PDAC samples that were exclusively male or female were combined with non-cancer samples that were exclusively either female or male in a ratio of 2:1, respectively, following a block randomization scheme. This generated two batch types (FIG. 2) and enabled the identification of sample swaps, none of which were detected. A single pooled non-cancer sample (30 non-cancer donors were combined into one plasma pool from which cfDNA was isolated) served as a technical/process control for each of the batches in the study 5hmC enrichment libraries were sequenced to produce a median number of unique read pairs of 9.1 and 10.7 million in the PDAC and non-cancer cohorts respectively.

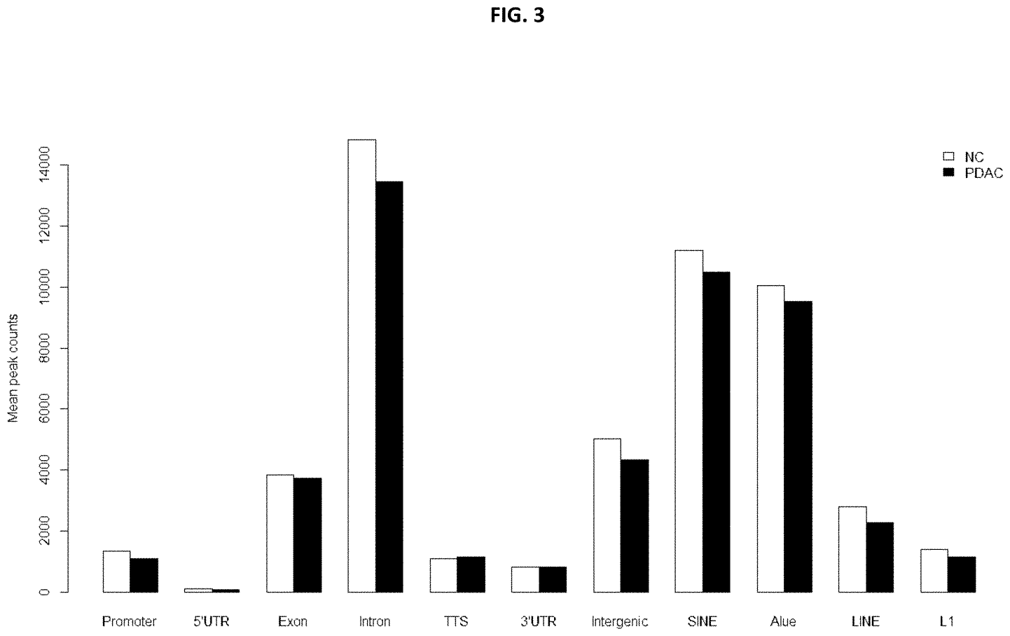

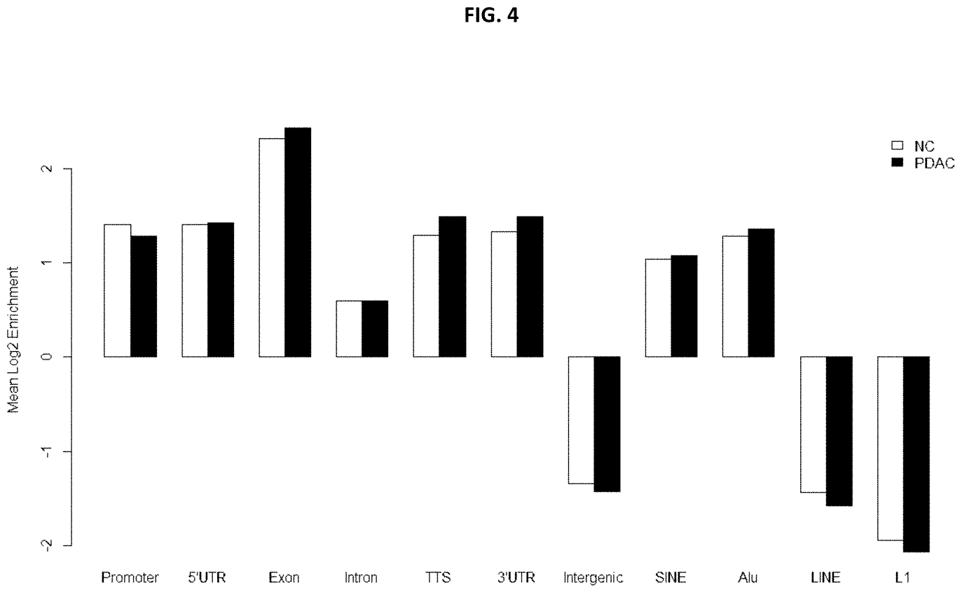

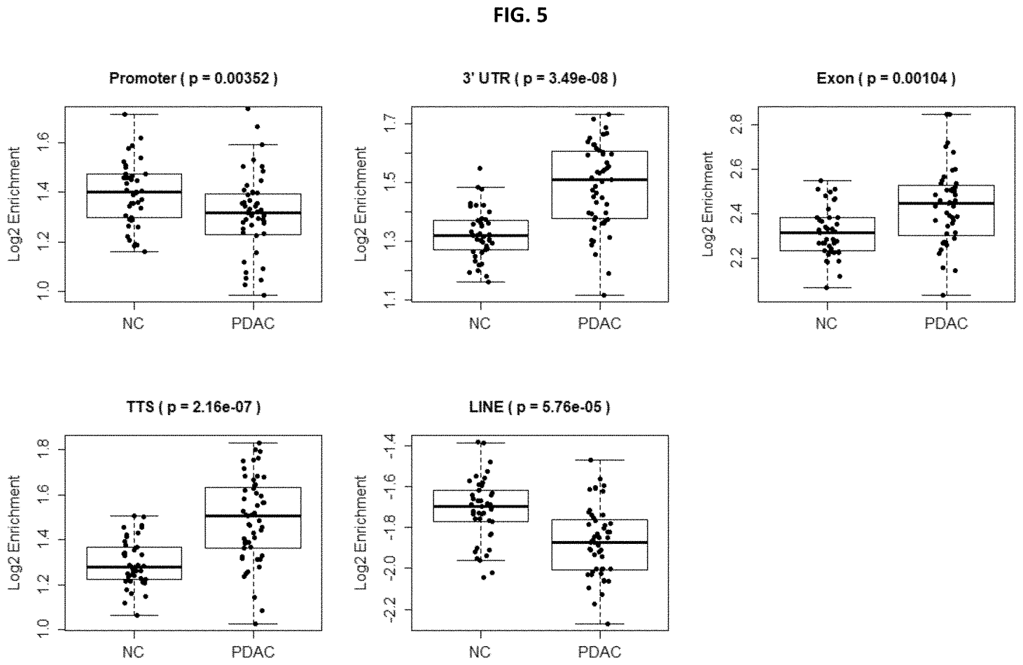

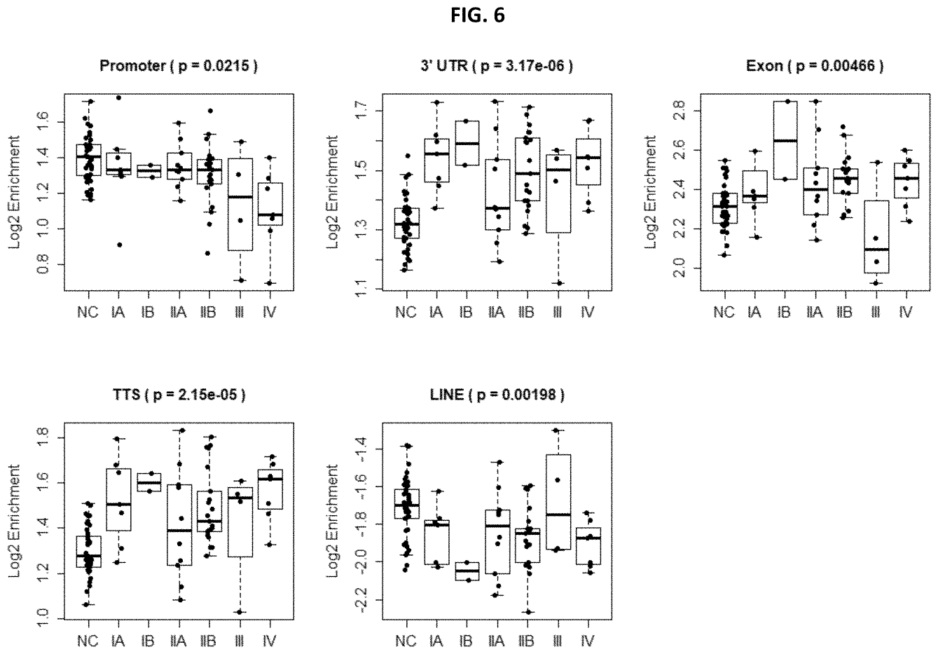

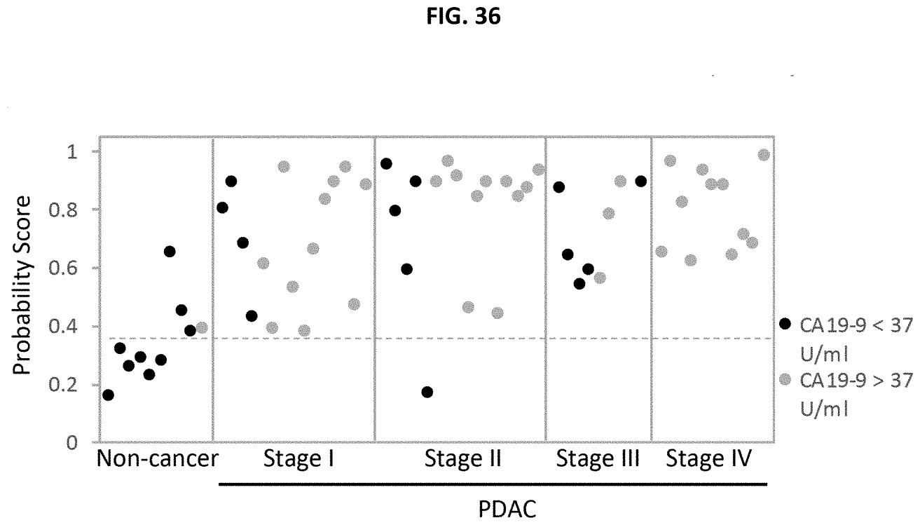

The vast majority of 5hmC loci, as measured by increased read density and detected by MACS2 as peaks, were found to occur, on average, in non-coding, intragenic regions of the genome, i.e., intronic, transposon repeats—SINEs and LINEs, and intergenic, as illustrated in FIG. 3, with no preferential 5hmC distribution in any one disease cohort (also see FIG. 39). These regions displayed low 5hmC enrichment (intron, FIG. 4) or even depletion of 5hmC sites (intergenic and LINE elements, FIG. 4). Instead, 5hmC enrichment occurred more frequently in promoters, UTRs, exons, TTS regions, and SINE elements as measured relative to the genome background. Significant differences in the enrichment of 5hmC peaks in functional regions were observed in a disease cohort specific manner. Increases in enrichment in PDAC were measured in exons, 3′UTR, and TTS, whereas decreases were found in promoter and LINEs, which themselves were either 5hmC-enriched or 5hmC-depleted, respectively (FIG. 5). These global changes were found to occur in a statistically significant manner in each cohort and were also found to occur in a cancer stage specific manner, with gradual increases (exon, 3′UTR and TTS) or decreases (promoter and LINE) in later stage patients (FIG. 6).

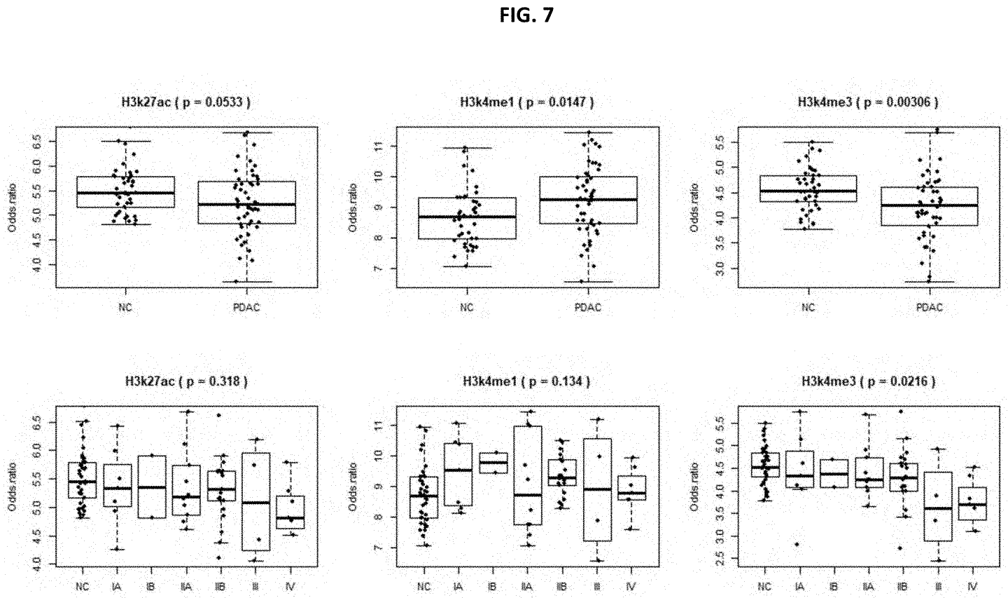

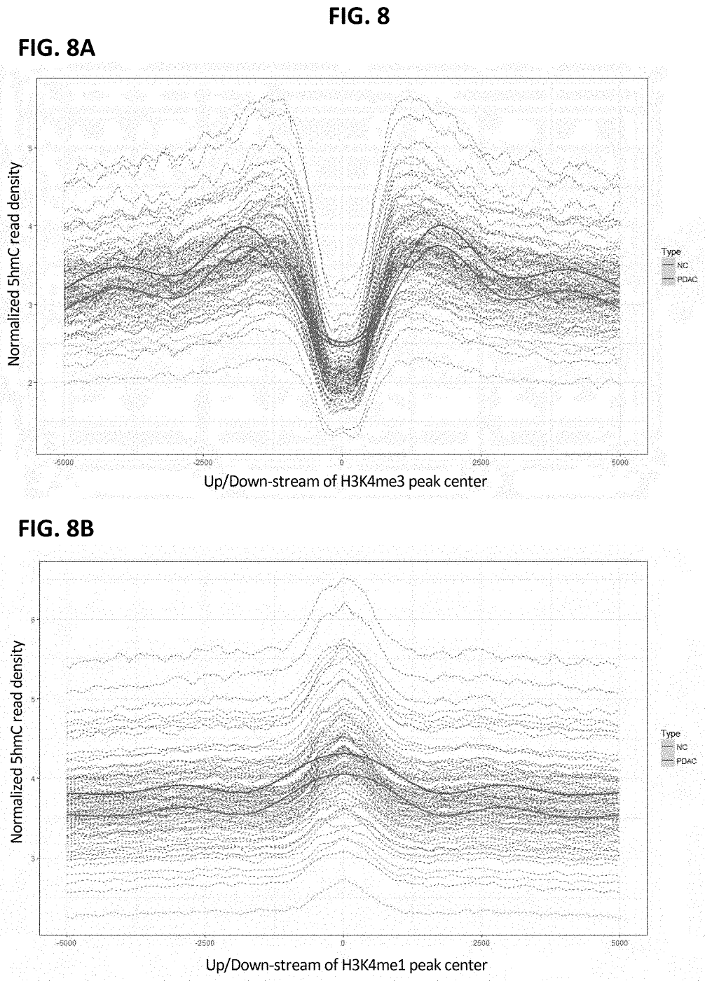

Investigation of 5hmC occupancy and its associated changes in PDAC, with respect to chromatin state: Post-translational chemical modifications like methylation and acetylation on histone proteins were inferred in relation to 5hmC occupancy, using the existing histone maps from PANC-1 cell lines (see LeRoy et al. (2013) Epigenetics & Chromatin 6:20), for which epigenetic data were made available by ENCODE The H3K27Ac mark had the largest density of 5hmC occupancy in both the cancer and non-cancer cohort and the highest similarity to the Panc1 cell line histone map (FIG. 8A). Conversely, H3K27Me3 exhibited the lowest density of 5hmC occupancy in both cohorts and the lowest similarity to the PANC-1 cell line histone map (FIG. 8B). Notably, reduced overlap with 5hmC was observed in PDAC coincident with loci associated with H3K4me3 and H3K27ac, both of which mark transcriptionally active states (FIG. 7). There was no significant global difference between PDAC and non-cancer cohorts in 5hmC peak overlap with H3K4me1-associated loci, which mark enhancer regions (FIG. 7). Furthermore, 5hmC peak overlap with H3K4me3-associated loci was significantly reduced with progressive disease as observed when the PDAC cohort was subdivided into disease stages (p=0.0003—FIG. 7, bottom right panel). The H3K27Ac mark had the largest density of 5hmC occupancy in both the cancer and non-cancer cohort and the highest similarity to the Panc1 cell line histone map (FIG. 8A). Conversely, H3K27Me3 exhibited the lowest density of 5hmC occupancy in both cohorts and the lowest similarity to the PANC-1 cell line histone map (FIG. 8B). This was consistent with increased expression of the actively transcribed genes in PANC-1 cancer cell line, in the PDAC cohort compared to non-cancer cohort. Conversely, there were no statistically significant changes detected globally in 5hmC peak occupancy over H3K4me1- and H3K27ac-associated loci with progressive disease (FIG. 7, bottom panels). Amongst the three histone maps from PANC-1 cell lines, H3K4me1 has the most similarity with 5hmC occupancy in both the PDAC and non-cancer cohort whereas H3K4me3 histone map has the lowest similarity, as calculated by Jaccard index. Moreover, 5hmC read density was highest at the peak center for H3K4me1-associated sites in general whereas it was lowest at the peak center for H3K4me3-associated sites (FIGS. 8A and 8B). Overall, 5hmC was found to exhibit diverse patterns over these different chromatin contexts, which is potentially indicative of varied interplay between hydroxymethylation and different histone modifications.

(iii) Identification of Disease Specific Genes from Plasma Samples:

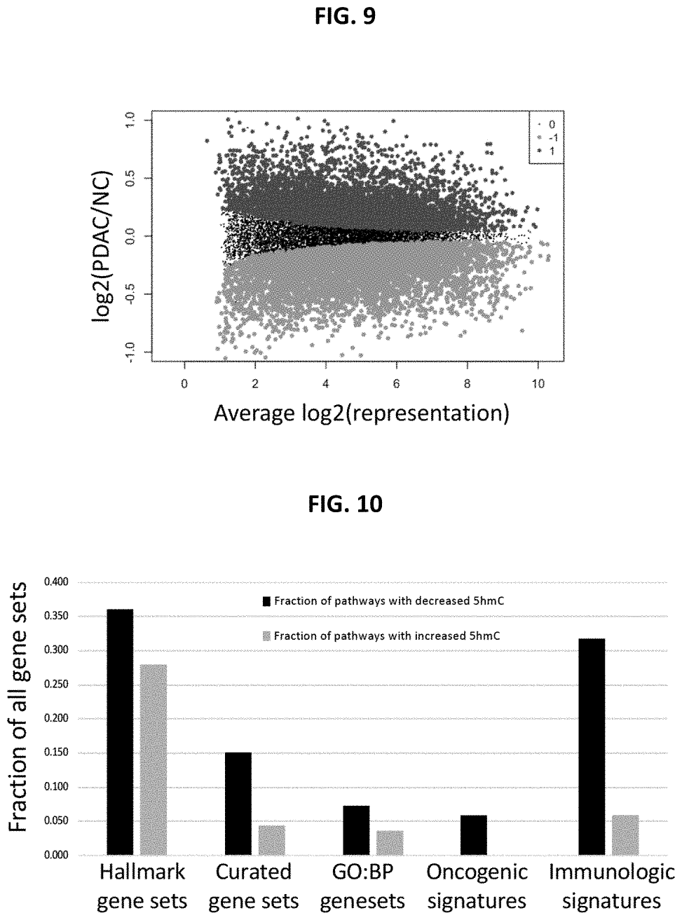

Differential analysis of 5hmC densities in genes revealed 6,496 and 6,684 genes with an increased and decreased 5hmC density, respectively, in PDAC, compared to non-cancer samples (FIG. 9, adjusted p-value<0.05). Further filtering of this gene set (fold change ≥1.5 in PDAC versus non-cancer and average log 2 CPM ≥4 counts, 142 genes total) revealed annotated genes with increased 5hmC density and whose biology is related to pancreas development (GATA4, GATA6, PROX1, ONECUT1) and/or implicated in cancer (YAP1, TEAD1, PROX1, ONECUT2/ONECUT1, IGF1 and IGF2). Inspection of the Molecular Signatures Database (MSigDB) for relevant pathways comprising the 142 genes with enriched 5hmC densities revealed a numeric preponderance of pathways down-regulated in liver cancer (5 of the top 10 most significant pathways, as indicated in Table 6). The differential representation analysis coupled with filtering (fold change ≥|1.5| in PDAC versus non-cancer and log CPM of 5hmC ≥4) also revealed 178 genes with a decreased 5hmC density in pancreatic cancer cfDNA (Table 8). Closer inspection of these pathways with decreased 5hmc representation revealed fundamental pathways in immune system regulation (3 of the top 10 most significant pathways, as may be seen in Table 7:

TABLE 7
Top ten pathways represented by 142 genes with increased 5hmC
density in PDAC samples versus non-cancer samples
(also see Collin et al. (2018), “Detection of Early Stage Pancreatic Cancer Using
5-Hydroxymethylcytosine Signatures in Circulating Cell-Free DNA,”
bioRxiv, doi:https://dx.doi.org/10.1101/422675, incorporated by reference herein):

| Gene | | | | FDR |
|---|---|---|---|---|
| Set* | Description | k/K | p-value | q-value |
| 1 | Genes down-regulated in liver tissue upon knockout | 0.0892 | 3.92E−17 | 5.61E−13 |
| | of HNF1A (GeneID = 6927). | | | |
| 2 | Genes down-regulated in tumor compared to non- | 0.2041 | 2.35E−16 | 1.68E−12 |
| | tumor liver samples from patients with hepatocellular | | | |
| | carcinoma (HCC). | | | |
| 3 | The chemical reactions and pathways involving small | 0.0175 | 5.53E−16 | 2.64E−12 |
| | molecules (any low molecular weight, monomeric, | | | |
| | non-encoded molecule). | | | |
| 4 | Liver-selective genes. | 0.0615 | 7.06E−16 | 2.72E−12 |
| 5 | Genes from subtype S3 signature of HCC: hepatocyte | 0.0564 | 2.73E−15 | 7.81E−12 |
| | differentiation. | | | |
| 6 | Genes down-regulated in liver tumor compared to the | 0.0547 | 4.22E−15 | 1.01E−11 |
| | normal adjacent tissue. | | | |
| 7 | The chemical reactions and pathways involving lipids | 0.0216 | 5.47E−15 | 1.12E−11 |
| | and lipidic molecules (fatty alcohols, sphingoids, | | | |
| | phospholipids, glycolipids, sterols, etc.). | | | |
| 8 | The chemical reactions and pathways involving | 0.0241 | 7.39E−15 | 1.32E−11 |
| | organic acids. | | | |
| 9 | Any process that results in a change in state or activity | 0.0179 | 1.08E−13 | 1.61E−10 |
| | of a cell or an organism (movement, secretion, enzyme | | | |
| | production, gene expression, etc.) as a result of a | | | |
| | stimulus arising within the organism. | | | |
| 10 | Down-regulated genes distinguishing between early | 0.0409 | 2.98E−13 | 4.27E−10 |
| | gastric cancer (EGC) and normal tissue samples. | | | |

```
*Gene set names:
  1: SERVITJA_LIVER_HNF1A_TARGETS_DN
  2: LEE_LIVER_CANCER
  3: GO_SMALL_MOLECULE_METABOLIC_PROCESS
  4: HSIAO_LIVER_SPECIFIC_GENES
  5: HOSHIDA_LIVER_CANCER_SUBCLASS S3
  6: ACEVEDO_LIVER_TUMOR_VS_NORMAL_ADJACENT
  7: GO_LIPID_METABOLIC_PROCESS
  8: GO_ORGANIC_ACID_METABOLIC_PROCESS
  9: GO_RESPONSE_TO_ENDOGENOUS_STIMULUS
10: VECCHI_GASTRIC_CANCER_EARLY_DN
```

TABLE 8
Top ten pathways represented by 178 genes with decreased 5hmC density
in PDAC samples versus non-cancer samples (also see Collin et al. (2018), supra):

| Gene | | | | FDR |
|---|---|---|---|---|
| Set* | Description | k/K | p-value | q-value |
| 1 | Genes involved in hemostasis | 0.0708 | 1.80E−33 | 2.58E−29 |
| 2 | Any process that modulates the frequency, rate, or | 0.0314 | 1.06E−29 | 7.56E−26 |
| | extent of an immune system process. | | | |
| 3 | Genes down-regulated in CD34+ (GeneID = 947) | 0.108 | 9.52E−28 | 4.54E−24 |
| | cells by intermediate activity levels of STAT5A | | | |
| | (GeneID = 6776); predominant long-term growth | | | |
| | and self-renewal phenotype. | | | |
| 4 | Any process involved in the development or | 0.0242 | 1.62E−27 | 5.79E−24 |
| | functioning of the immune system. | | | |
| 5 | Any process that modulates the levels of body fluids. | 0.0553 | 1.76E−25 | 5.05E−22 |
| 6 | A change in the morphology or behavior of a cell | 0.0511 | 2.19E−25 | 5.23E−22 |
| | resulting from exposure to an activating factor such | | | |
| | as a cellular or soluble ligand. | | | |
| 7 | Any process that activates or increases the frequency, | 0.0381 | 8.38E−25 | 1.71E−21 |
| | rate, or extent of an immune system process. | | | |
| 8 | Any process that modulates the frequency, rate, or | 0.0558 | 1.11E−24 | 1.98E−21 |
| | extent of cell activation. | | | |
| 9 | Genes involved in platelet activation, signaling, and | 0.0962 | 3.27E−23 | 5.19E−20 |
| | aggregation. | | | |
| 10 | Any process that modulates the frequency, rate, or | 0.0429 | 1.04E−21 | 1.49E−18 |
| | extent of attachment of a cell to another cell or to | | | |
| | the extracellular matrix. | | | |

```
*Gene set names:
  1: REACTOME_HEMOSTASIS
  2: GO_REGULATION_OF_IMMUNE_SYSTEM_PROCESS
  3: WIERENGA_STATSA_TARGETS_DN
  4: GO_IMUNE_SYSTEM_PROCESS
  5: GO_REGULATION_OF_BODY_FLUID_LEVELS
  6: GO_CELL_ACTIVATION
  7: GO_POSITIVE_REGULATION_OF_IMMUNE_SYSTEM_PROCESS
  8: GO_REGULATION_OF_CELL_ACTIVATION
  9: REACTOME_PLATELET_ACTIVATION_SIGNALING_AND_AGGREGATION
10: GO_REGULATION_OF_CELL_ADHESION
```

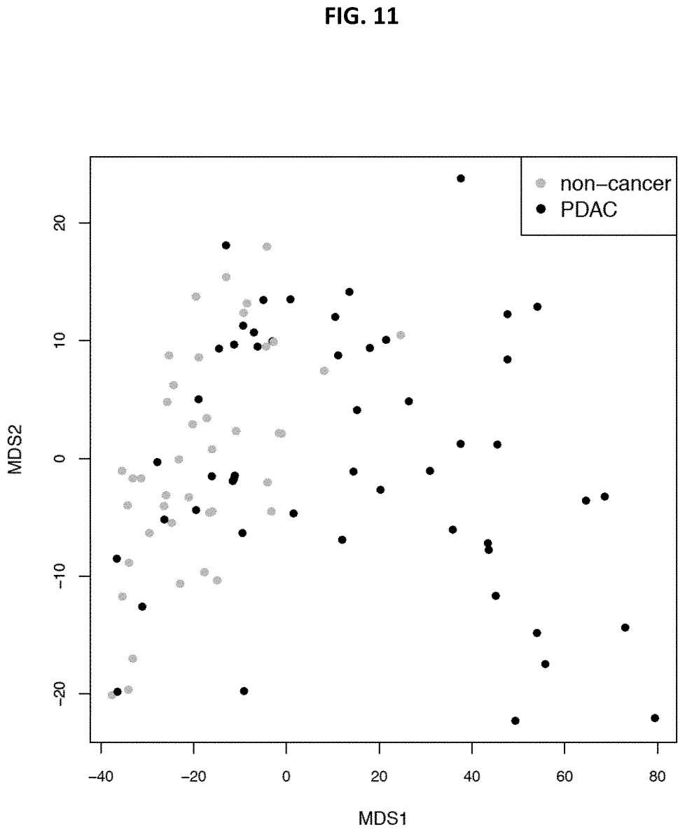

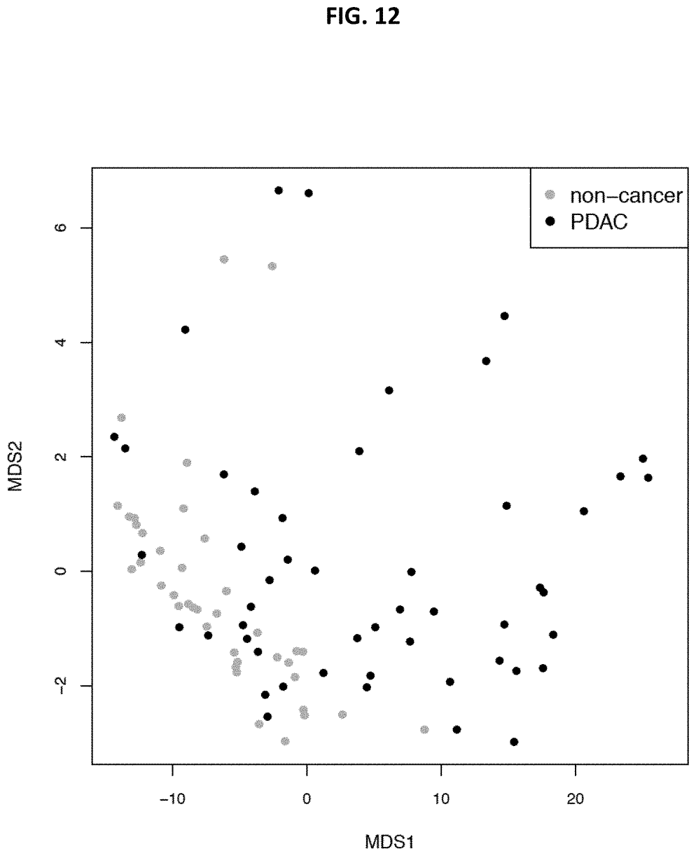

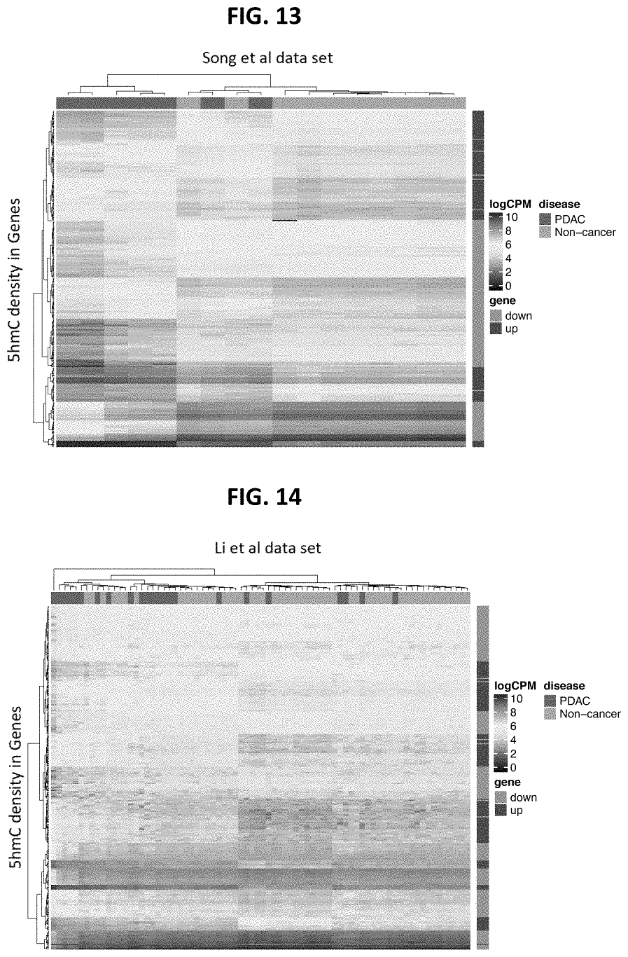

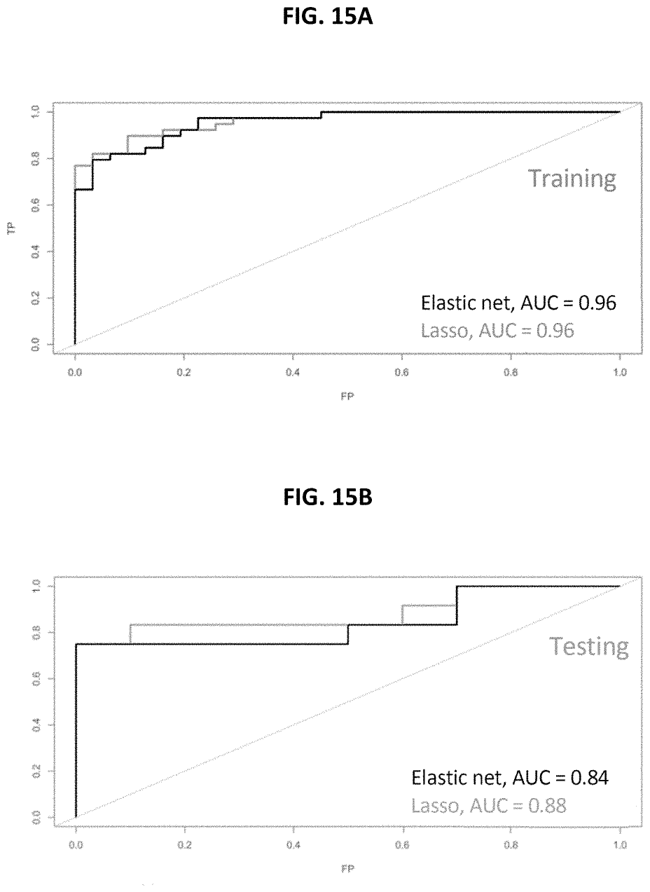

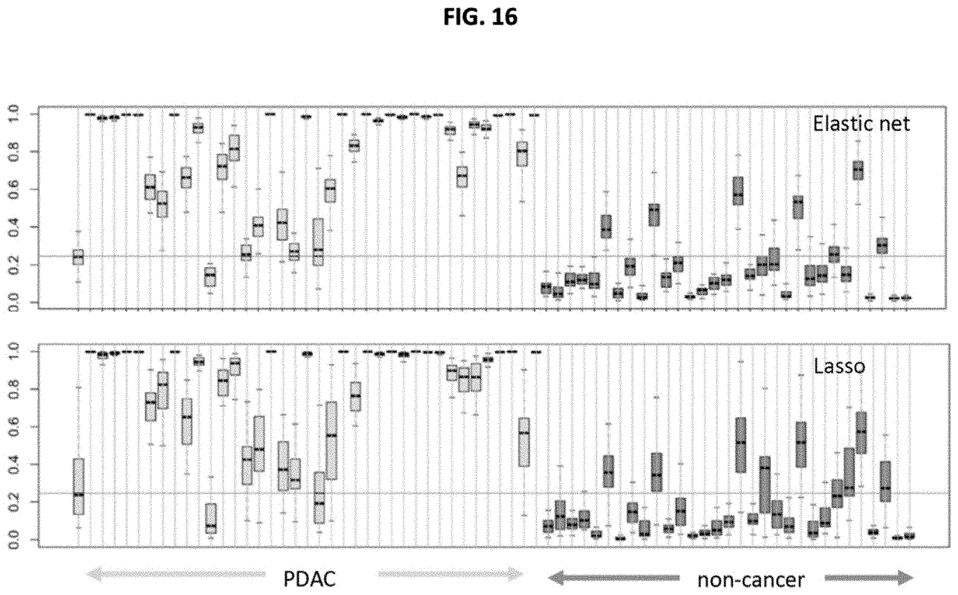

Expanding gene set enrichment analysis to include the full data set of all genes revealed that more than 30% of immune related pathways have reduced 5-hydroxymethylation across early and late stage PDAC (FIG. 10). Principal component analysis (PCA) using either the 13,180 genes with statistically significant variation in 5hmC counts (FIG. 11), or the 320 genes filtered at the extremes of 5hmC representation in PDAC (FIG. 12), revealed partitioning of the PDAC samples from the non-cancer samples equally well Furthermore, hierarchical clustering using the 320 genes revealed better partitioning of the 5hmC data from PDAC and non-cancer cfDNA from Song et al. (2017) (FIG. 13) compared with similar data from Li et al. (2017) (FIG. 14).

The results demonstrate the identification of a differentially represented gene set whose biological functions are congruent with both pancreatic development and cancer more broadly. Furthermore, 5-hydroxymethylation densities of these genes alone enable the partitioning of PDAC from non-cancer.

(iv) Explanation of Predictive Model Evaluation:

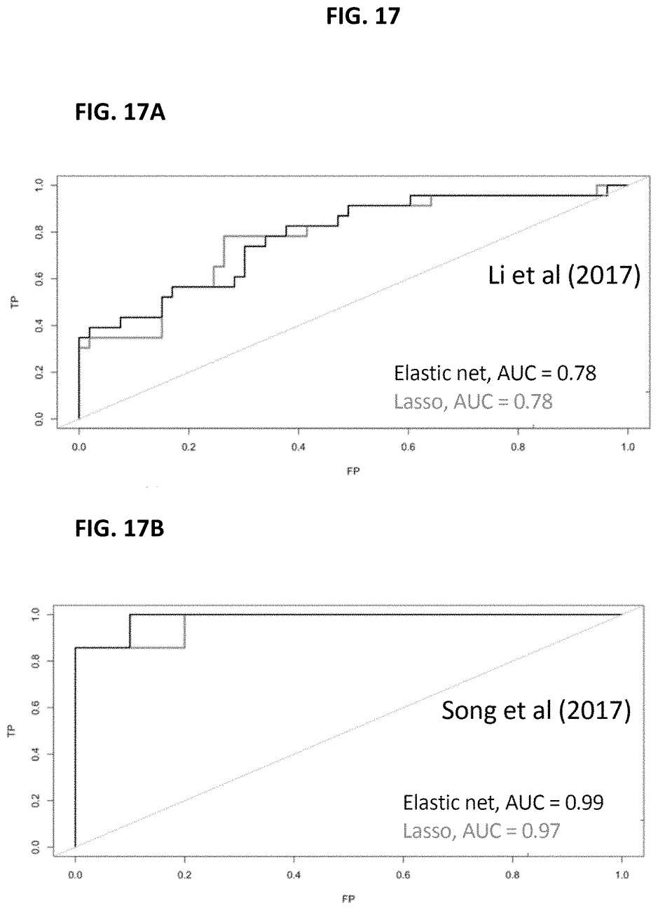

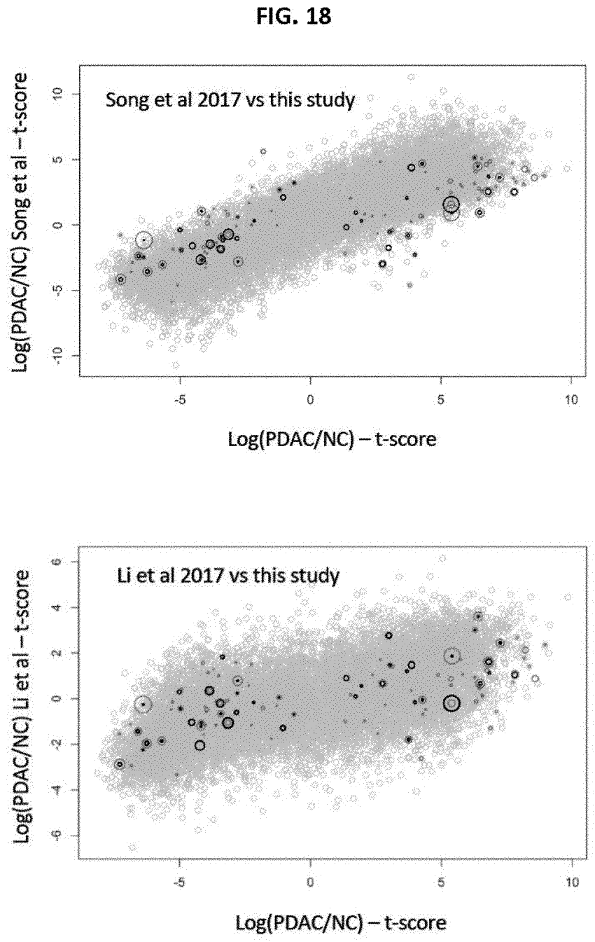

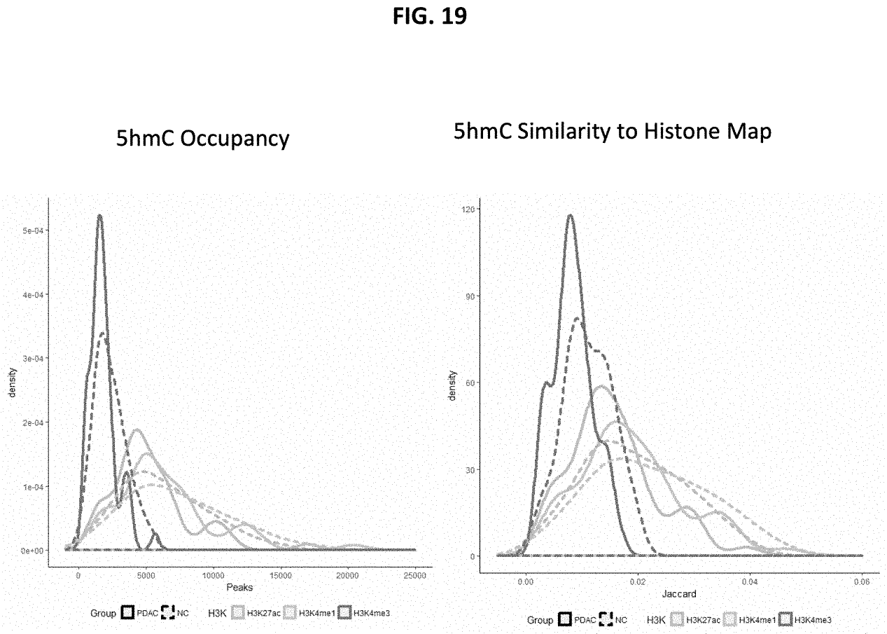

Both the elastic net (glmnet) and lasso (glmnet2) regularization methods require specifying hyperparameters that control the level of regularization used in the fit, selected as described in section (ix) of Part A. The out-of-fold assessments performed on the training data yielded an out-of-fold performance metric, Area Under Curve (AUC), of 0.96 (elastic net and lasso) with an internal sample test AUC of 0.84 (elastic net) and 0.88 (lasso) (FIGS. 15A and 15B). The distribution of probability scores shows that within training data, both models classify well (FIG. 16) but that marginally fewer misclassified samples were found with the elastic net model when the specificity was set at 75%, i.e., fewer cancer samples scored below the third quartile non-cancer score and fewer non-cancer samples scored above the same third quartile non-cancer score. The training model was then tested on an external validation set of patient samples. These include pancreatic cancer samples from Li et al. (2017) (pancreas subtype not specified; 23 subjects with pancreatic cancer, 53 healthy) and Song et al. (2017) (pancreas subtype specified as adenocarcinoma; 7 subjects with pancreatic cancer, 10 healthy). The validation set exhibited a performance with AUC=0.78 (elastic net and lasso) in the Li et al. (2017) data (FIG. 17A) and AUC=0.99 (elastic net) and 0.97 (lasso) in the Song et al. (2017) data (FIG. 17B).

The effect of feature selection on prediction performance was evaluated by filtering the initial set of significant genes (FIG. 9) to satisfy a 1.5-fold differential 5hmC representation in the PDAC cohort with median representation of gene counts of log₂ average 5hmC representation >4. The same regularized regression models were built using this set of 287 genes with increased 5hmC and 343 genes with decreased 5hmC counts, employing a similar setup for training and testing as defined previously (75% data used for training, 25% data used for test) and found training set AUC=0.96 (elastic net) and 0.94 (lasso). Not surprisingly, internal testing yields a high performance with AUC=0.92 (elastic net) and 0.93 (lasso). Hierarchical clustering of these significant genes (287 with increased 5hmC+343 with decreased 5hmC) showed good partitioning of the pancreatic cancer samples in the Stanford data set (FIG. 13) but less pronounced separation of the Chicago data (FIG. 14).

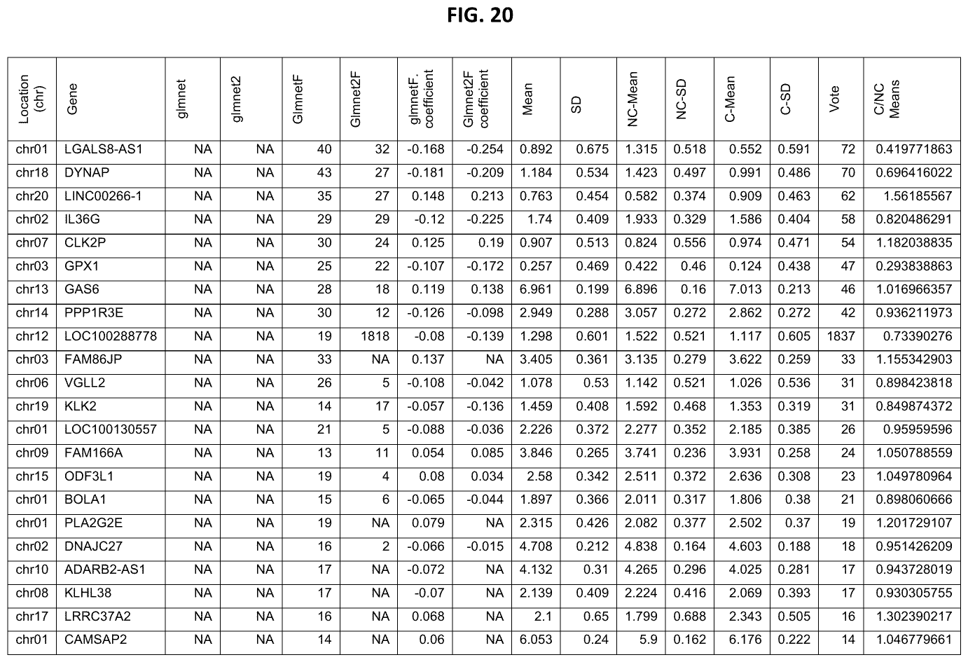

The final models fitted to the 65% most variable 5hmC gene features, using hyperparameter values determined from the training set data analysis, were fitted to the whole cohort of PDAC and non-cancer samples to yield models with 109 genes (elastic net) and 47 genes (lasso). The models were found to possess t-scores that are concordant both the Li et al. (2017) and Song et al. (2017) data sets (FIG. 18).

The experimental work detailed above focused on the discovery of cfDNA-specific hydroxymethylation-based biomarkers that can be employed in molecular diagnostic tests to detect pancreatic cancer non-invasively and at earlier stages than previously possible. The data discussed above and presented in the figures highlight the ability to detect differentially hydroxymethylated genes whose underlying biology shows association with both pancreas and cancer development as well as established trends in marked, known functional regions of the genome. Furthermore, by using either 5hmC signals from biologically significant genes or from regularized regression methods, predictive models can be built with AUC=0.94-0.96 with an external data set validation AUC=0.74-0.97 (elastic net models).

C. Conclusions:

The 5hmC signal was found to be enriched in gene-centric sequence types (promoter, exons, UTR and TTS), as well as transposable elements like SINEs (enriched) and LINEs (depleted) (FIGS. 3 and 4). These hydroxymethylation changes in functional regions have been reported in cfDNA from colorectal, esophageal, liver, and lung cancer (see Li et al. (2017), supra; Tian et al. (2018) Cell Res 5:597-600; Cai et al. (2018), “5-Hydroxymethylcytosines from Circulating Cell-free DNA as Diagnostic and Prognostic Markers . . . ,” bioRxiv (doi:https://doi.org/10.1101/424978), and Zhang et al. (2018) Genomics, Proteomics & Bioinformatics 16: 187-199); however, no PDAC-specific gains or losses in hydroxymethylation were observed in functional regions. In addition to enrichment and depletion of 5hmC in functional regions, there was a novel PDAC specific 5hmC increase in exons, TTS and 3′UTR and a 5hmC decrease in promoters and LINE elements (FIG. 5). In ES cells, the decrease of 5-hydromethylation in the promoter region has been shown to associate with gene transcription (see Szulwach et al. (2011) PLoS Genetics 7(6): e1002154). An increase in disease relevant transcription is implicitly supported in the above data by the 5hmC increase seen in gene-centric features and the apparent decreasing trend of 5hmC in promoter regions toward late stage PDAC (FIG. 6).

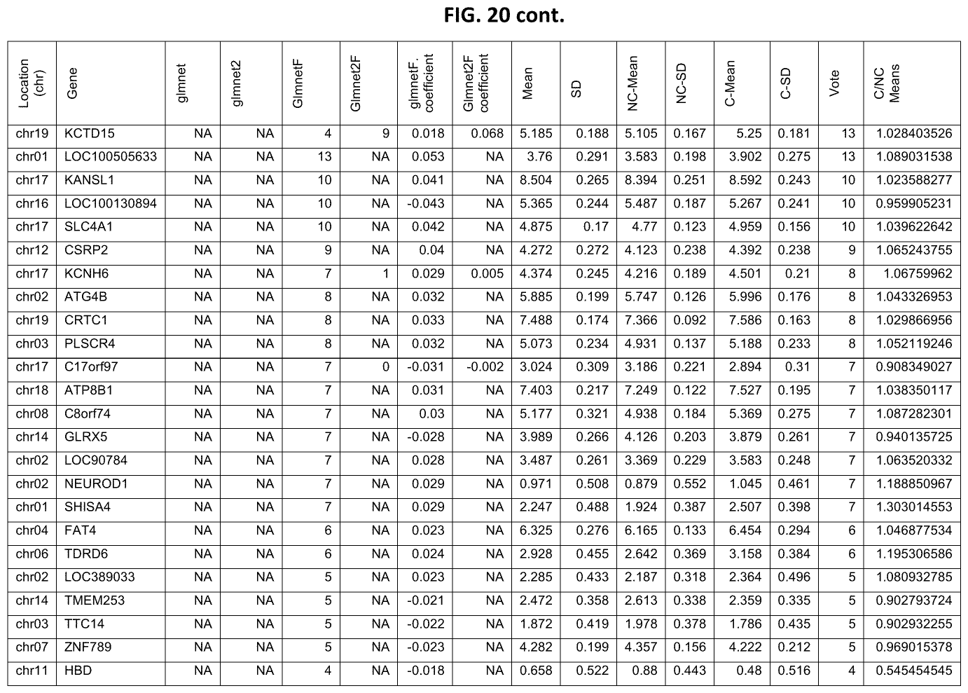

Dynamic changes in chromatin have been shown to control cell development and transition of cells with oncogenic potential; see Bernhart et al. (2016) Scientific Reports 6, Article number 37393. The PDAC-specific decrease of 5hmC in H3K4me3 loci appears to be coincident with a non-statistically significant increase of 5hmC in H3K4me1 (FIG. 7). These DNA hydroxymethylation patterns complement each other both in genomic location and the histone marks they occupy (FIGS. 8A and 8B), and also suggest disease-specific increases in gene transcription via chromatin modifications, given the known permissive transcriptional function associated with H3K4me3/me1. Also see FIG. 19, which illustrates in graph form the 5hmC occupancy levels at loci associated with H3K4me3, H3K4me1, and H3K27ac, and the similarity to an existing histone map from PANC-1 cell lines (LeRoy et al. (2013) Epigenetics & Chromatin 6:20).

An intriguing aspect of the precision of 5hmC patterns in these regions of known functional sequence suggest a widespread function for hydroxylation in the epigenetic control of transcriptional processes.

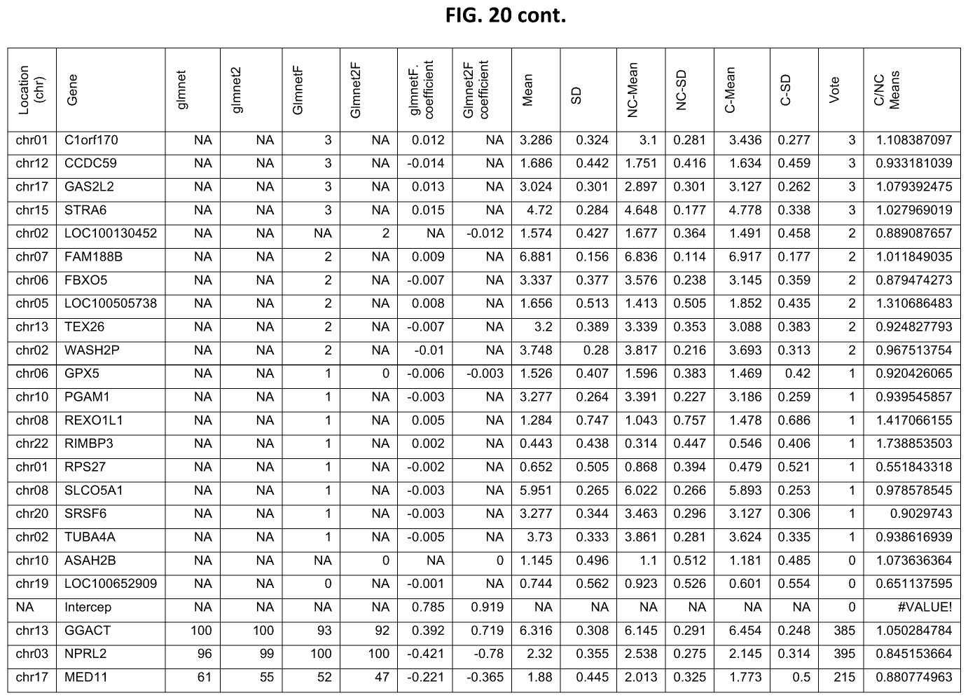

FIG. 20 provides hydroxymethylation biomarker data obtained using the methods documented in this example. The table of FIG. 20 identifies the genes by name and chromosome location and includes normalized values obtained with glmnet, glmnet2, glmnetF, and glmnet2F regularization methods; glmnetF and glmnet2F coefficients; mean and standard deviation; mean and standard deviation for the cancer cohort (identified as Mean-C and SD-C, respectively); mean and standard deviation for the non-cancer cohort (Mean-NC and SD-NC, respectively); vote, computed as the sum of the glmnetF and glmnet2F normalized values for each gene; and the ratio of cancer-to-non-cancer (C/NC) means.

Genes were identified whose increased 5hmC signals in highlighted pathways are implicated in liver cancer (Table 7). MSigDB does not currently contain pathways annotated for pancreatic cancer; see Subramanian et al. (2005), PNAS 102:15545-50. Two approaches were used for gene set enrichment analysis, either using genes with significantly decreased 5hmC or performing GSEA on all reporting genes. The results indicated that close to one third of immune system pathways were implicated in pancreatic cancer. Assuming the strong association between 5hmC extent and gene transcription, this result suggests that immune system function is decreased in PDAC patients. Inspection of individual genes that were either significantly increased or decreased in functional regions reveals genes implicated in normal pancreas development, for instance the transcription factors GATA4, GATA6, PROX1, ONECUT2, and also genes whose increased expression is implicated in cancer like YAP1, TEAD, PROX1, ONECUT2, ONECUT1, IGF1 and IGF2.

Accordingly, using genes whose 5hmC densities were found to be significantly changed in PDAC, with annotated relevant biology, it was possible to build regularized regression models whose performance matched model building using algorithm gene based selection. This provided confidence that the models used, whose performance was high (training AUC=0.94-0.96 with an external data set validation AUC=0.74-0.97), was measuring underlying biological signals relevant to PDAC. Despite the large number of significantly hydroxymethylated genes, the regularized regression models selected 100 genes or fewer. However, the fact that 13,180 significantly represented genes were detected provide evidence that other biological signals may also reside in the data set.

### EXAMPLE 2

Example 1 was repeated with an additional study group, Study Group 2, of 41 PDAC and 82 non-cancer subjects. The clinical characteristics of the cancer and non-cancer cohorts in Study Group 2 are set forth in Table 9.

TABLE 9

| | No Cancer | Cancer |
|---|---|---|
| Age⁺ | | |
| | 66.0 | 65.5 |
| Gender (%) | | |
| Male | 50 | 44 |
| Smoking History | | |
| Status (%) | | |
| Current | 5 | 12 |
| Former | 54 | 39 |
| Never | 41 | 49 |
| Stage | | |
| NA | NA | 5 |
| I | NA | 22 |
| II | NA | 29 |
| III | NA | 15 |
| IV | NA | 29 |

⁺mean of Non-Cancer and Cancer

The procedures documented in Example 1 were followed to generate the 611 hydroxymethylation biomarkers set forth in FIG. 21.

### EXAMPLE 3

Example 1 was repeated with a further study group, Study Group 3, of 53 PDAC and 53 non-cancer subjects. The clinical characteristics of the cancer and non-cancer cohorts in Study Group 3 are set forth in Table 10.

TABLE 10

| | No Cancer | Cancer |
|---|---|---|
| Age⁺ | | |
| | 66.0 | 66.4 |
| Gender (%) | | |
| Male | 44 | 53 |
| Smoking History | | |
| Status (%) | | |
| Current | 21 | 21 |
| Former | 32 | 32 |
| Never | 47 | 47 |
| Stage | | |
| NA | NA | 4 |
| I | NA | 21 |
| II | NA | 36 |
| III | NA | 11 |
| IV | NA | 28 |

⁺mean of Non-Cancer and Cancer

The procedures documented in Example 1 were followed to generate the 41 hydroxymethylation biomarkers set forth in Table 4, provided earlier herein, and in FIG. 22.

### EXAMPLE 4

The techniques and processes used to obtain the data set forth in this Example are similar to the techniques and processes detailed in Example 1, part A, but differ in several respects. As such, a complete Study Design and Methods section pertaining to this Example follows, as part A, below:

A. Study Design and Methods:

(i) Clinical Cohorts and Study Design:

A case-control study was performed using plasma obtained from subjects with or without pancreatic ductal adenocarcinoma, 38 in a non-cancer cohort and 41 in a cancer cohort, each of whom provided informed consent and contributed biospecimens in studies approved by the IRBs at participating sites in the United States. Plasma samples for the non-cancer cohort were obtained from subjects enrolled prospectively from five sites in the United States. Criteria for subject eligibility for inclusion in the analysis included age greater than or equal to 40 years for all subjects and no treatment with medication for any disease at the time of blood collection.

Cancer cohort: Plasma samples for the cancer cohort were obtained from subjects who had undergone management for PDAC in the United States. Criteria for subject eligibility: 1) no cancer treatment, e.g., surgical, chemotherapy, immunotherapy, targeted therapy, or radiation therapy, prior to study enrollment and blood specimen acquisition; and 2) a confirmed pathologic diagnosis of adenocarcinoma inclusive of all subtypes.

Non-cancer cohort: Subject exclusion criteria for the non-cancer cohort also included any of the following: a cancer diagnosis within prior six months; surgery or invasive procedure requiring general anesthesia within prior month; non-cancer systemic therapy associated with molecularly targeted immune modulation; concurrent or prior pregnancy within previous 12 months; history of organ tissue transplantation; history of blood product transfusion within one month; and major trauma within six months. Clinical data required for all subjects included age, gender, smoking history, and both tissue pathology and grade, and were managed in accordance with the guidance established by HIPPA to ensure subject privacy.

The clinical characteristics of the cohorts are set forth in Table 11:

TABLE 11

| | Non-Cancer | Cancer |
|---|---|---|
| Age⁺ | | |
| | 64.9 | 65.5 |
| Gender (%) | | |
| Male | 47.4 | 43.9 |
| Smoking History | | |
| Status (%) | | |
| Current | 13.2 | 12.2 |
| Former | 42.1 | 39.0 |
| None | 44.7 | 48.8 |
| Stage | | |
| I | NA | 24.4 |
| II | NA | 31.7 |
| III | NA | 14.6 |
| IV | NA | 29.3 |

⁺mean of Non-Cancer and Cancer groups.
Other values are percentages of each category in “Non-Cancer” and “Cancer” groups.

There were no statistically significant differences in subject age, gender or tobacco exposure between the two cohorts (Table 8). Early stage PDAC patients (Stages I & II) made up 56% of the total PDAC cohort.

(ii) Plasma Collection:

Plasma was isolated from whole blood specimens obtained by routine venous phlebotomy at the time of subject enrollment. For both cancer and non-cancer control subjects, whole blood was collected in Cell-Free DNA BCT® tubes according to the manufacturer's protocol (Streck, La Vista, Nebr.). Tubes were maintained at 15° C. to 25° C. with plasma separation performed within 24 h of phlebotomy by centrifugation of whole blood at 1600×g for 10 min at RT, followed by transfer of the plasma layer to a new tube for centrifugation at 16,000×g for 10 min. Plasma was aliquoted for subsequent cfDNA isolation or storage at −80° C.

(iii) Cell-Free DNA Isolation:

Cell-free DNA was isolated using the QIAamp Circulating Nucleic Acid Kit (QIAGEN, Germantown, Md.) following the manufacturer's protocol excepting the omission of carrier RNA during cfDNA extraction. Four milliliter plasma volumes were lysed for 30 minutes prior to collection of nucleic acids; all cfDNA eluates were collected in a volume of 60 μl buffer. All cfDNA eluates were quantified by Bioanalyzer dsDNA High Sensitivity assay (Agilent Technologies Inc, Santa Clara, Calif.) and Qubit dsDNA High Sensitivity Assay (Thermo Fisher Scientific, Waltham, Mass.) to ensure the absence of contaminating high molecular weight DNA emanating from white blood lysis.

(iv) Tissue and Genomic DNA Processing:

All tissue samples were stored in H media for the interval between surgical resection and laboratory processing. Each sample was weighed and aliquoted into sections of approximately 35 mg. Each resulting subsection was briefly incubated on dry ice, then homogenized in 500 μl RLT Buffer Plus using a Tissue Lyser LT (QIAGEN Germantown, Md.) at 50 Hz for two minutes. Resulting homogenates were stored at −80 C until DNA extraction. Genomic DNA was extracted using DNeasy Blood & Tissue Kit (QIAGEN Germantown, Md.) according to manufacturer instructions. Genomic DNA eluates were quantified using the Qubit dsDNA High Sensitivity assay (Thermo Fisher Scientific, Waltham, Mass.) and stored at −20° C. until further processing. Prior to sequencing library construction, genomic DNA was fragmented to a modal 150 base pair size using an ME220 focused ultrasonicator (Covaris, Woburn, Mass.), Modal fragmented DNA sizes were verified using the TapeStation 2200 dsDNA high sensitivity assay (Agilent Technologies, Santa Clara, Calif.) and quantified as described above prior to commencing library construction.

(v) 5-Hydroxymethylcytosine (5hmC) enrichment assay: 5hmC-enriched libraries were prepared as described in Example 1, section (iv) of Part A.

(vi) DNA sequencing and alignment: DNA sequencing was performed according to manufacturer's recommendations with 75 base-pair, paired-end sequencing using a NextSeq550 instrument with version 2 reagent chemistry (Illumina, San Diego, Calif.). Data was collected using NextSeq System Suite 2.2.04. Twenty-four libraries were sequenced per flowcell. Raw data processing and demultiplexing was performed using the Illumina BaseSpace Sequence Hub to generate sample-specific FASTQ output. Sequencing reads were aligned to the hg19 reference genome using BWA-MEM with default parameters (Li et al. (2010) Bioinformatics 26: 589-595), supra. Sequencing data quality was assessed using picard (Institute, B. (2019) “Picard Toolkit.” Broad Institute, GitHub Repository, http://broadinstitute.github.io/picard/; Broad Institute).

(vii) Peak detection: BWA-MEM read alignments were employed to identified regions or peaks of dense read accumulation that mark the location of a hydroxymethylated cytosine residue in a CpG context. Prior to identifying peaks, BAM files containing the locations of aligned reads were filtered for poorly mapped (MAPQ <30) and not properly paired reads. using samtools and HTSlib (Robinson et al. (2011), supra). 5hmC peak calling was carried out using MACS2 (https://github.com/taoliu/MACS) with a p-value cut off=1.00e-5. Identified 5hmC peaks residing in “blacklist regions” as defined elsewhere (https://sites.google.com/site/anshulkundaje/projects/blacklists) and residing on chromosomes X, Y and mitochondrial genome were also removed using bedtools. Computation of genomic feature enrichment overlap 5hmC peaks were performed using the HOMER software (http://homer.ucsd.edu/homer/) with default parameters.

(viii) Chromatin Immunoprecipitation:

Chromatin immunoprecipitations of H3K4me1, H3K4me3, H3K27ac, H3K36me3, H3K9me3 and H3K27me3 in primary PDAC tumor tissues were performed at Active Motif (Carlsbad, Calif.). Briefly, tumor tissues were homogenized, then sonicated and subjected to chromatin immunoprecipitation with antibodies specific to chromatin modifications mentioned above (anti-H3K4me1 Active Motif 39297, 4 μl per IP; anti-H3K4me3 Active Motif 39159, 3 μl per IP; anti-H3K9me3 Abcam ab8898, 5 μl per IP; anti-H3K27ac Active Motif 39133, 4 μl per IP; anti-H3K27me3 Active Motif 39155, 4 μl per IP; and anti-H3K36me3 Active Motif 61101, 4 μl per IP). Immunoprecipitated DNA was isolated, then subjected to library preparation and was subsequently sequenced. Reads were mapped using BWA-MEM, then filtered for quality reads as described above. Peaks for each histone modification was determined using MACS2 with default parameters for H3K4me1, H3K4me3 and H3K27ac; while—broad option was used for H3K9me3, H3K27me3 and H3K36me3. ChromHMM was run with all six histone ChIPseq mentioned above (Ernst et al. (2012) Nat Methods 9: 215-216). Normalized 5hmC read densities were visualized as line plots using SeqPlots (Stempor et al. (2016), “Interactive software for exploratory data analyses, pattern discovery and visualization in genomics,” Wellcome Open Res 1(14):46). Genomic regions were visualized using IGV (Robinson et al. (2011), “Integrative genomics viewer,” Nat. Biotechnol. 29 (24).

(ix) Differential representation analysis: To identify gene bodies with differential representation between the PDAC and the non-cancer groups, we again followed the RNA-Seq workflow outlined in Law et al., supra, including many of the preliminary QC steps. The analysis included data pre-processing by adopting the following workflow: (i) transforming the data from raw counts to log₂ (counts per million), (ii) removing genes that were found to be weakly represented, (iii) normalizing the gene representation distributions, and (iv) performing unsupervised clustering of samples. To accomplish differential representation analysis, we applied the following steps: (i) creating a design matrix to contrast PDAC versus non-cancer cohorts, (ii) removing heteroscedasticity from the data, (iii) fitting regression models for the comparison of interest, PDAC vs non-cancer, and (iv) examining the number of differentially represented genes. In most of these steps, the default settings were used when appropriate. To remove weakly represented genes, we excluded genes that did not have greater than 3 counts per million reads in at least 20 samples. This filter excluded roughly 12% of the genes. For the identification of the significantly differentially represented regions, the method of Benjamini et al., cited supra, was used to obtain p-values adjusted for multiple comparisons.

B. Results:

(i) Genomic Distributions of 5hmC Densities in PDAC and Non-Cancer Cohorts:

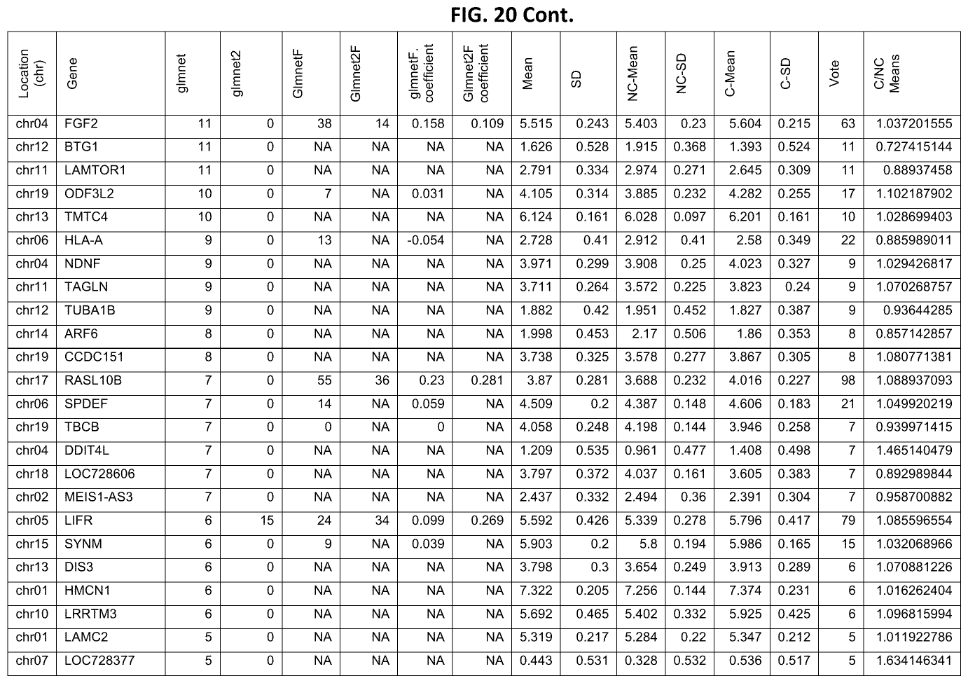

To gain an understanding of the genomic regions associated with hydroxymethylation, 5hmC enriched loci were first determined, as measured by increased read density and subsequent detection as peaks by MACS2. The vast majority of 5hmC loci occur on average in non-coding regions of the genome, over introns and intergenic space some of which overlap with SINE repetitive elements, with no preferential distribution in the PDAC or non-cancer cohort (FIG. 39). However, 5hmC residues were not particularly enriched over these regions relative to the genome background (FIG. 23). Instead, 5hmC enrichment was observed over genic features, most significantly in promoters, 3′UTRs, exons, transcription termination sites (TTS) and SINE repetitive elements that are located in gene rich regions, as measured by increased relative fold change compared to the genome background (FIG. 23). These results indicate that 5hmC in cfDNA is preferentially enriched in genic regions, consistent with previously published reports (see, e.g., Szulwach et al. (2011) PloS Genetics 7 (https://doi.org/10.1371/journal.pgen.1002154).

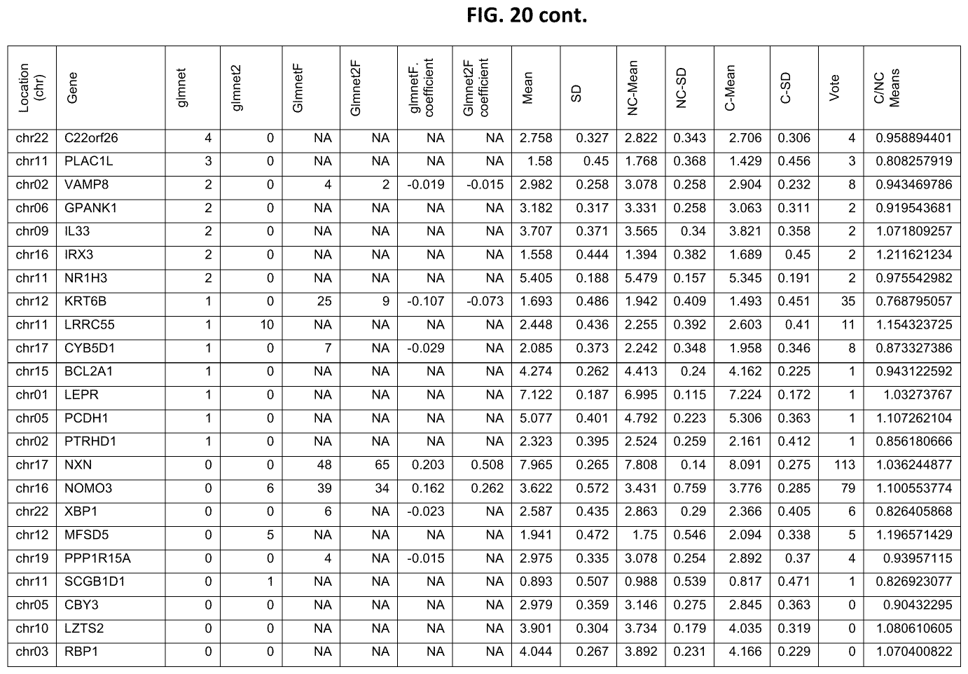

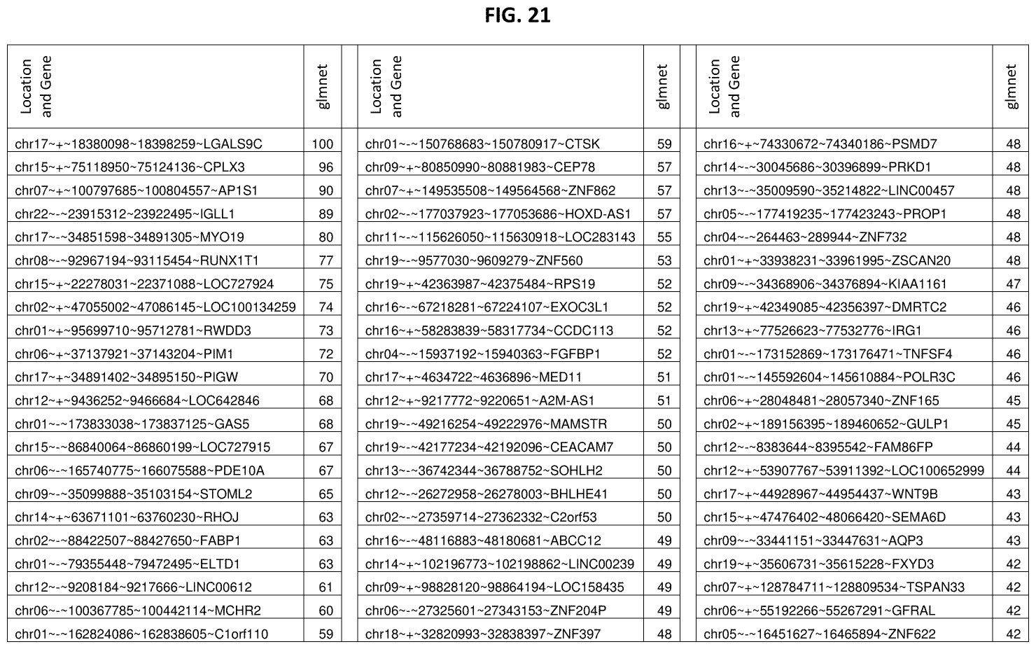

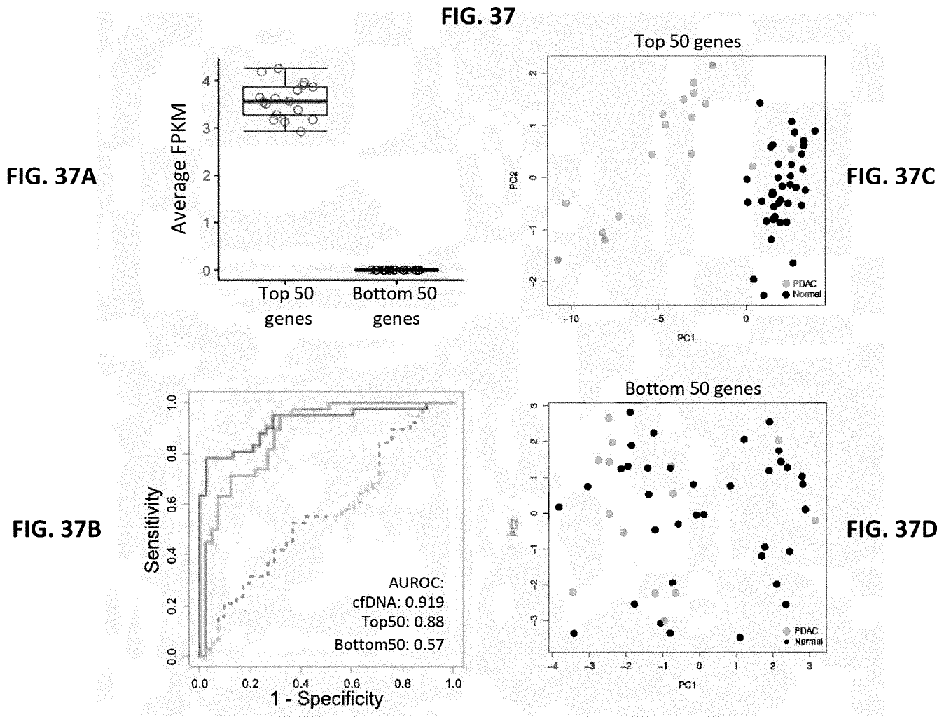

Comparison of 5hmC peaks in the PDAC cohort relative to the non-cancer cohort revealed significant differences. First, peaks detected per million reads in PDAC cfDNA cohort were significantly less than in the non-cancer cohort (FIG. 24). Decreased number of peaks suggests a global decrease in 5hmC in PDAC, consistent with previous reports investigating tissue samples (see, e.g., Yang et al. (2012), “Tumor development is associated with decrease of TET gene expression and 5-methylcytosine hydroxylation,” Oncogene 32: 663-9). In enrichment over several genomic features. 5hmC peak enrichment was increased in PDAC over 3′UTR, TTS and introns whereas it decreased over promoters (FIG. 25). These global changes, observed in a statistically significant manner in each cohort, were also detected in various cancer stages, including early stage cancers (FIG. 40).

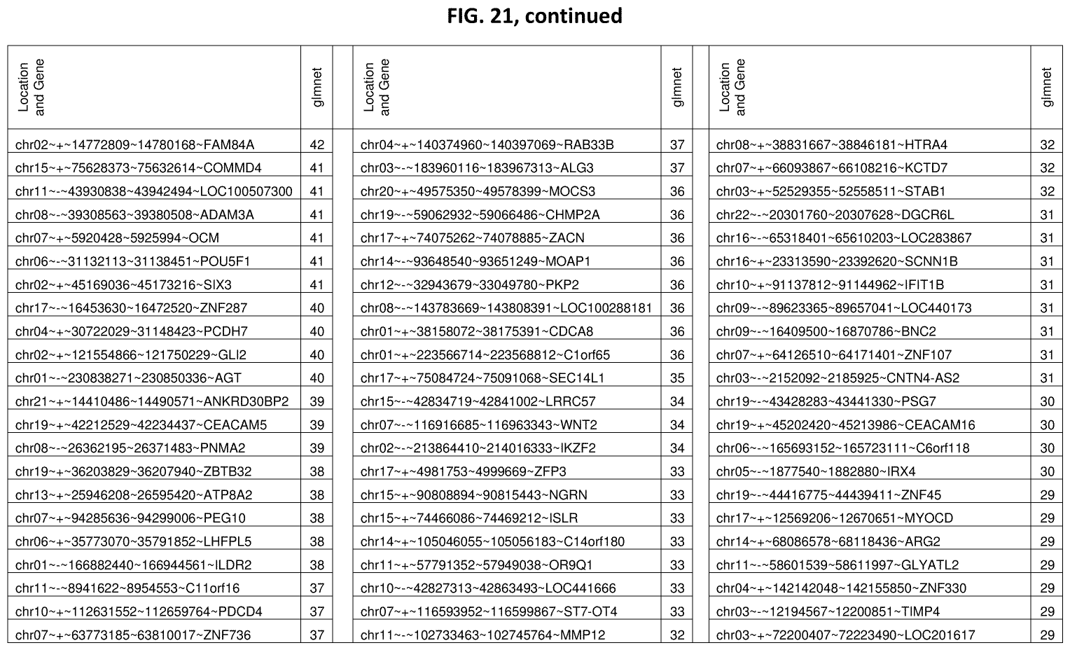

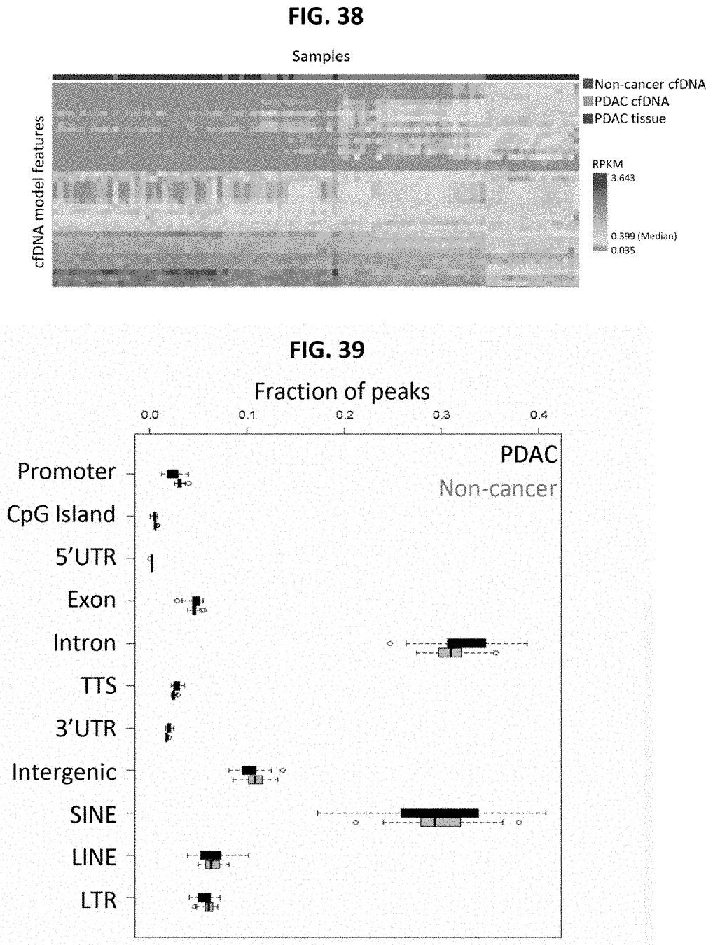

5hmC occupancy, and its associated changes in PDAC with respect to chromatin state, were investigated next. For this purpose, we first generated histone maps of primary tumor tissues obtained from two different PDAC patients with chromatin immunoprecipitation followed by sequencing. Targeting post-translational modifications such as methylation and acetylation on histone H3 that define various functional regions, we segmented the chromatin into 15 chromatin states that identify actively transcribed and silent regions, as well as regulatory regions, using chromHMM (Ernst et al. (2012) Nat Methods 9: 215-216) (FIG. 26). 5hmC profiles generated from 17 PDAC tumor tissues from different PDAC patients overlapped most with the active TSS as well as active enhancer regions (FIG. 41A), indicating that 5hmC marks regulatory regions with active transcription. Comparison of 5hmC occupancy in PDAC cfDNA and non-cancer cfDNA cohorts revealed statistically significant differences in these chromatin regions, with decreased 5-hydroxymethylation in PDAC cohort over active TSS and flanking TSS regions, while increased 5-hydroxymethylation was observed over weakly transcribed regions (FIG. 26). 5hmC decrease over H3K4me3-marked active TSS sites was observed across all cancer stages (FIG. 41B and FIG. 41C). Overall, differential cfDNA hydroxymethylation over different chromatin contexts identified in tumor tissue suggests that elements of epigenetic dysregulation in cancer cells can be picked up in the cfDNA 5hmC signal.

(ii) Identification of Disease Specific Genes from Plasma Samples:

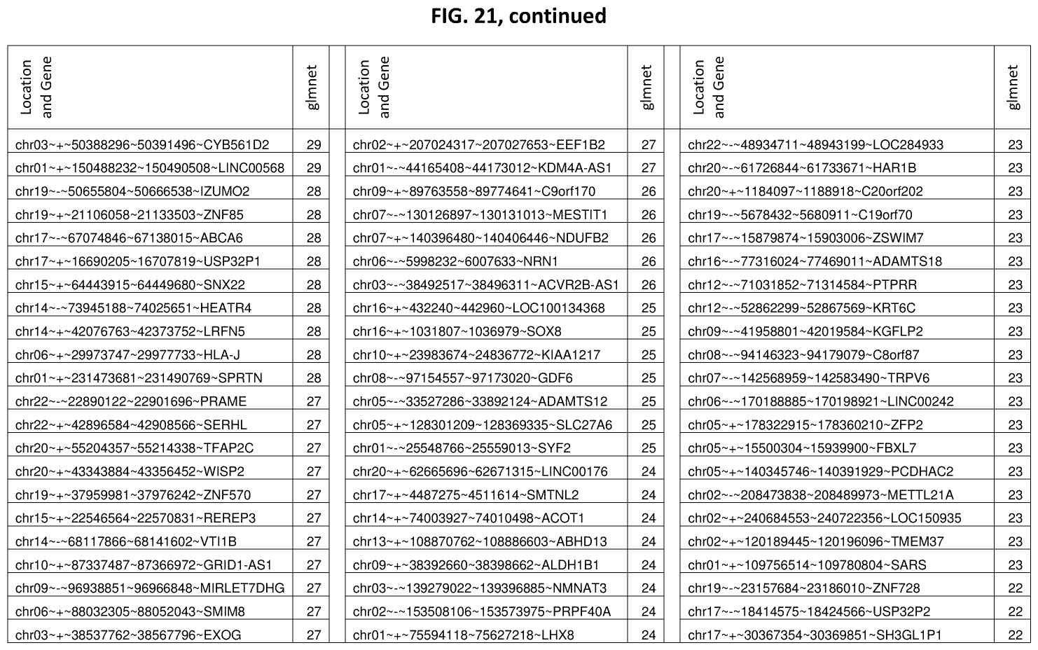

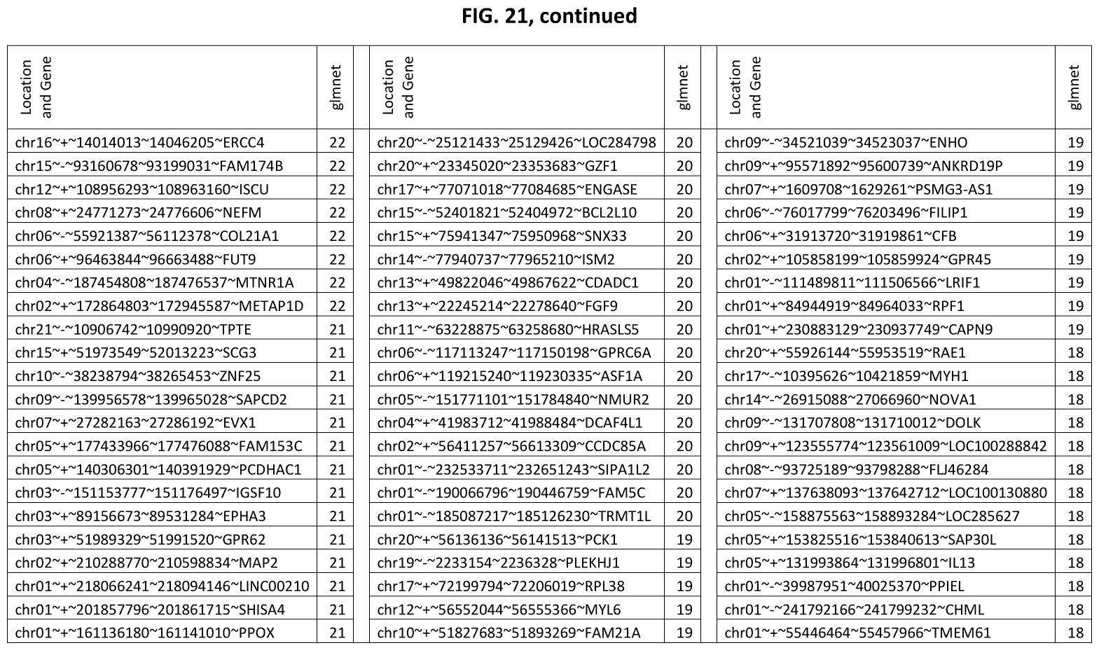

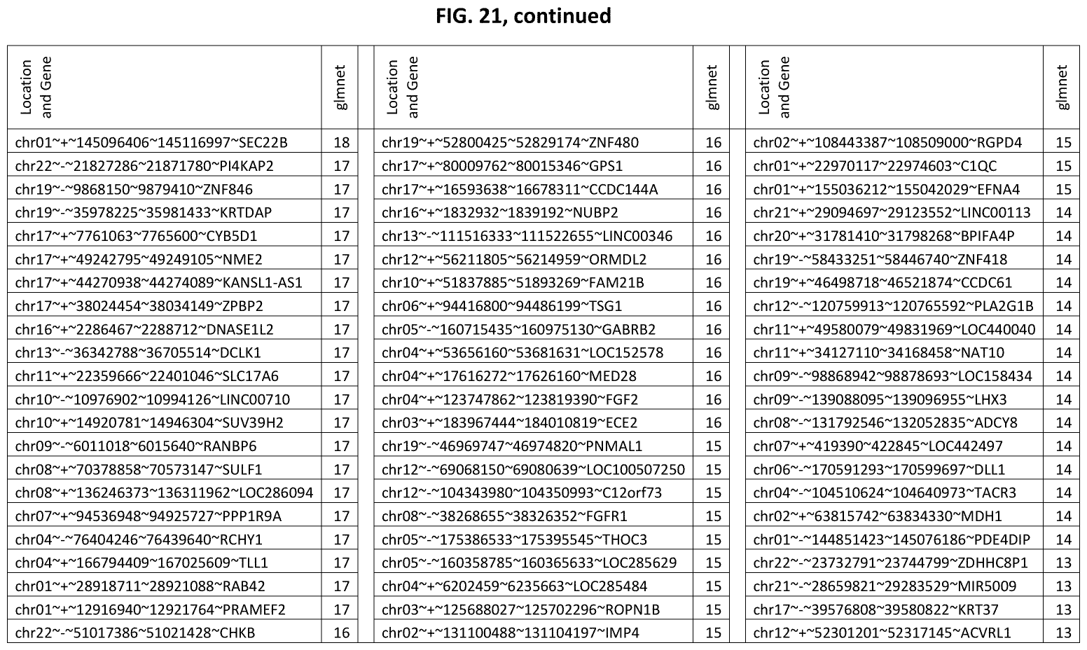

Differential analysis of 5hmC densities in genes using an adjusted p-value threshold of 0.05, revealed 5,700 hyper-hydroxymethylated and 6,155 hypo-hydroxymethylated genes in PDAC compared to non-cancer samples (FIG. 27). Further filtering of this gene set using a more stringent criteria (absolute fold change ≥1.5 and average log₂ CPM ≥3.5) resulted in 577 upregulated and 217 downregulated genes. As also found in Example 1, among the genes with increased 5hmC density in PDAC were those related to pancreas development (GATA4, GATA6, PROXI, ONECUT1, MEIS2) and/or implicated in cancer (YAP1, TEAD1, PROX1, ONECUT2, ONECUT1, AND IGF1) (FIG. 28). Inspection of the MSigDB for cancer relevant gene sets, C6. Oncogenic signatures and C4. Cancer modules, enriched among the 577 genes with increased 5hmC densities revealed a preponderance of gene sets that are upregulated in both KRAS and TP53 mutant cancers (FIG. 29). These genes were also enriched in targets of transcription factors known to be involved in PDAC oncogenesis or metastasis, like NFAT and FOXA.

In contrast, the most significantly downregulated 217 genes in PDAC cfDNA cohort was enriched for gene sets that are downregulated in KRAS mutant cells as well as immune response and whole blood genes (FIG. 29). These results are consistent with PDAC tumor DNA representation in cfDNA isolated from PDAC patients, whereas non-cancer cfDNA is known to be predominantly composed of DNA originating from various blood cells.

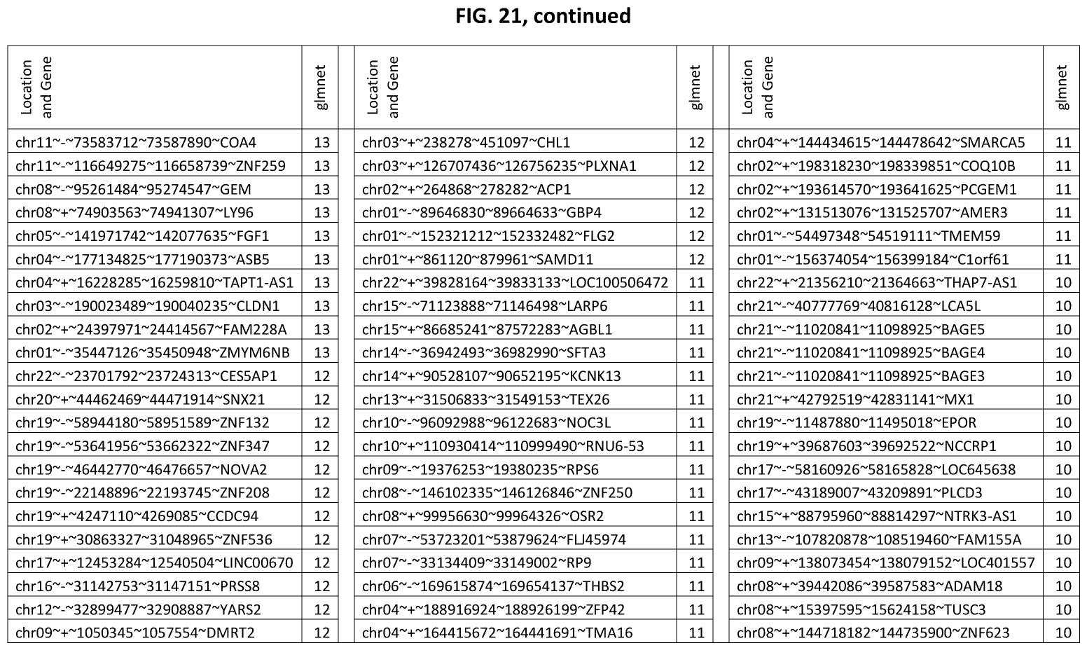

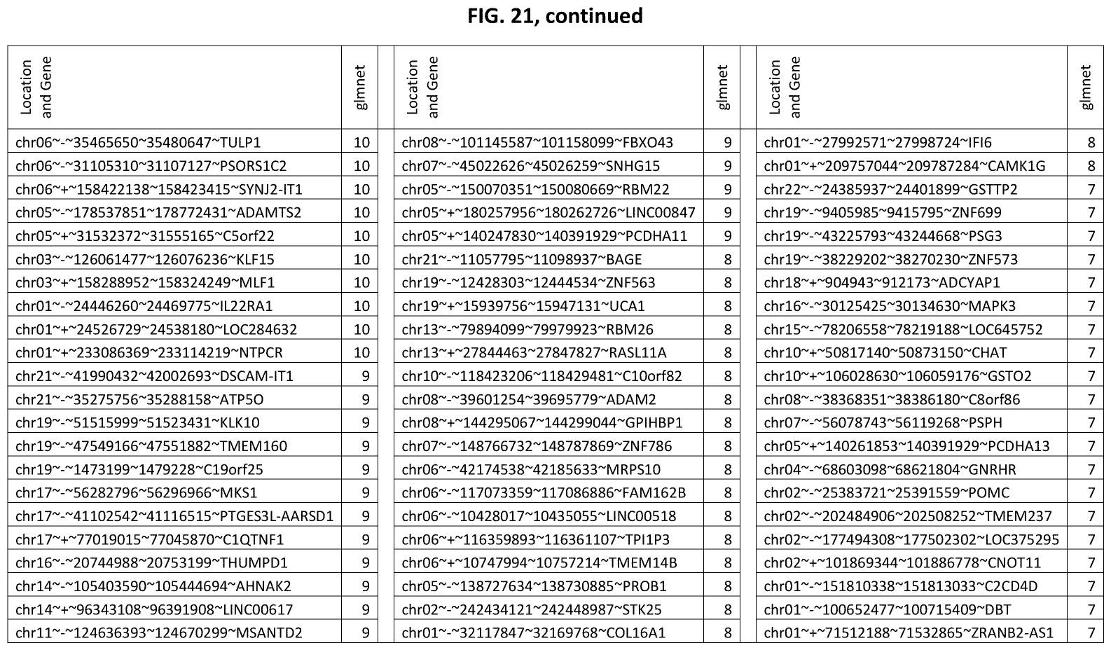

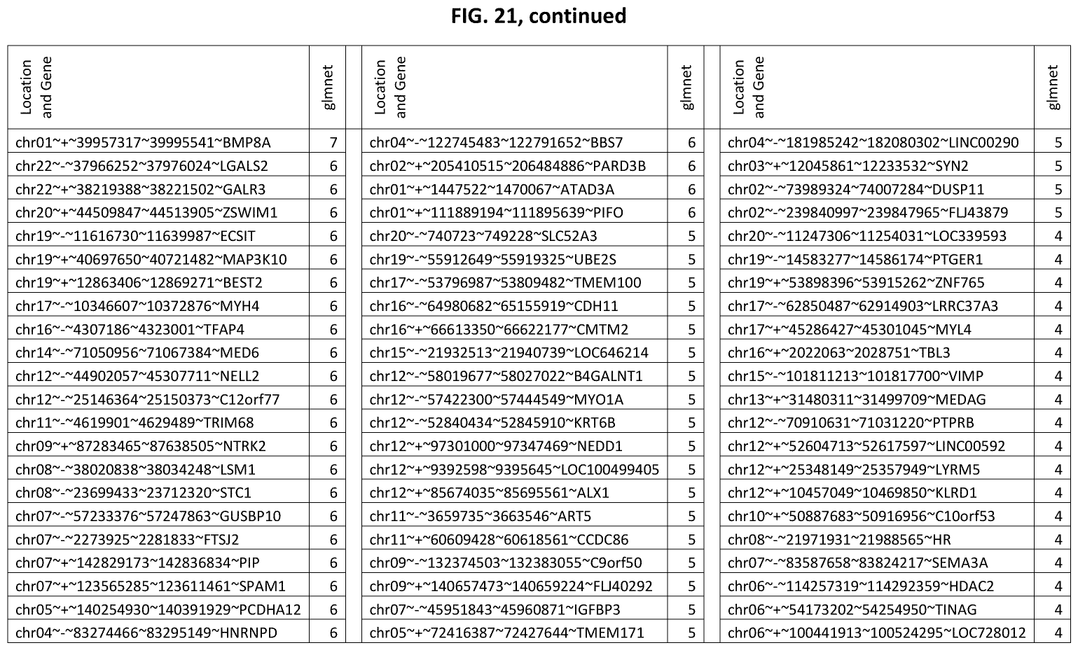

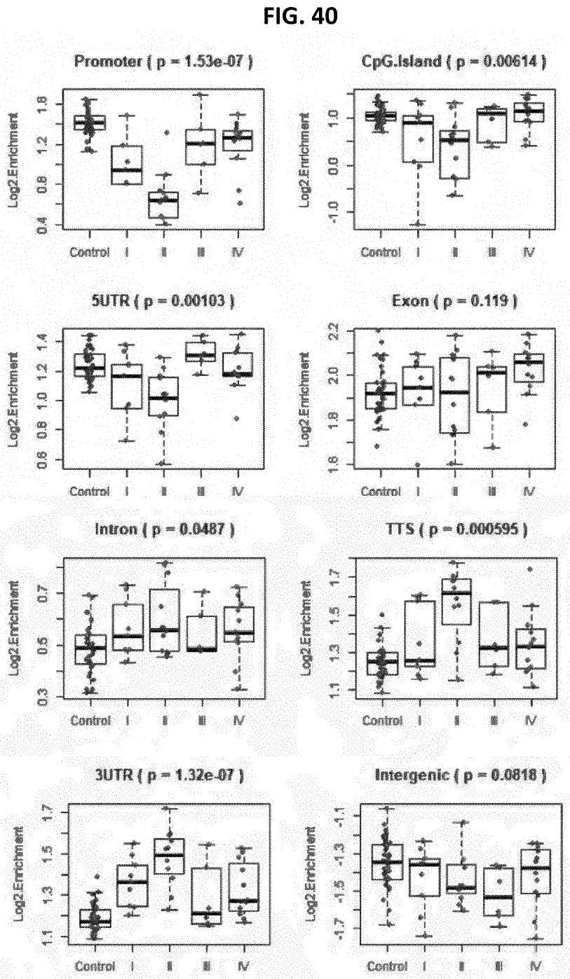

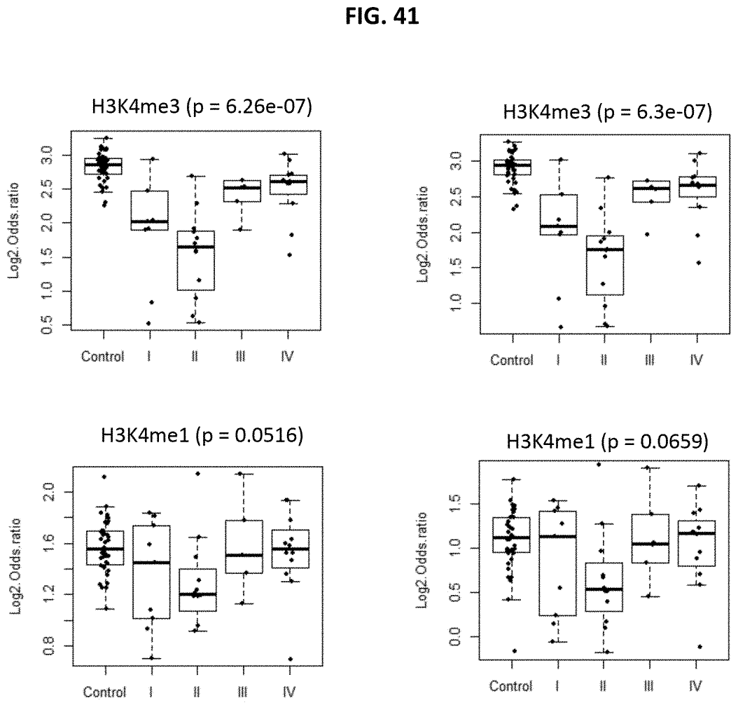

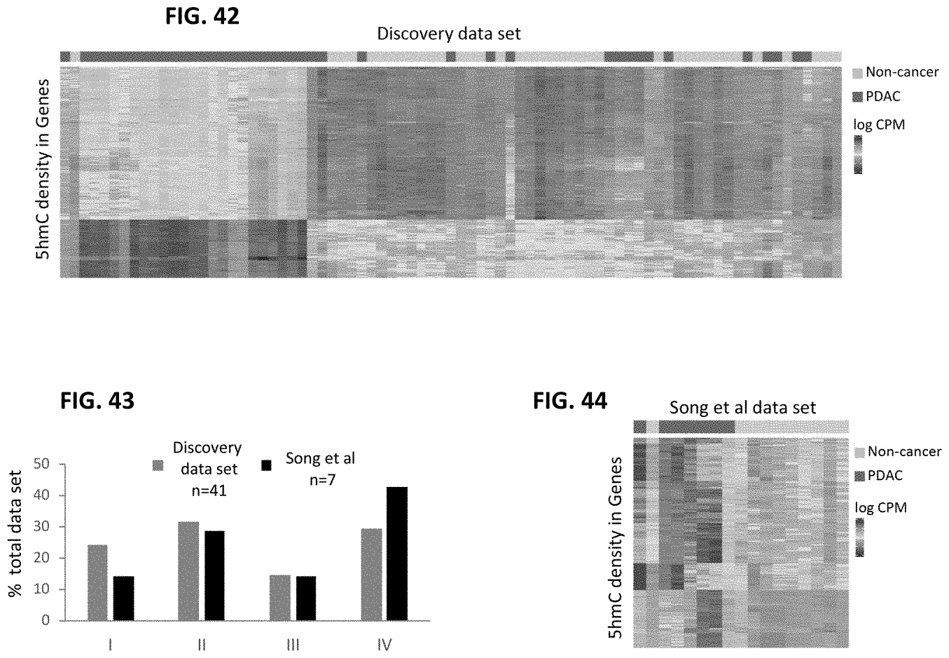

Multidimensional scaling (MDS) analysis using either the 11,855 genes with high variation in 5hmC counts (FIG. 30A) or the 794 genes filtered at the extremes of 5hmC representation in PDAC (FIG. 30B), reveal partitioning of the PDAC samples from the non-cancer equally well. We then tested the partitioning of a previously published dataset, Song et al. (2017), using the differentially represented genes we identified. The data set, despite small cohort size, had a similar cancer stage distribution as our discovery data set (FIG. 43). Furthermore, hierarchical clustering using the 794 genes revealed better partitioning of the 5hmC data from PDAC and non-cancer cfDNA from Song et al. (2017) (FIGS. 42 and 44). Consistently, PDAC samples in Song et al. (2017) could be separated from non-cancer samples using these 794 genes as shown by the MDS plot (FIG. 30C). In summary, we have been able to identify a differentially represented gene set whose biological functions are congruent with both pancreatic development and cancer. Furthermore, 5-hydroxymethylation densities of these genes alone enable distinction of PDAC from non-cancer.

(iii) Predictive Models for Detection of PDAC in cfDNA:

We performed regularized logistic regression analysis in order to determine whether gene-based features are present in the PDAC and non-cancer cohorts that can enable the classification of patient samples. We employed top 65% genes with the most variable 5hmC density for model selection. Elastic net was utilized as the regularization method. Other modeling approaches, such as random forest, support vector machines and neural networks, were explored in a preliminary analysis and were found to have inferior cross-validated performance on the training data (data not shown).

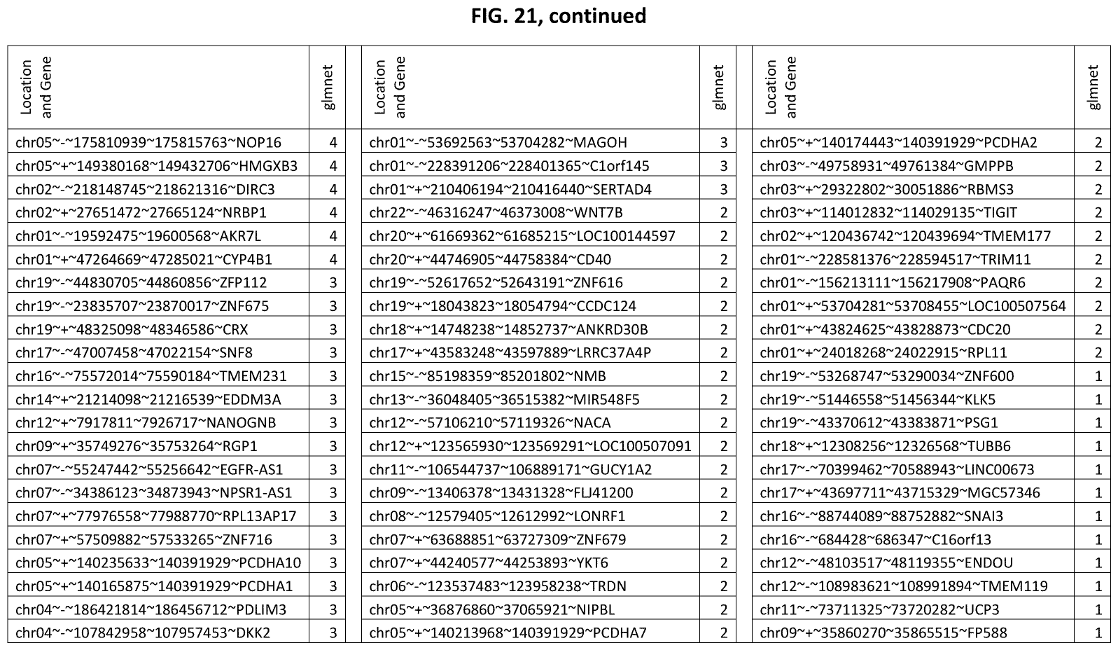

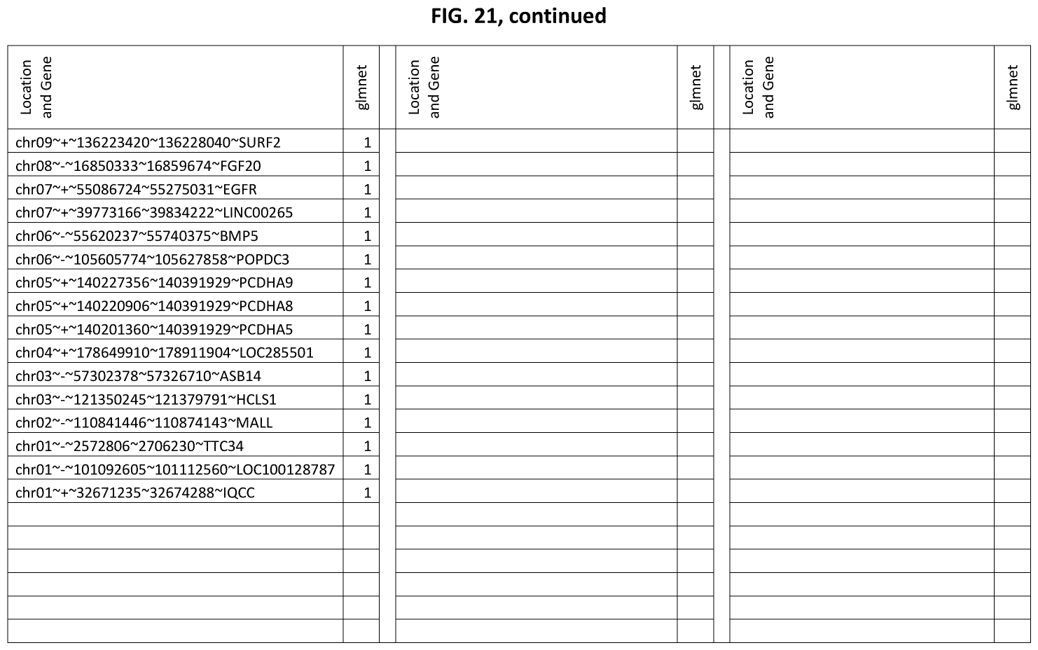

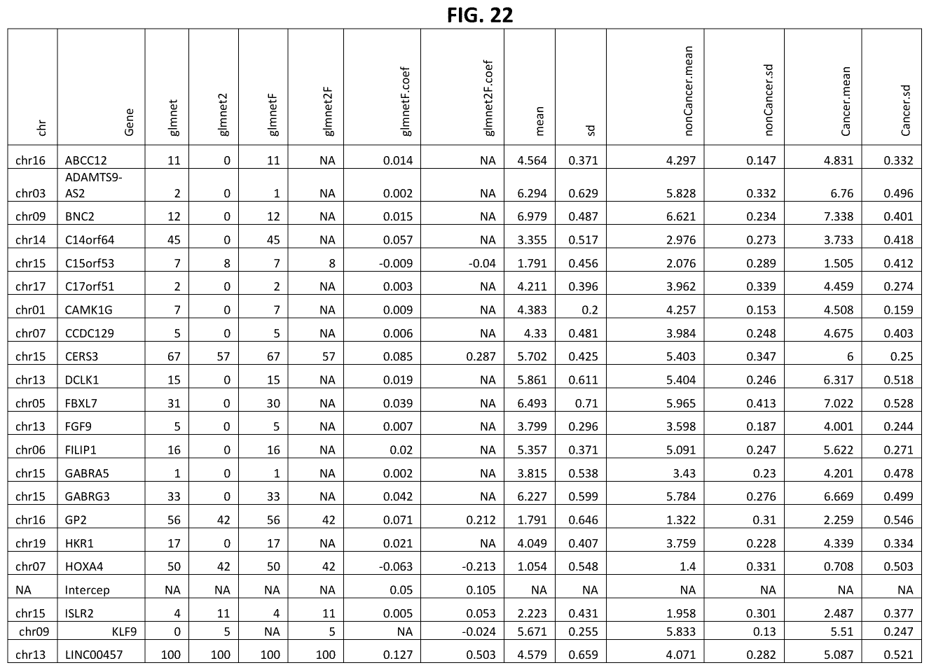

As noted previously, the elastic net regularization method requires specifying hyperparameters that control the level of regularization used in the fit. These hyperparameters were selected based on out-of-fold performance on 30 repetitions of 5-fold cross-validated analysis of the training data. Out-of-fold assessments were based on the samples in the left-out fold at each step of the cross-validated analysis. The training set yielded an out-of-fold performance metric, area under the ROC curve (AUC), of 0.919 (FIG. 31). The distribution of probability scores for each sample indicated few false negatives in the PDAC cohort and few false positives in the non-cancer cohort (FIG. 32). Inspection of features that were selected by the predictive model revealed cancer relevant genes such as the cancer antigen family gene BAGE5 (Benjamini et al., supra) and transcriptional co-repressor RUNX1T (Morgan & Shilatifard (2015), “Chromatin signatures of cancer,” Genes & Development 29: 238-249). On the other hand, model features that were down regulated in PDAC cfDNA compared to non-cancer cohort were enriched for immune/blood cell relevant genes such as SLFN14 which is important for platelet formation and CD22 which is expressed in B cells (FIG. 33) (see Calo and Wysocka (2013), “Modification of Enhancer Chromatin: What, How, and Why?” Molecular Cell 49: 825-837; and Raphael et al. (2017) Cancer Cell 32: 185-203e.13).

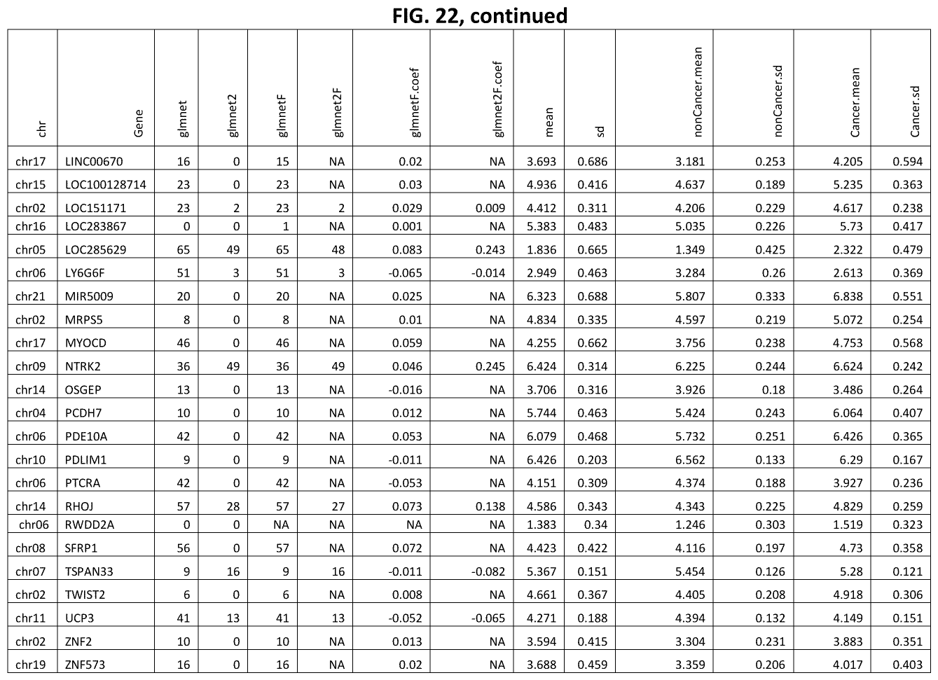

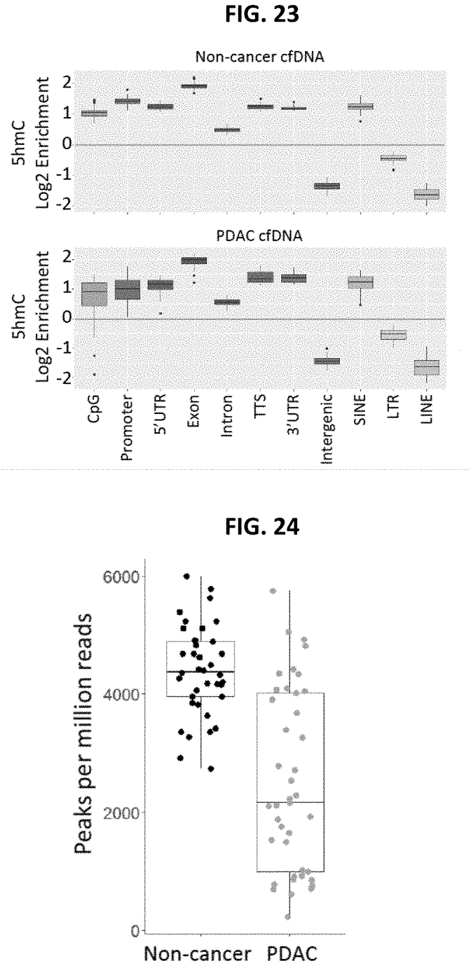

Next, the trained model was tested on two independent validation sets of patient samples. The first validation dataset was generated internally (23 PDAC and 205 non-cancer), yielding a classification performance AUC of 0.921 (FIG. 34). A second validation set included pancreatic cancer and non-cancer samples from Song et al. (2017) (pancreas subtype specified as adenocarcinoma, 7 pancreas cancer, 10 non-cancer). The validation set exhibited a performance AUC of 0.943 (FIG. 35).

(iv) Correlation of Prediction Performance with CA 19-9:

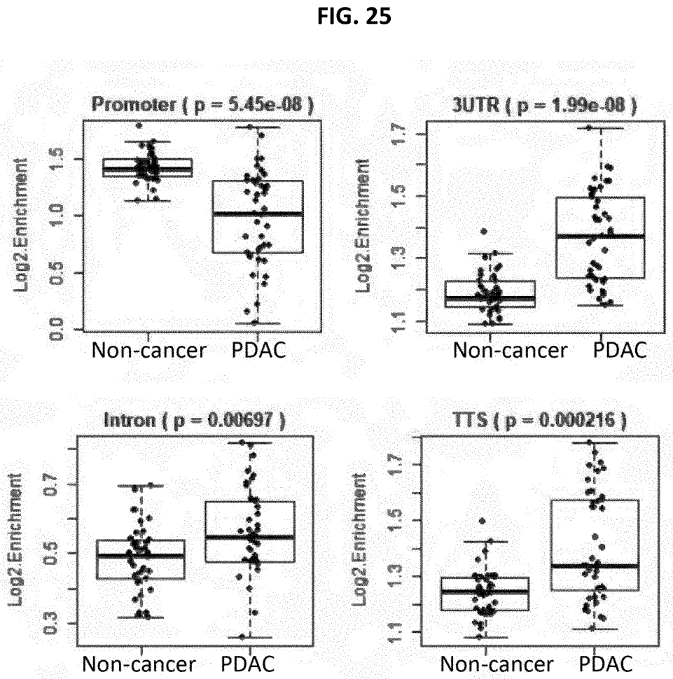

Next, the performance of 5hmC-based prediction probabilities was investigated respect to plasma levels of CA19-9, which is a clinically relevant biomarker in pancreatic cancer. CA19-9 was detected from plasma by electrochemiluminescent immunoassay (Roche) at Arup Laboratories. Despite being one of the most clinically utilized PDAC biomarkers, it is well established that CA19-9 levels in blood have challenges associated with specificity and sensitivity. Consistent with these previous observations, we found that CA19-9 level was abnormally high in one non-cancer sample (200 U/ml) and was within normal range for some Stage I, Stage II and even Stage III PDAC samples (FIG. 36). Interestingly, high probability scores calculated by our predictive model allowed detection of these early stage, namely Stage I and Stage II PDAC samples, that had low CA19-9 levels. On the other hand, the probability scores of some PDAC samples were low despite high CA19-9 levels. Taken together, these results suggest that 5hmC signals can significantly improve detection over existing methods for both sensitivity and specificity, particularly for early stage PDAC.

(v) Prediction Performance with Tissue-Derived Features:

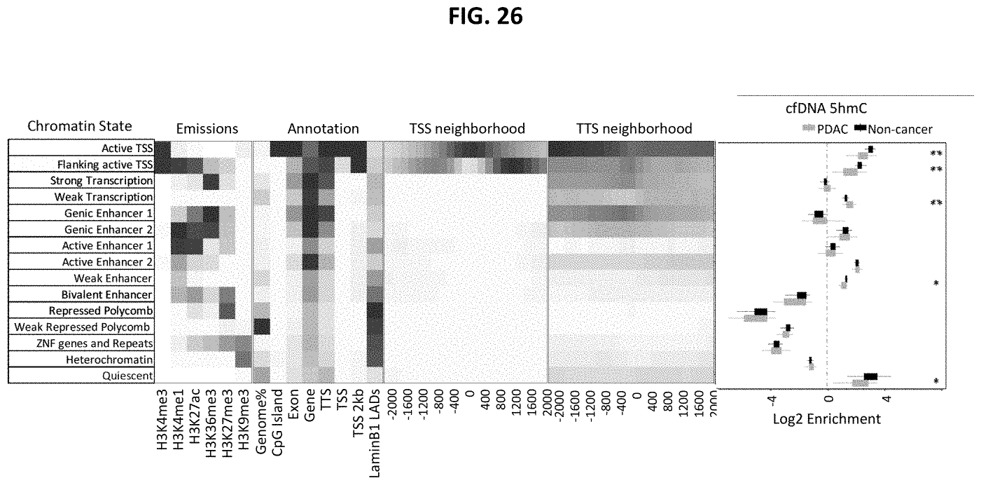

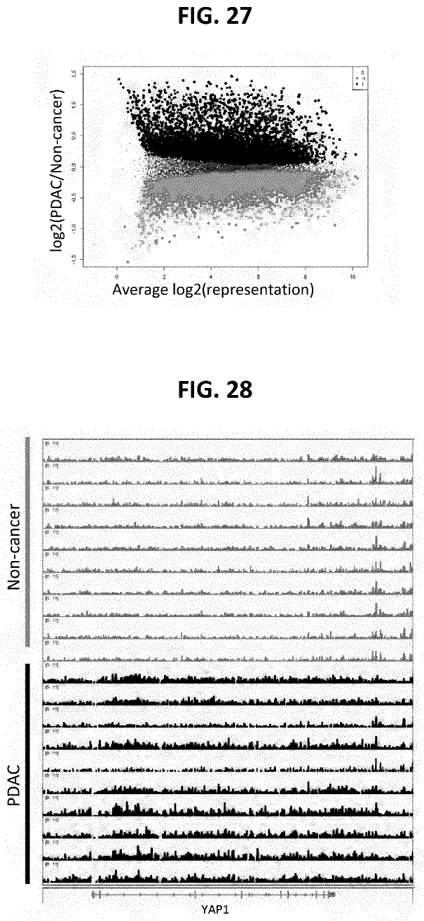

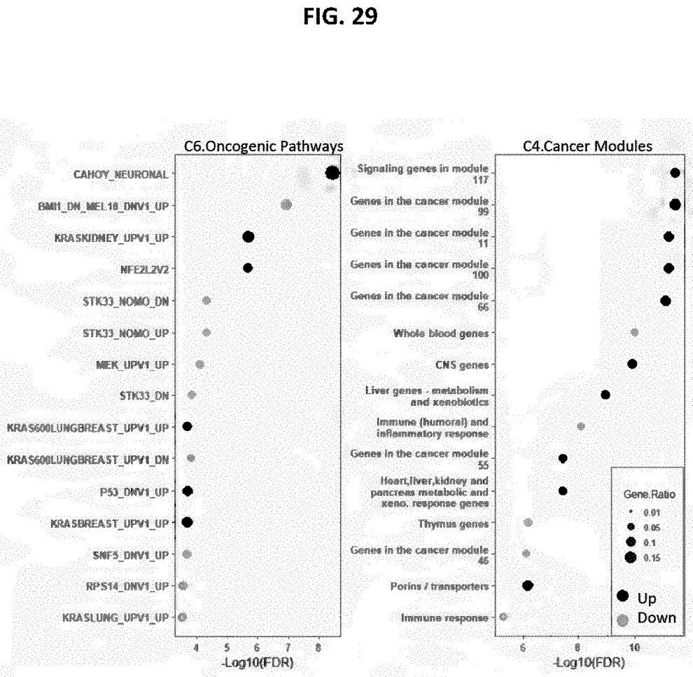

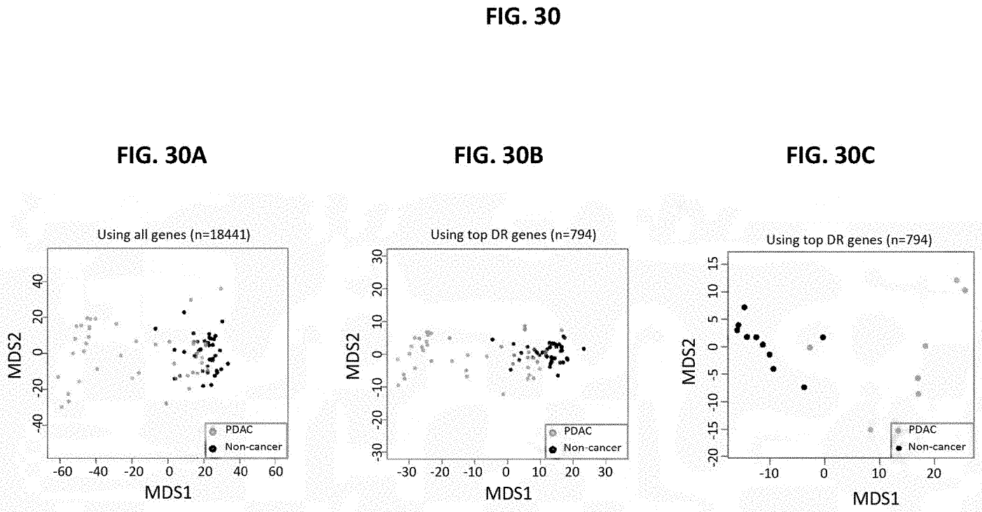

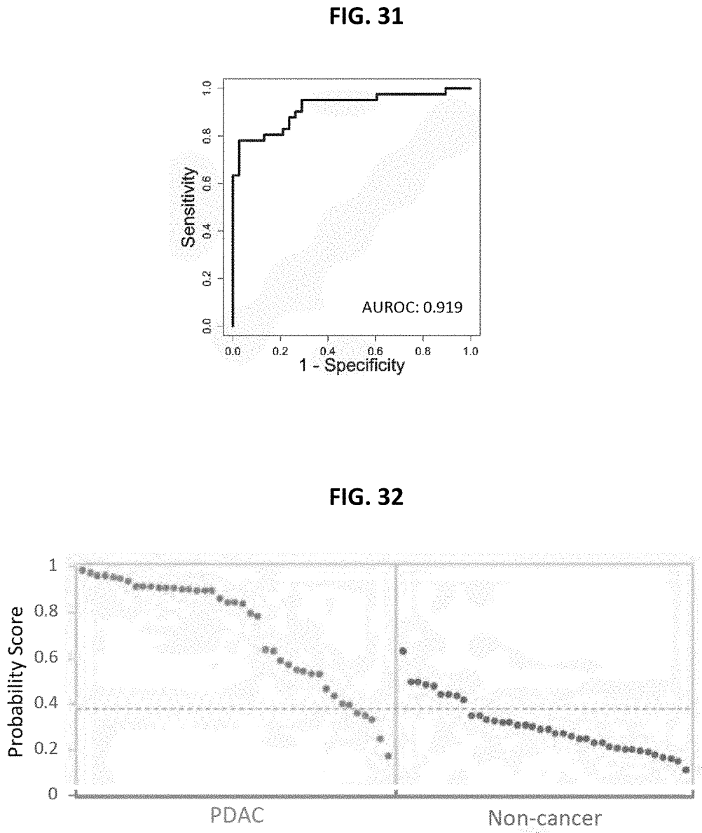

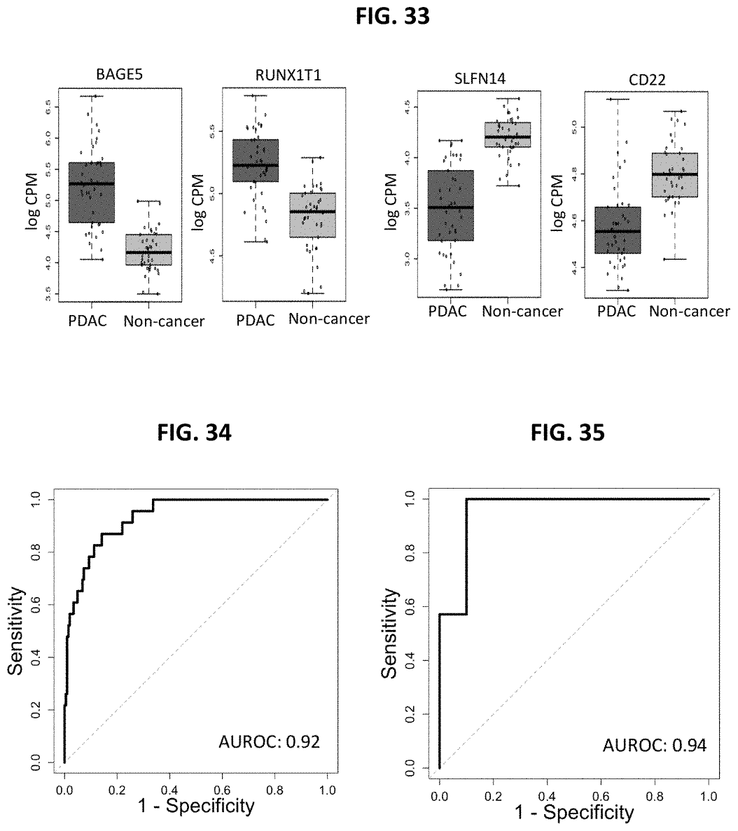

Next, we wanted to explore whether we can detect 5hmC based features that originate from tumor in cfDNA. For this purpose, we first profiled 5hmC in 17 PDAC tumor tissues. We then ranked all the genes based on FPKM values in these tissue profiles and took two gene sets; one representing the highest level of 5hmC occupancy (top 50 genes) and another set that represents the lowest levels of 5hmC occupancy (bottom 50 genes) in PDAC tissue (FIG. 37A). PDAC tissue-derived top 50 genes can separate non-cancer cfDNA from PDAC cfDNA samples well (FIG. 37B). In contrast, non-cancer and PDAC cfDNA samples did not cluster separately when the bottom 50 gene set was used as features (FIG. 37D). Consistent with these findings, a prediction model built using top 50 gene set performed well with an AUC of 0.88 in classifying PDAC and non-cancer cfDNA samples correctly (FIG. 37C), unlike the model that used bottom 50 gene set as features, which had an AUC of 0.57. While the model trained with cfDNA 5hmC profiles performed best with an AUC of 0.919, inspection of normalized 5hmC signal (RPKM) from 37 features selected in cfDNA model demonstrates that PDAC cfDNA signal is overall admixed between non-cancer cfDNA and PDAC tissue (FIG. 38). Altogether, our results indicate that PDAC tumor tissue-derived features are useful in classification of cfDNA, while additional signals originating elsewhere can also provide additional classification capability.

C. Conclusions:

The work described in this Example was focused on the discovery of cfDNA specific hydroxymethylation-based biomarkers for use in the development of molecular diagnostic tests to detect pancreatic cancer at earlier stages. The data highlight the ability to detect differentially hydroxymethylated genes whose underlying biology shows association with both pancreas and cancer development as well as established trends in chromatin mark maps and other functional regions of the genome. Furthermore, regularized regression was used to build a predictive model from a comprehensive gene set that is highly variable, yielding an AUC of 0.919 along with two independent external data set validations with AUC of 0.921 and 0.943.

The 5hmC signal was readily found to overlap in gene-centric functional regions (enrichment in promoter, exons, UTR and TTS), as well as transposable elements like SINEs (enriched) and LINEs (depleted) (FIG. 23). Globally, PDAC cfDNA cohort had decreased number of peaks compared to non-cancer cfDNA cohort (FIG. 24), consistent with previous reports of decreased 5hmC in several types of cancer, including pancreatic cancer,

Indeed, decreased 5hmC was recently linked to malignant transformation in KRAS mutant pancreatic cells upon deactivation of p53, which are commonly observed in PDAC patients; see Lee and Pausova (2013), “Cigarette smoking and DNA methylation,” Front Genet. 4:132. Hydroxymethylcytosine changes in functional regions have also been reported in cfDNA from colorectal, esophageal, and lung cancers (see Zhang et al. (2018) Genomics, Proteomics & Bioinformatics 16: 187-199; Li et al. (2017), supra; and Boël et al. (1995) Immunity 2: 167-175). Consistent with these reports, we observed a decreased number of peaks in PDAC cfDNA relative to non-cancer cfDNA. Furthermore, we found PDAC-specific gains or losses in hydroxymethylation in functional regions in our data, including PDAC-specific 5hmC increase in 3′UTR, TTS and exons and 5hmC decrease in promoters detectable in cfDNA (FIG. 25). These changes were also observed in various pancreatic cancer stages (FIG. 39). In embryonic stem cells, 5-hydromethylation decreases in the promoter region have been shown to associate with elevated gene transcription (Nasir et al. (2011) Pancreas 40:627-33). An increase in disease relevant transcription is implicitly supported in our PDAC data by the 5hmC increase in gene-centric features mentioned earlier, as well as an apparent decrease of 5hmC in promoter regions (FIG. 25).

Dynamic changes in chromatin have been shown to control cell development and transition of cells with oncogenic potential; see Gieger et al. (2011) Nature 480: 201-08. Intersection of our 5hmC data with various chromatin states determined by ChIPseq in PDAC primary tumor tissues revealed 5hmC localization over active chromatin regions, most significantly active TSS and active enhancer regions (FIG. 26). Consistent with 5hmC changes over promoters, 5hmC decrease in PDAC cfDNA over active TSS regions also suggests disease specific increases in gene transcription via chromatin modifications, given the permissive transcriptional state associated with H3K4me3 (Stamenkovic et al. (1990) Nature 345: 74-77). Furthermore, we observed 5hmC decrease in weak enhancer regions identified by H3K27ac and H3K4me1 (FIG. 26). While 5hmC patterning around known functional elements of the genome suggests a broader interplay between hydroxymethylation and the epigenetic control of transcriptional processes, these results also indicate that 5hmC in cfDNA can be utilized for non-invasive monitoring of epigenomic dysregulation in PDAC.

As indicated in Section A, we employed coarse resolution of hydroxymethylation at gene-based level in PDAC, and yet were able to find genes with changes in 5hmC signals that highlighted pathways implicated in pancreatic cancer (FIG. 29). Majority of PDAC cancers harbor activating mutations in KRAS (^{˜}90%) and inactivating mutations in TP53 (^{˜}70%) (Baumgart et al. (2014) Cancer Discov 4: 688-701). Gene set enrichment analysis for the genes with increased 5hmC representation in gene body revealed several gene sets that are upregulated when KRAS is up or when p53 is down (FIG. 29, left panel). Furthermore, among genes with increased 5hmC were targets of transcription factors NFAT and FOXA (HNF3) (Table 11), previously reported to be involved in promoting pancreatic cancer initiation (Morris et al. (2019) Nature 573: 595-99) and metastasis (Yang et al. (2012) Oncogene 32: 663-69), respectively, via enhancer reprogramming. Investigation of genes with decreased 5hmC in PDAC cfDNA as compared to non-cancer cfDNA indicated enrichment of genes downregulated when KRAS was up. Genes related to whole blood and immune response were enriched among the genes with decreased 5hmC in PDAC cfDNA (FIG. 29, right panels). This would be consistent with increase in (tumor) tissue derived DNA in cfDNA in PDAC patients, diluting immune and blood cell derived DNA that makes up the majority of cfDNA in non-cancer individuals (see Tian et al. (2018) Cell Research 28(5): 597-600). These results altogether suggest that PDAC tissue-derived signals can be detected in cfDNA from cancer patients using 5hmC.

As found in Example 1, inspection of individual genes that were either significantly increased or decreased in 5hmC density revealed genes implicated in normal pancreas development, for instance the transcription factors GATA4, GATA6, PROX1, ONECUT1/2, in addition to genes whose increased expression is implicated in cancer, such as YAP1, TEAD, PROX1, ONECUT2, ONECUT1 and IGF1. The relative 5hmC increase in transcription factor genes like GATA4, GATA6, PROX1, ONECUT1/2, MEIS2, which were previously reported to be involved in early pancreatic development (see Huang et al. (2015) Nature Medicine 21: 1364-71; Burke et al. (2002) Mechanisms of Development 118: 147-55; Zhang et al. (2009) Mechanisms of Development 126: 958-73), suggest a reversion to a stem-like state in PDAC samples.

Genes with the most significant 5hmC increase in PDAC cfDNA were enriched in annotated relevant biology, and can therefore be used to build regularized regression models with a high performance (training AUC of 0.919 with external data set validation AUCs of 0.921 and 0.943). This provides confidence that the models used were measuring underlying biological signals relevant to PDAC. One such signal is the cancer antigen BAGE that is selected among the 37 features in our model.

1. A method for determining the likelihood that a patient has or will develop pancreatic ductal adenocarcinoma (PDAC) or will in a patient, comprising:
(a) obtaining a cell-free (cf)DNA sample from the patient;
(b) sequencing the patient cfDNA in a manner that identifies 5-hydroxymethylcytosine (5hmC) residues in the cfDNA;
(c) mapping the identified 5hmC residues to each of a plurality of loci in a reference hydroxymethylation profile, wherein each locus serves as a hydroxymethylation biomarker comprising a gene feature selected from a 3′UTR, a TTS, an intron, and a promoter, the gene feature associated with a gene implicated in pancreatic development, a gene related to cancer development, or a gene established to exhibit hyper-hydroxymethylation in PDAC;
(d) determining differences in extent and location between hydroxymethylation of the patient cfDNA and the reference hydroxymethylation profile at each locus; and
(e) using the extent and location of the differences, calculating a probability score representing the likelihood that the patient has or is at risk of developing PDAC.

2. The method of claim 1, the probability score is calculated using the extent and location of the differences in combination with at least one additional parameter correlated with a risk of developing PDAC.

3. The method of claim 2, wherein the at least one additional parameter is selected from lesion size; lesion location; presence or absence of pancreatic inflammation; jaundice; presence or absence of other symptoms; patient age; weight; gender; ethnicity; family history; genetic mutations; diabetes; physical activity; diet; pro-inflammatory cytokine levels; and smoking status of the patient.

4. The method of claim 1, wherein the reference hydroxymethylation profile is a data set comprising a composite of hydroxymethylation profiles for a population group of individuals who have at least one shared characteristic.

5. The method of claim 4, wherein the at least one shared characteristic comprises having been diagnosed with PDAC.

6. The method of claim 4, wherein the at least one shared characteristic comprises having been diagnosed with pancreatitis.

7. The method of claim 4, wherein the at least one shared characteristic comprises a family history of PDAC.

8. The method of claim 4, wherein the at least one shared characteristic comprises having been diagnosed with diabetes.

9. The method of claim 4, wherein the at least one shared characteristic comprises cigarette smoking status.

10. The method of claim 4, wherein the at least one shared characteristic comprises obesity.

11. The method of claim 4, wherein the at least one shared characteristic comprises an age range.

12. The method of claim 1, wherein the cell-free DNA sample is extracted from a blood sample.

13. The method of claim 1, wherein the cell-free DNA sample is extracted from pancreatic cyst fluid.

14. The method of claim 1, wherein at least one hydroxymethylation biomarker exhibits an increase in hydroxymethylation level in the patient relative to the reference hydroxymethylation profile.

15. The method of claim 1, wherein at least one hydroxymethylation biomarker exhibits a decrease in hydroxymethylation level in a patient relative to the reference hydroxymethylation profile.

16. The method of claim 1, wherein the hydroxymethylation biomarkers comprise loci that are associated with one or more of the following genes: ADARB2-AS1, ANKRD36B, ASAH2B, ATG4B, ATP8B1, BOLA1, C11orf88, C17orf97, C1orf170, C3orf36, C8orf74, CAMSAP2, CCDC54, CCDC59, CKAP2, CLK2P, CRTC1, CSRP2, CYB5D1, DNAJC27, DYNAP, FAM166A, FAM188B, FAM196A, FAM86JP, FAT4, FBXO5, FGF2, FUT2, GAS2L2, GAS6, GGACT, GLRX5, GPX1, GPX5, HBD, HLA-A, HTR1F, IL36G, KANSL1, KCNH6, KCTD15, KLHL38, KLK2, KRT6B, LAMC1, LGALS14, LGALS8-AS1, LIFR, LINC00266-1, LINC00310, LOC100130452, LOC100130557, LOC100130894, LOC100288778, LOC100505633, LOC100505648, LOC100505738, LOC100652909, LOC389033, LOC90784, LRRC37A2, MED11, MRPL23-AS1, NAT8L, NEUROD1, NEUROG2, NME5, NOMO3, NPRL2, NXN, ODF3L1, ODF3L2, OSCP1, PARD6G, PGAM1, PLA2G2E, PLSCR4, PPAP2A, PPP1R15A, PPP1R3E, RASL10B, REXO1L1, RIMBP3, RNF126P1, RNU6-76, RPP25, RPS27, SH3PXD2B, SHISA4, SLC25A38, SLC4A1, SLCO5A1, SPDEF, SRSF6, STRA6, SYNM, TBCB, TDRD6, TEX26, TMEM253, TNFSF13B, TTC14, TUBA4A, UBB, VAMP8, VGLL2, WASH2P, WNT9B, XBP1, and ZNF789.

17. The method of claim 1, wherein the hydroxymethylation biomarkers comprise loci that are associated with one or more of the following genes: GATA4, GATA6, PROX1, ONECUT2, YAP1, TEAD1, ONECUT2/ONECUT1-TCGA, IGF1, and IGF2.

18. The method of claim 1, wherein the hydroxymethylation biomarkers comprise loci that are associated with a possible mutation in a gene selected from BRCA2, BRCA1, CDKN2A, ATM, STK11, PRSS1, MLH1, PALB2, KRAS, CDKN2A, TP53, SMAD4, and combinations thereof

19. The method of claim 1, wherein step (c) comprises generating an initial patient hydroxymethylation profile.

20. The method of claim 19, wherein the patient has had an imaging scan in which a pancreatic lesion was identified.

21. The method of claim 20, wherein the reference hydroxymethylation profile represents a composite of hydroxymethylation profiles for a plurality of individuals who have had a pancreatic lesion identified in an imaging scan.

22. The method of claim 21, further including ascertaining a change in the pancreatic lesion by repeating steps (a) through (e) at a later time to generate a later patient hydroxymethylation profile and comparing the later hydroxymethylation profile with the initial patient hydroxymethylation profile to ascertain hydroxymethylation profile changes.

23. The method of claim 22, further including making a determination whether to implement a change in treatment of the patient based on the hydroxymethylation profile changes.

24. The method of claim 23, wherein the change in treatment comprises beginning treatment, modifying treatment, or ending treatment.

25. The method of claim 1, wherein the probability score is calculated using a logistic regression analysis of the differences in hydroxymethylation level at each of the hydroxymethylation biomarkers.

26. A method for reducing the risk that a pancreatic lesion surgically removed from a patient is benign, comprising, prior to surgery,
(a) obtaining a cell-free (cf)DNA sample from the patient;
(b) sequencing the patient cfDNA in a manner that identifies 5-hydroxymethylcytosine (5hmC) residues in the cfDNA;
(c) mapping the identified 5hmC residues to each of a plurality of loci in a reference hydroxymethylation profile, wherein each locus serves as a hydroxymethylation biomarker comprising a gene feature selected from a 3′UTR, a TTS, an intron, and a promoter, the gene feature associated with a gene implicated in pancreatic development, a gene related to cancer development, or a gene established to exhibit hyper-hydroxymethylation in PDAC;
(d) determining differences in extent and location between hydroxymethylation of the patient cfDNA and the reference hydroxymethylation profile at each locus; and
(e) using the extent and location of the differences, calculating a probability score representing the likelihood that the pancreatic lesion is benign; and
(f) carrying out surgical resection of the pancreatic lesion only if the probability score is greater than a value corresponding to a low risk of cancer.

27. A kit for determining the presence of or likelihood of developing pancreatic ductal adenocarcinoma (PDAC) in a patient, comprising:
at least one reagent for the determination of hydroxymethylation level at each of a plurality of hydroxymethylation biomarker loci in a cell-free DNA sample;
a solid support for capturing affinity-tagged 5hmC-containing cell-free DNA in the sample; and
written instructions for the use of the at least one reagent and the solid support in carrying out the method of claim 1.

28. The method of claim 1, comprising determining the likelihood that an individual at risk for developing PDAC has PDAC.

29. The method of claim 28, further including, prior to step (a), identifying the patient as being at risk for developing PDAC from one or more parameters selected from: an identified pancreatic lesion; pancreatic inflammation; jaundice; age; weight; gender; ethnicity; family history; genetic mutations; diabetes; physical activity; diet; pro-inflammatory cytokine levels; and cigarette smoking.

30. The method of claim 29, further including determining the likelihood that the individual has at least one additional type of cancer.

31. The method of claim 30, wherein the at least one additional type of cancer is selected from: bladder cancer; cancers of the blood and bone marrow; brain cancer; breast cancer; cervical cancer; colorectal cancer; esophageal cancer; liver cancer; lung cancer; ovarian cancer; prostate cancer; renal cancer; skin cancer; testicular cancer; thyroid cancer; and uterine cancer.

32. In a multi-cancer test that determines the likelihood that an individual has PDAC and at least one additional type of cancer selected from colorectal, esophageal, lung, and liver cancer, the improvement which comprises determining the likelihood that the individual has PDAC by carrying out the method of claim 1.

33. The improved multi-cancer test of claim 32, further including eliminating false positives for the at least one additional type of cancer prior to step (a).

34. The improved multi-cancer test of claim 31, further including eliminating false negatives for the at least one additional type of cancer prior to step (a).
